(19) United States
(12) Patent Application Publication
(10) Pub. No.: US 2026/0130822 A1
(43) Pub. Date: May 14, 2026

(54) MANUAL CARDIOPULMONARY RESUSCITATION DUTY CYCLE GUIDANCE

(71) Applicant: Stryker Corporation, Portage, MI (US)

(72) Inventors: Olibhéar McAlister, Belfast (GB); Adam Harvey, Hillsborough (GB); Ben McCartney, Carrickfergus (GB); Kristian Solem, Trelleborg (SE)

(73) Assignee: Stryker Corporation, Portage, MI (US)

(21) Appl. No.: 19/435,148

(22) Filed: Dec. 29, 2025

Related U.S. Application Data
(63) Continuation-in-part of application No. 19/251,598, filed on Jun. 26, 2025.
(60) Provisional application No. 63/665,148, filed on Jun. 27, 2024.
(60) Provisional application No. 63/746,882, filed on Jan. 17, 2025.

(51) Int. Cl. A61H 31/00; A61N 1/39

(52) U.S. Cl. CPC A61H 31/005; A61H 31/007; A61H 2201/10; A61H 2201/5007; A61H 2201/5043; A61H 2201/5048; A61N 1/3904

(57) ABSTRACT

CPR duty cycle guidance systems and associated techniques for guiding a rescuer during manual CPR are disclosed. An example CPR duty cycle guidance system includes an output device(s) and a processor(s). The processor(s) is configured to generate a signal(s) indicative of a duty cycle of chest compressions, wherein the duty cycle represents a ratio of a first time period of a compression phase of a compression-decompression cycle to a second time period of a decompression phase of the compression-decompression cycle, and cause the output device(s) to output the signal(s) as guidance for a rescuer who is manually administering the chest compressions to a subject.

### CROSS-REFERENCE TO RELATED APPLICATIONS

This application is a continuation-in-part of U.S. application Ser. No. 19/251,598, titled “DUTY CYCLE OPTIMIZATION IN CARDIOPULMONARY RESUSCITATION SYSTEMS” and filed on Jun. 26, 2025, which claims the benefit of U.S. Provisional Application No. 63/746,882, titled “DUTY CYCLE OPTIMIZATION IN CARDIOPULMONARY RESUSCITATION SYSTEMS” and filed on Jan. 17, 2025, and the benefit of U.S. Provisional Application No. 63/665,148, titled “Optimal CPR Duty Cycle of the LUCAS Device” and filed on Jun. 27, 2024. U.S. application Ser. Nos. 19/251,598, 63/746,882, and 63/665,148 are incorporated by reference herein in their entirety.

### BACKGROUND

During cardiac arrest, when a subject's heart stops beating or beats too ineffectively to circulate blood to the brain and other vital organs, cardiopulmonary resuscitation (CPR) can be an effective treatment that saves the life of the subject, if performed properly. CPR involves administering repeated chest compressions to the chest of the subject, which causes blood to circulate through the body of the subject. Historically, CPR has been performed manually by a rescuer placing their hands on the chest of the subject and repeatedly applying a downward force to the chest. However, the efficacy of manual CPR is dependent upon rescuers being trained to administer high quality CPR.

To help ensure that CPR is administered properly on a more consistent basis, and to alleviate some of the burden on rescuers at a rescue scene, various CPR systems have been developed. One example of an existing CPR system is a mechanical chest compression device (also known as a “mechanical CPR device”) that administers automated chest compressions to a subject. Two types of mechanical chest compression devices in use today include piston-based devices, which utilize a piston to compress the subject's chest, and vest-based devices, which utilize a load-distributing band to compress the subject's chest. Another example of a CPR system is a CPR feedback system, which may utilize a device placed on the subject's chest to monitor the chest compressions that a rescuer is manually administering to the subject, and to facilitate the provisioning of real-time feedback to the rescuer to adjust their technique, as needed.

### BRIEF DESCRIPTION OF THE DRAWINGS

FIG. 1 illustrates an environment including an example mechanical chest compression device that is configured to administer CPR treatment to a subject. FIG. 1 also illustrates a waveform exhibiting an example duty cycle of the mechanical chest compression device.

FIG. 2 illustrates an environment including an example CPR feedback system that is configured to provide feedback for a rescuer to dynamically adjust a duty cycle of chest compressions that the rescuer is manually administering to the subject.

FIG. 3 illustrates example hold periods of a waveform exhibiting an example duty cycle of chest compressions associated with CPR treatment.

FIG. 4 illustrates a waveform exhibiting an example duty cycle of a mechanical chest compression device, the waveform including a decompression phase that involves active decompression.

FIG. 5 illustrates a time of aortic valve closure exhibited in a blood pressure signal.

FIG. 6 illustrates a time of aortic valve closure exhibited in an ultrasound signal.

FIG. 7 illustrates a time of aortic valve closure exhibited in a photoplethysmography (PPG) signal.

FIG. 8 illustrates a time of aortic valve closure exhibited in an audio sound signal.

FIG. 9 illustrates an example process for controlling movement of a chest compressing mechanism of a mechanical chest compression device based on an evaluation of one or more criteria during CPR.

FIG. 10 illustrates an example process for implementing an iterative optimization algorithm to dynamically adjust a duty cycle of a mechanical chest compression device.

FIG. 11 illustrates an example process for providing, via a CPR feedback system, feedback for a rescuer to dynamically adjust a duty cycle of chest compressions that the rescuer is manually administering to the subject.

FIG. 12 illustrates an example process for implementing an iterative optimization algorithm to dynamically adjust one or more chest compression parameters associated with CPR treatment.

FIG. 13 illustrates an example external defibrillator configured to perform various functions described herein.

FIG. 14 illustrates an example mechanical chest compression device configured to perform various functions described herein.

FIG. 15 illustrates an environment including an example CPR duty cycle guidance system that is configured to provide duty cycle guidance to a rescuer who is manually administering chest compressions to the subject.

FIG. 16 illustrates an example visual signal presented on a display to provide duty cycle guidance to a rescuer who is manually administering chest compressions to the subject.

FIG. 17 illustrates an example visual signal presented on a display to provide duty cycle guidance to a rescuer who is manually administering chest compressions to the subject.

FIG. 18 illustrates an example visual signal presented on a display to provide duty cycle guidance to a rescuer who is manually administering chest compressions to the subject.

FIGS. 19A-19C illustrate example waveforms presented on a display to provide duty cycle guidance to a rescuer who is manually administering chest compressions to the subject.

FIG. 20 illustrates an example visual signal presented on a display to provide duty cycle guidance to a rescuer who is manually administering chest compressions to the subject.

FIGS. 21A and 21B illustrate an example visual signal presented on a display to provide duty cycle guidance to a rescuer who is manually administering chest compressions to the subject.

FIG. 22 illustrates an example signal(s) (audible and/or haptic) that may be output (via a speaker(s) and/or a haptic actuator(s)) to provide duty cycle guidance to a rescuer who is manually administering chest compressions to the subject.

FIG. 23 illustrates an example signal(s) (audible and/or haptic) that may be output (via a speaker(s) and/or a haptic actuator(s)) to provide duty cycle guidance to a rescuer who is manually administering chest compressions to the subject.

FIG. 24 illustrates an example process for providing, via a CPR duty cycle guidance system, guidance to a rescuer who is manually administering chest compressions to the subject.

FIG. 25 illustrates an example process for providing, via a CPR duty cycle guidance system, visual guidance to a rescuer who is manually administering chest compressions to the subject.

FIGS. 26A-26C illustrate an example process for providing, via a CPR duty cycle guidance system, guidance to a rescuer who is manually administering chest compressions to the subject.

FIG. 27 illustrates an example process for providing, via a CPR duty cycle guidance system, audible and/or haptic guidance to a rescuer who is manually administering chest compressions to the subject.

FIG. 28 illustrates the example mechanical chest compression device depicted in FIG. 1, FIG. 28 depicting various example components of the mechanical chest compression device to, among other things, record sounds (e.g., internal body sounds, environment sounds, etc.) and dynamically adjust a duty cycle of a mechanical chest compression device.

FIG. 29 illustrates the example CPR feedback device depicted in FIG. 2, FIG. 29 depicting various example components of the CPR feedback device to, among other things, record sounds (e.g., internal body sounds, environment sounds, etc.) and to output a signal(s) to provide duty cycle guidance to a rescuer who is manually administering chest compressions to the subject.

FIG. 30 illustrates audio signals generated during administration of CPR to a subject by a mechanical chest compression device, the audio signals including a first audio signal generated using an auscultation device(s) disposed on a thorax of the subject, and a second audio signal generated using a microphone(s) in an environment of the subject.

### DETAILED DESCRIPTION

Various implementations described herein relate to cardiopulmonary resuscitation (CPR) systems and/or CPR devices that are configured to operate in association with a particular duty cycle. As used herein, a “duty cycle” associated with a CPR system and/or a CPR device represents a ratio of a first time period of a compression phase of a compression-decompression cycle to a second time period of a decompression phase of the compression-decompression cycle. Existing mechanical chest compression devices utilize a 50:50 duty cycle, with compression and decompression phases of equal duration. That is, during one compression-decompression cycle, 50% of the cycle time is spent in the compression phase and 50% of the cycle time is spent in the decompression phase.

Existing CPR feedback systems also adhere to the same 50:50 duty cycle, which has historically been promoted in CPR guidelines notwithstanding a lack of sufficient scientific evidence backing recommendations to utilize the 50:50 duty cycle in CPR treatment. As of this writing, there is no consensus within the scientific CPR community for what the optimal duty cycle of CPR treatment should be, and most rescuers and CPR systems default to using a 50:50 duty cycle in the absence of recommendations (e.g., recommendations in CPR guidelines) to the contrary. However, administering CPR at a 50:50 duty cycle can worsen patient outcomes. One reason for this is because the chest compressions may be held too long (e.g., too long after the systolic phase of the heart has ended and/or too long after the aortic valve has closed, in cases where the CPR creates a pressure gradient that causes the aortic valve to close, or in cases where the recipient of the chest compressions has some spontaneous cardiac activity), which means that chest compressions administered at a 50:50 duty cycle are often preventing the heart from filling with blood during the diastolic phase of the heart.

Implementations of the present disclosure address these and other problems through CPR systems and/or CPR devices that are configured to operate in association with a particular duty cycle. In order to illustrate how to determine a duty cycle of chest compressions associated with CPR treatment that will improve patient outcomes, the next few paragraphs will describe the physiological aspects of a spontaneously beating heart, as well as the physiological aspects of CPR on a subject in cardiac arrest.

Physiological Aspects of a Spontaneously Beating Heart: A spontaneously beating heart has two different phases during a cardiac cycle, the systolic phase and the diastolic phase. The systolic phase is an energy consuming phase and is often referred to as “the muscle contraction phase.” The contraction of the heart muscle leads to the ejection of blood from the ventricles of the heart. The diastolic phase, on the other hand, is a muscle relaxation phase and is often referred to as “the filling phase.” The diastolic phase is about twice as long as the systolic phase. That is, to maximize circulation of blood, the heart is constructed and optimized to work with shorter time in systole compared to the time in diastole. In this disclosure, the relationship between the systolic phase and the diastolic phase is defined as the “cardiac duty cycle.” In other words, the cardiac duty cycle, as used herein, means the duty cycle of the spontaneously beating heart, which represents a ratio of a first time period of systole to a second time period of diastole for an individual cardiac cycle. A spontaneously beating heart may spend about 33% of the time in systole and about 67% of the time in diastole, which can be expressed as a 33:67 cardiac duty cycle.

A spontaneous cardiac cycle begins with the systolic phase, which starts with the closing of the mitral valve, followed by a short “isovolumetric contraction” period in which the volume in the ventricle is constant and the pressure increases due to ventricle contraction and all the heart valves being simultaneously closed. The pressure in the left ventricle increases to a value greater than the pressure in the aorta, which causes the aortic valve to open. Next, blood is ejected from the heart into the aorta. The ejection of blood is caused by a contraction of the ventricle. During the contraction, the ventricle is compressed and the volume inside the ventricle decreases. The decrease in volume is a measure of cardiac function and performance and is an estimate of a measure called “ejection fraction (EF).” A spontaneously beating heart may have an EF of about 60%, meaning that 60% of the blood volume inside the ventricle is ejected in one heartbeat, and the other 40% of the blood volume remains inside the ventricle until the next heartbeat (by comparison, a severe heart failure patient may have an EF as low as 20%). The decrease in volume inside the ventricle continues as the ventricle is further compressed. The compression of the ventricle also contributes to an increase in pressure in the ventricle. Once the ventricle is fully compressed and the volume inside the ventricle does not decrease any further, a small amount of blood continues to be ejected from the ventricle due to the momentum of the blood and a pressure gradient between the ventricle and the aorta. The pressure inside the ventricle starts to drop rapidly, leading to the closing of the aortic valve shortly after the ventricle is fully compressed. The aortic valve closes as soon as the pressure inside the ventricle drops below the pressure in the aorta. The systolic phase ends with the closing of the aortic valve, and, from that time point forward, no additional blood is ejected from the heart.

The diastolic phase begins with a short “isovolumetric relaxation” period in which the volume in the ventricle is constant and the pressure decreases due to the heart muscle relaxing and all the heart valves being simultaneously closed. The pressure in the left ventricle decreases to a value lower than the pressure in the atria, causing the mitral valve to open, which marks the beginning of a period when blood starts flowing into the ventricle from the atria. The filling of blood into the ventricle is caused by a relaxation of the ventricle. During the relaxation of the ventricle, the ventricle is decompressed and the volume inside the ventricle increases, leading to a filling of blood into the ventricle until the blood volume reaches the starting amount of blood volume for the next heartbeat, which marks the end of the spontaneous cardiac cycle.

Physiological Aspects of CPR on a Cardiac Arrest Patient: A cardiac arrest patient does not have the systolic and diastolic phases during a cardiac cycle of a spontaneously beating heart. Instead, the heart of a cardiac arrest patient may not contract properly, which leads to little-to-no blood being ejected from the heart into the aorta, resulting in a lack of adequate blood circulation. In order to restore blood circulation, CPR can be performed on the patient by compressing and decompressing the chest of the subject at a given rate and depth. The general belief is that the compressions on the chest will create a systolic phase in the heart, and that the decompressions will create a diastolic phase. Thus, one might expect the heart of a cardiac arrest patient to remain in the systolic phase throughout the entire duration of one chest compression, and that systole ends when the decompression of the chest begins. However, this is a misconception.

During CPR, while the chest of the patient is being compressed during a first portion of the compression phase of a compression-decompression cycle, the ventricle of the heart is compressed. During a second portion of the compression phase, shortly after a time at which a target compression depth is reached and the chest of the subject is held in the compressed state, the aortic valve of the heart closes. For example, at a compression rate of 100 compressions per minute, the aortic valve should close shortly after a target compression depth is reached, which may occur at about 0.15 seconds into compression-decompression cycle. In other words, the expected time for the closing of the aortic valve is around 0.2 seconds into the compression-decompression cycle, assuming a compression rate of 100 compressions per minute is utilized. After the aortic valve closes, no more blood is ejected from the heart, and if the chest of the subject is held in the compressed state for the remainder of the compression phase, and if the compression is released halfway through the compression-decompression cycle (i.e., utilizing a 50:50 duty cycle at a compression rate of 100 compressions per minute), the time period from about 0.2 seconds to about 0.3 seconds into the compression-decompression cycle is a wasted time period when nothing productive is being done to help the subject recover from cardiac arrest because no blood is being ejected from the heart and because the heart is being prevented from filling with blood during this time period.

In the light of the foregoing, the present disclosure describes techniques for setting a duty cycle and/or adjusting the duty cycle in CPR systems and/or CPR devices. In one example, a duty cycle of a CPR system and/or a CPR device includes a compression time period that is shorter than a decompression time period, which reduces, if not eliminates, a “wasted time period” during which the heart of the CPR recipient is neither ejecting blood nor being filled with blood. In some examples, a duty cycle of a CPR system and/or a CPR device is within a range of about 35:65 to about 45:55. In some examples, a duty cycle of a CPR system and/or a CPR device is about 40:60. A non-50:50 duty cycle with a compression phase that is shorter than a decompression phase not only reduces, if not eliminates, the wasted time period at the end of the compression phase during which the heart is neither ejecting blood nor being filled with blood, but also creates extra time during the relatively longer decompression phase during which the heart is able to fill with blood. Therefore, in some implementations, a duty cycle implemented in accordance with the present disclosure increases cardiac filling, and, as a consequence, increases cardiac output and improves blood pressure, circulation, and perfusion in the subject who is receiving CPR treatment.

In some examples, a duty cycle of a CPR system and/or a CPR device includes a compression phase that ends within a threshold amount of time after closure of the aortic valve (e.g., within a threshold time after about 0.2 seconds into a compression-decompression cycle, assuming a compression rate of 100 compressions per minute is being utilized). However, there is a limit to how short the compression phase can be. For example, if the compression phase ends before the closing of the aortic valve, it is possible that blood will be drawn back into the heart from the aorta, thereby counteracting the ejection of blood into the aorta. Thus, optimization of the duty cycle of a CPR system and/or a CPR device, as described herein, may depend on one or more criteria being satisfied, such as the aortic valve of the heart closing, and/or an amount of time since a start of the compression phase expiring (where the amount of time relates to the closing of the aortic valve). In some examples, closure of the aortic valve of the heart is detected in real-time during an individual compression-decompression cycle. In some examples, the time at which the aortic valve of the heart closes during CPR is estimated during CPR, such as by analyzing one or more parameters associated with the subject and sensed over a duration of multiple compression-decompression cycles. In some examples, the CPR system and/or the CPR device is preprogramed to utilize or otherwise target a fixed duty cycle, which may be determined from an estimate of a time at which the aortic valve is expected to close, based on previously collected clinical data and/or data simulations or models. Although closure of the aortic valve is one example of a criterion that is to be satisfied to determine the duty cycle of a CPR system and/or a CPR device, it is to be appreciated that there are other examples of criteria that may be evaluated to determine whether and when to transition from the compression phase to the decompression phase for a given compression-decompression cycle during CPR treatment, and such examples of other criteria are described in more detail below.

An example mechanical chest compression device configured to implement the techniques described herein includes a processor(s) and a chest compressing mechanism configured to be disposed on a chest of a subject and to move for administering chest compressions to the subject. The processor(s) of the mechanical chest compression device is configured to cause the chest compressing mechanism to move for administering the chest compressions to the subject over a series of compression-decompression cycles, wherein a compression-decompression cycle of the series of compression-decompression cycles includes a compression phase that is shorter than a decompression phase. The processor(s) of the mechanical chest compression device is further configured to determine, during the compression phase, that a criterion is satisfied, and to cause the chest compressing mechanism to transition to movement that corresponds to the decompression phase in response to determining that the criterion is satisfied.

An example CPR feedback system configured to implement the techniques described herein includes a processor(s) and a sensor(s) configured to sense a parameter(s) associated with a subject as a rescuer is manually administering chest compressions to the subject. The processor(s) of the CPR feedback system is configured to determine, by analyzing the parameter(s) sensed by the sensor(s), that a criterion is satisfied, and, in response to determining that the criterion is satisfied, cause a signal(s) to be output as feedback for the rescuer to dynamically adjust a duty cycle of the chest compressions, wherein the duty cycle represents a ratio of a first time period of a compression phase of a compression-decompression cycle to a second time period of a decompression phase of the compression-decompression cycle.

In various implementations, a CPR system and/or a CPR device that is configured to operate in association with a duty cycle with a compression phase that is shorter than a decompression phase will not cause any negative influence on the ability to compress the chest of the subject and to eject blood from the heart. In fact, the ejection of blood from the heart will be more efficient using various techniques described herein, and the relatively shorter compression phase will cause an increase in cardiac output. Improvements in hemodynamic outcome and circulation are also realized by an increased cardiac filling time, which is at least partly due to the relatively longer decompression time period implemented using the techniques described herein, which results in improved venous return to the right side of the heart. Another benefit of a relatively shorter compression phase is that power consumption of a mechanical chest compression device is reduced (due to less time spent in the compression phase during which the mechanical chest compression device consumes the most power), leading to longer battery life and a prolonged operational lifetime of the mechanical chest compression device. Other clinical benefits include fewer injuries to subjects who receive CPR treatment, as well as an improved hemodynamic outcome in instances where the placement of the chest compressing mechanism (or the hand placement of the rescuer) is not optimized. In general, the systems, devices, and techniques described herein not only improve clinical outcomes in cardiac arrest patients, but also make CPR treatment safer for the subject and devices that administer automated chest compressions more energy efficient.

The present disclosure also describes a CPR duty cycle guidance system and techniques for providing duty cycle guidance to a rescuer who is manually administering chest compressions to a subject. An example CPR duty cycle guidance system configured to implement the techniques described herein includes an output device(s) and a processor(s). The processor(s) is configured to generate a signal(s) indicative of a duty cycle of chest compressions, wherein the duty cycle represents a ratio of a first time period of a compression phase of a compression-decompression cycle to a second time period of a decompression phase of the compression-decompression cycle. The processor(s) is further configured to cause the output device(s) to output the signal(s) as guidance for a rescuer who is manually administering the chest compressions to a subject. Outputting the signal(s) as guidance during CPR makes it easier for the rescuer to manually administer chest compressions at a target duty cycle (e.g., a non-50:50 duty cycle, such as a 40:60 duty cycle) for improved clinical outcomes in cardiac arrest patients, among the other benefits described herein.

Various implementations will now be described with reference to the accompanying figures.

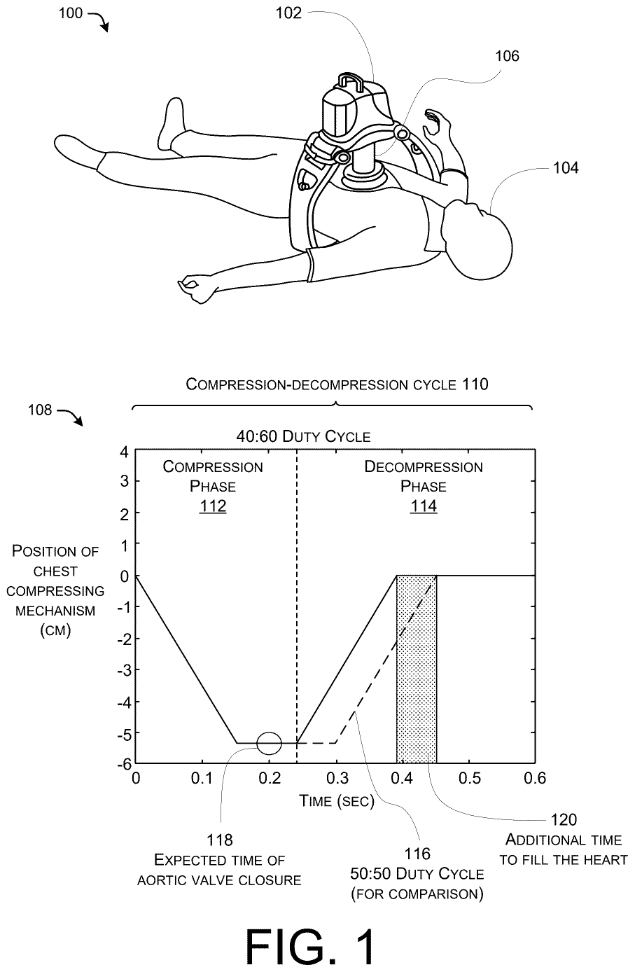

FIG. 1 illustrates an environment 100 including an example mechanical chest compression device 102 that is configured to administer CPR treatment to a subject 104. In various cases, the environment 100 is in an out-of-hospital environment. For example, the subject 104 may have experienced a medical emergency in a non-hospital, public space, such as a library, airport terminal, school, or office building. In some cases, the subject 104 has collapsed or otherwise lost consciousness within the environment 100. In some examples, the subject 104 has experienced one or more other types of symptoms associated with a serious health condition (e.g., cardiac arrest). As a result, a bystander may have contacted emergency services personnel in order to assist the subject 104 and to transport the subject 104 to a clinical environment, if necessary.

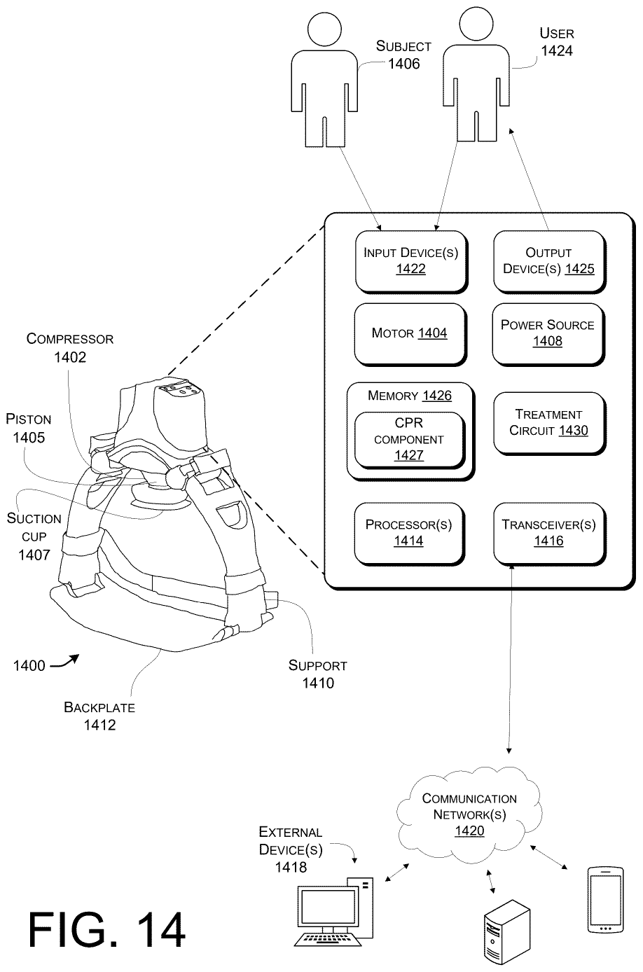

In various cases, a rescuer (not shown in FIG. 1) arrives at the rescue scene, secures the mechanical chest compression device 102 around the subject 104 (e.g., around a thorax thereof), and begins operating, and/or initiates autonomous operation of, the mechanical chest compression device 102. For example, the rescuer may provide input to the mechanical chest compression device 102 to set one or more chest compression parameters (e.g., compression rate, compression depth, etc.), and/or to start CPR treatment (e.g., autonomous chest compressions) via the mechanical chest compression device 102. Various example components of the mechanical chest compression device 102 shown in FIG. 1 are described in more detail below with respect to FIG. 14. In some examples, the mechanical chest compression device 102 includes a processor(s) (see FIG. 14) and a chest compressing mechanism 106. The chest compressing mechanism 106 is configured to be disposed on a chest of the subject 104 and to move for administering chest compressions to the subject 104. The example mechanical chest compression device 102 shown in FIG. 1 is a piston-based device. Accordingly, the chest compressing mechanism 106 illustrated in FIG. 1 is in the form of a piston, the piston being movable bidirectionally between various positions relative to the frame of the mechanical chest compression device 102 and/or relative to the subject 104 (e.g., the chest compressing mechanism 106 is configured to move up and down relative to the chest of the subject 104 when the subject 104 is lying on their back in a horizontal orientation). It is to be appreciated, however, that other types of mechanical chest compression devices may implement the techniques described herein, such as vest-based devices where the chest compressing mechanism includes a load-distributing band disposed around the thorax of the subject 104.

The processor(s) of the mechanical chest compression device 102 is configured to cause the chest compressing mechanism 106 to move for administering chest compressions to the subject 104 over a series of compression-decompression cycles. In some examples, the processor(s) is configured to execute computer-executable instructions stored in memory of the mechanical chest compression device 102 to control the movement of the chest compressing mechanism 106, as described herein. In some examples, the processor(s) is configured to control the movement of the chest compressing mechanism in accordance with one or more settings of the mechanical chest compression device 102, which may be adjusted by a user (e.g., a rescuer at the rescue scene). Additionally, or alternatively, such settings may be initialized automatically (without user intervention) by the mechanical chest compression device 102 at a time at which the mechanical chest compression device 102 is initialized (e.g., powered on) and/or adjusted automatically (without user intervention) on-the-fly during CPR treatment. The settings of the mechanical chest compression device 102 may relate to various chest compression parameters, such as compression rate, compression depth, compression force, compression velocity, compression acceleration, one or more hold periods during the compression and/or decompression phases, the duty cycle of the mechanical chest compression device 102, active decompression height, compression position on the chest, and/or the like.

FIG. 1 also illustrates a waveform 108 exhibiting an example duty cycle of the mechanical chest compression device 102. The waveform 108 is sometimes referred to herein as a “compression and decompression profile” because the waveform 108 plots the position of the chest compressing mechanism 106 (and/or a point or small area on the chest of the subject 104) over time during an individual compression-decompression cycle 110 of the mechanical chest compression device 102 as the device 102 is being used to administer chest compressions to the subject 104. In FIG. 1, for purposes of illustration, it is assumed that the mechanical chest compression device 102 is configured to administer chest compressions to the subject 104 at a rate of 100 compressions per minute, which means that the example duration of the compression-decompression cycle 110 is 0.6 seconds. However, it is to be appreciated that other compression rates may be implemented using the mechanical chest compression device 102 and/or the techniques described herein, which means that the duration of the compression-decompression cycle 110 may vary depending on the implementation. In some examples, the compression rate is fixed and the duty cycle of the mechanical chest compression device 102 is also fixed (e.g., the duty cycle does not change) throughout a CPR session. In some examples, the durations of individual compression-decompression cycles may vary dynamically throughout a CPR session and/or the duty cycle of the device 102 may vary dynamically throughout the CPR session.

As shown in the waveform 108 of FIG. 1, the compression-decompression cycle 110 includes a compression phase 112 followed by a decompression phase 114. Furthermore, when the mechanical chest compression device 102 uses the example 40:60 duty cycle exhibited in the waveform 108 of FIG. 1, the compression phase 112 is shorter than the decompression phase 114. For comparison, a dashed line corresponding to a waveform 116 exhibiting a 50:50 duty cycle is also shown in FIG. 1, where the first 0.3 seconds would represent the compression phase of the 50:50 duty cycle, and the last 0.3 seconds would represent the decompression phase of the 50:50 duty cycle. By contrast, the waveform 108, which exhibits the example 40:60 duty cycle, includes a compression phase 112 that is less than 0.3 seconds, and a decompression phase 114 that is greater than 0.3 seconds. That is, the compression phase 112 is shorter than the decompression phase 114.

The waveform 108 indicates that the chest compressing mechanism 106 starts at a first position of 0 centimeters (cm), and begins moving (e.g., downward, towards the subject 104) at a constant velocity until the chest compressing mechanism 106 arrives at a second position of about negative 5.3 cm. This second position (negative 5.3 cm) of the chest compressing mechanism 106 may represent a target compression depth, and the chest compressing mechanism 106 arrives at the target compression depth at about 0.15 seconds into the compression-decompression cycle 110. After arriving at the second position (negative 5.3 cm), the chest compressing mechanism 106 refrains from moving during a first hold period that spans from about 0.15 seconds to 0.24 seconds into the compression-decompression cycle 110. During this first hold period, the aortic valve of the heart of the subject 104 is expected to close at around 0.2 seconds into the compression-decompression cycle 110. This is shown in the waveform 108 of FIG. 1 as the expected time of aortic valve closure 118. Therefore, in the example of FIG. 1, the chest compressing mechanism 106 transitions to movement that corresponds to the decompression phase 114 within a threshold amount of time (e.g., about 0.04 seconds) after the expected time of aortic valve closure 118. Accordingly, at 0.24 seconds into the compression-decompression cycle 110, the chest compressing mechanism 106, which is at the second position (negative 5.3 cm), begins moving (e.g., upward, away from the subject 104) at a constant velocity until the chest compressing mechanism 106 returns to the first position of 0 cm at about 0.39 seconds into the compression-decompression cycle 110. After returning to the first position (0 cm), the chest compressing mechanism 106 refrains from moving during a second hold period that spans from about 0.39 seconds to 0.6 seconds, which marks the end of the compression-decompression cycle 110.

In some examples, the processor(s) of the mechanical chest compression device 102 is configured to determine, during the compression phase 112, that a criterion is satisfied, and to cause the chest compressing mechanism 106 to transition to movement that corresponds to the decompression phase 114 in response to determining that the criterion is satisfied. Various examples of the criterion that can be evaluated in this context are described in more detail below. In one example, determining that the criterion is satisfied includes determining, during the compression phase 112, and by analyzing a parameter(s) associated with the subject 104, that the aortic valve of the heart of the subject 104 has closed. The consensus within the scientific CPR community is that the aortic valve is open during the entire compression phase at a 50:50 duty cycle, and that the aortic valve closes when the compression phase ends. However, this is a misconception. In fact, the aortic valve will close earlier than the end of the compression phase at the 50:50 duty cycle. Accordingly, the time point at which the aortic valve will close can influence the determination of the duty cycle that will result in improved patient outcomes.

From a physiological standpoint, it makes little sense to continue the compression phase 112 long after the closing of the aortic valve, seeing as how no more blood is ejected from the left ventricle after the aortic valve closes, and, therefore, holding the compression too long will not improve cardiac output and hemodynamic performance. Accordingly, in some examples, the duty cycle of the mechanical chest compression device 102 can be selected such that the chest compressing mechanism 106 transitions to movement that corresponds to the decompression phase 114 immediate, or within a threshold amount of time (e.g., about 0.04 seconds), after the closing of the aortic valve, which increases, and in some cases maximizes, the time for filling the heart with blood during the decompression phase 114. That is, the selected duty cycle increases, and in some cases optimizes, the amount of time available for filling the heart with blood by increasing, and in some cases maximizing, the time period in the decompression phase 114 without negatively impacting the efficacy of the compression phase 112. The duty cycle of the mechanical chest compression device 102 that increases (e.g., maximizes) this time period in the decompression phase 114 will have little-to-no negative effects on the compression phase 112, and it will have positive effects on the decompression phase 114. In the example of FIG. 1, where a 40:60 duty cycle is used, the compression phase 112 is 20% shorter than the compression phase of a 50:50 duty cycle, but this shortening of the compression phase 112 will not influence the ability to compress the ventricle and eject blood from the heart during the compression phase 112 since the time period between 0.2 seconds and 0.3 seconds into the compression-decompression cycle 110 does not contribute to ejection of blood (assuming the aortic valve actually closes at about 0.2 seconds into the compression-decompression cycle 110). As a result, the decompression phase 114 is 20% longer than the decompression phase of a 50:50 duty cycle, which creates a longer time period for the heart to fill with blood. This lengthening of the decompression phase 114 is shown in FIG. 1 as the additional time to fill the heart 120.

The time at which the aortic valve closes is dependent upon multiple factors. Since, these factors influence the time point of the closing of the aortic valve, they also influence the optimal choice of the duty cycle of the mechanical chest compression device 102. Moreover, the closing of the aortic valve is dependent on the waveform 108, and, in particular, the shape of the waveform 108. For example, the closing of the aortic valve is dependent on the time at which the velocity (and, in some cases the acceleration) of the chest compressing mechanism 106 is zero (or close to zero) during the compression phase 112. In other words, the closing of the aortic valve is dependent on the time at which no further compression of the chest is made, even though the heart remains compressed as the chest is held in a compressed state. The time point for the closing of the aortic valve is also dependent on the duration of the compression phase 112 and the time and degree of acceleration and/or velocity of the chest compressing mechanism 106 (e.g., a value relating to the acceleration and/or velocity, such as the maximum value, the mean value, and/or the median value). In addition, the time point for the closing of the aortic valve may also be dependent on the compression rate of the mechanical chest compression device 102 (e.g., 100 compressions per minute, 110 compressions per minute, 120 compressions per minute, etc.) and/or the depth of the compressions (e.g., negative 5.0 cm, 5.5 cm, 6.0 cm, etc.). Accordingly, in some examples, the optimal choice of the duty cycle of the mechanical chest compression device 102 is dependent on the compression rate and/or the duration of the compression-decompression cycle 110. In examples where the optimal choice of the duty cycle is dependent on the compression rate and/or the duration of the compression-decompression cycle 110, the waveform 108 associated with the compression-decompression cycle 110 may be trapezoidal in shape, as depicted in FIG. 1. To illustrate, for the subject 104 depicted in FIG. 1, an optimal duty cycle may be a 40:60 duty cycle if chest compressions are being administered at a compression rate of 100 compressions per minute, whereas, for the same subject 104, an optimal duty cycle may be a 35:65 duty cycle (i.e., a different duty cycle) if chest compressions are being administered at a compression rate of 80 compressions per minute (i.e., a different compression rate), whereas, for the same subject 104, an optimal duty cycle may be a 45:55 duty cycle (i.e., a different duty cycle) if chest compressions are being administered at a compression rate of 120 compressions per minute (i.e., a different compression rate), and so on and so forth. In other words, in addition to being dependent on the subject 104, the optimal choice of the duty cycle may be dependent on the shape of the waveform 108 and/or on the compression rate and/or on the duration of the compression-decompression cycle 110.

Although the waveform 108 depicted in FIG. 1 is trapezoidal in shape, this is non-limiting, as the waveform 108 can be of any suitable shape, such as a sinusoidal waveform, a triangular-shaped (e.g., sawtooth) waveform, a rectangular-shaped waveform, a square-shaped waveform, or a waveform having any suitable polygonal shape. That being said, a duty cycle with a shorter compression period (e.g., a 40:60 duty cycle), as compared to a 50:50 duty cycle, improves patient outcomes if an approximately square-shaped waveform or a trapezoidal-shaped waveform (e.g., the waveform 108 shown in FIG. 1) is used, as opposed to other shapes, such as a sinusoidal-shaped waveform, for example. Again, this is because the closing of the aortic valve is dependent on the shape of the waveform 108 utilized.

It is to be appreciated that the time point for the closing of the pulmonary valve is associated with the time point for the closing of the aortic valve. Therefore, wherever closure of the “aortic valve” is discussed herein, the same holds true for the closing of the pulmonary valve, and vice versa. In other words, the criterion to be satisfied for transitioning from the compression phase 112 to the decompression phase 114 may include a determination, during the compression phase 112, that the pulmonary valve of the heart of the subject 104 has closed, as the closure of the aortic valve and the pulmonary valve occurs at similar times during CPR. Thus, all instances of “aortic valve” herein may be used interchangeably with “pulmonary valve.”

A duty cycle for the mechanical chest compression device 102 with a compression phase 112 that is shorter than the decompression phase 114 generates a better clinical hemodynamic outcome for the subject 104, seeing as how the duty cycle is better aligned with the physiological aspects of CPR on subject 104 in cardiac arrest. The relatively longer decompression phase 114 leads to improved cardiac output, blood pressure, circulation, and perfusion as a result of improved cardiac filling. Perfusion of the heart, also referred to as coronary perfusion, occurs during the diastolic phase in a normal beating heart. During the systolic phase in a normal beating heart, blood entering the coronary arteries (the blood vessels that supply blood to the heart) is inhibited e.g. by the stiffened and contracted heart muscle. So, blood is only supplied to the heart when the heart muscle is relaxed, i.e. the heart is only being perfused during the diastolic phase in a normal beating heart. Similar physiology applies to the heart during CPR, i.e. the heart is not being perfused during the compression phase 112 since the heart muscle and the coronary arteries are being compressed. So, blood is only supplied to the heart in the uncompressed state, i.e. the heart is only being perfused during the decompression phase 114 in CPR. The techniques described herein—which describe setting and/or adjusting a duty cycle in CPR systems and/or CPR devices to a duty cycle with a compression phase 112 that is shorter than the decompression phase 114—can enable improved and/or more effective perfusion of the heart. This is at least because a significantly longer (e.g., 20% longer) period of time is available in an uncompressed state (e.g., the decompression phase 114 is significantly longer, as compared to the decompression phase in conventional CPR systems/devices) allowing for coronary perfusion of the subject 104. The improved and/or more effective perfusion of the heart, obtained by the techniques described herein, occur in every single compression-decompression cycle 110 throughout the entire CPR duration. The improved and/or more effective perfusion may lead to increased likelihood of obtaining return of spontaneous circulation (ROSC) in the subject 104. It is to be appreciated that in reference to additional time to fill the heart 120, e.g. illustrated in FIG. 1 and FIG. 4, the additional time to fill the heart 120 is associated with additional time to perfuse the heart. Therefore, wherever “additional time to fill the heart” is discussed herein, the same holds true for “additional time to perfuse the heart”, and vice versa.

The compression phase 112 requires significantly more energy, and therefore the mechanical chest compression device 102 consumes more power, during the compression phase 112, as compared to the energy required, and the power consumed, during the decompression phase 114. This is because force is actively applied to the chest of the subject 104 during the entire compression phase 112, which is not the case during the decompression phase 114, and which requires driving the motor of the mechanical chest compression device 102 at a particular electrical current, thereby consuming more power than the power consumed to retract the chest compressing mechanism 106 during the decompression phase 114. The compression phase 112 duration is significantly reduced (as compared to a 50:50 duty cycle) using the techniques described herein. Therefore, the mechanical chest compression device 102, when using the techniques described herein, consumes less power, such as battery power, as compared to a mechanical chest compression device that uses a 50:50 duty cycle. This reduction in power consumption leads to a significant conservation of power resources (e.g., battery power). Thus, the battery life, as well as the operational lifetime of the mechanical chest compression device 102, is significantly increased using the techniques described herein (roughly a 20-25% increase in battery life/operational lifetime).

Patient injuries can occur due to delivery of energy and force to the chest of the patient over time, typically causing rib fractures and even sternum fractures. The techniques described herein, which relate to setting a duty cycle and/or adjusting the duty cycle in CPR systems and/or CPR devices to a duty cycle with a compression phase 112 that is shorter than the decompression phase 114, has the potential of reducing injuries (e.g., chest injuries), and thus causing less patient harm, since significantly less energy and force is delivered to the chest of the subject 104 over time.

Improper positioning of the mechanical chest compression device 102 may also lead to backflow through the aorta of the heart during the end of the compression phase 112, which significantly reduces the performance of the chest compressions. The techniques described herein may result in CPR quality that is less sensitive to improper positioning of the mechanical chest compression device 102, since the time for possible backflow (occurring in the end of the compression phase 112) is significantly reduced.

In some examples, ventilation may be provided during CPR. In these examples, ventilation can be administered in various modes. In one example, ventilation is administered during CPR in a 30:2 mode, which involves administering thirty chest compressions, then pausing the chest compressions and administering two breaths before resuming chest compressions. In another example, ventilation is administered during CPR in a continuous mode, which involves administering breaths at a particular ventilation rate (e.g., ten breaths per minute) without pausing the chest compressions. In the example of administering ventilation during CPR in a continuous mode, the breaths are delivered while chest compressions are being administered and without stopping the chest compressions, where one breath is delivered to the subject 104 during the decompression phase 114 following the administration of a chest compression. For instance, one breath may be delivered to the subject 104 after every tenth chest compression during CPR. The techniques described herein - which describe setting and/or adjusting a duty cycle in CPR systems and/or CPR devices to a duty cycle with a compression phase 112 that is shorter than the decompression phase 114 - can enable improved and/or more effective ventilation, at least in a continuous mode of administering ventilation during CPR. This is at least because a significantly longer (e.g., 20% longer) period of time is available (e.g., the decompression phase 114 is significantly longer, as compared to the decompression phase in conventional CPR systems/devices) for administering ventilation to the subject 104. Another consideration when performing ventilation in continuous mode is that the total administered ventilation volume may be split up into two or several smaller ventilation volumes during two or several consecutive compression-decompression cycles 110. The split in ventilation volume occurs when the period of time available for administering ventilation to the subject 104 (i.e., the decompression phase 114 in continuous mode) is too short in time. Thus, sufficient time to administer the total ventilation volume is not available in the decompression phase 114 resulting in the administration of the remaining undelivered ventilation volume that is delayed in time to the next decompression phase 114, following the compression phase 112. This delay in administration time will cause the total ventilation volume to be split up into two or several smaller ventilation volumes during two or several consecutive compression-decompression cycles 110. The ventilation effect will be reduced if a ventilation volume split occurs, even though the total ventilation volume in one ventilation cycle is the same, since split ventilation volume will have several smaller ineffective ventilation volumes instead of one larger effective ventilation volume. Multiple ventilation volume splits lead to that the smaller ventilation volumes between compressions will be similar to the “dead space volume” resulting in that no actual ventilation will occur since no air will reach the lungs, only the “dead space volume” will be replaced with the smaller administered ventilation volumes. Increasing the period of time available for administering ventilation to the subject 104 (i.e., the decompression phase 114), obtained by the techniques described herein, may result in a reduction in the number of splits in ventilation volume, i.e. the likelihood of having a ventilation volume split is reduced (i.e. ventilation is improved and/or more effective) with an increased period of time available for administering ventilation to the subject 104. The improved and/or more effective ventilation, at least in a continuous mode of administering ventilation during CPR, obtained by the techniques described herein, is not only the result of a significantly longer (e.g., 20% longer) period of time that is available (e.g., the decompression phase 114 is significantly longer, as compared to the decompression phase in conventional CPR systems/devices) for administering ventilation to the subject 104, but also the results of reducing the likelihood of having the ventilation volume split into several smaller ventilation volumes between compressions. It is to be appreciated that in reference to additional time to fill the heart 120, e.g. illustrated in FIG. 1 and FIG. 4, the additional time to fill the heart 120 is associated with additional time for administering ventilation. Therefore, wherever “additional time to fill the heart” is discussed herein, the same holds true for “additional time for administering ventilation”, and vice versa.

In some examples, a sound produced by the activation of the motor and/or the compression module of a conventional mechanical chest compression device operating at a 50:50 duty cycle is monotonic and periodic. This device-generated sound may be undesirable and/or unpleasant in certain settings. The techniques described herein - which describe setting and/or adjusting a duty cycle in CPR systems and/or CPR devices to a duty cycle with a compression phase 112 that is shorter than the decompression phase 114 - may result in the mechanical chest compression device 102 that generates a pleasant sound during operation, which can be perceived by humans as a natural sound. This sound may have a calming effect on people, such as bystanders and/or rescuers, in the vicinity of the mechanical chest compression device 102, thereby reducing anxiety and/or stress of the bystanders and/or rescuers.

In summary, the techniques described herein will improve clinical outcomes in cardiac arrest patients and provide a safer and more energy efficient treatment option, as compared to existing CPR systems that adhere to a fixed 50:50 duty cycle. Accordingly, the systems, devices, and techniques described herein effect an improvement on existing CPR system technology.

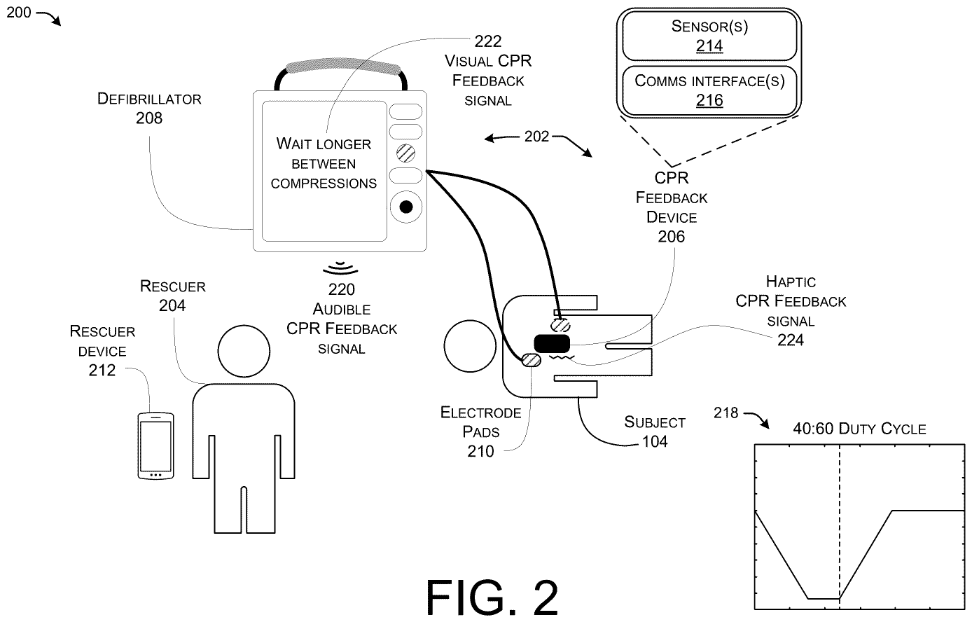

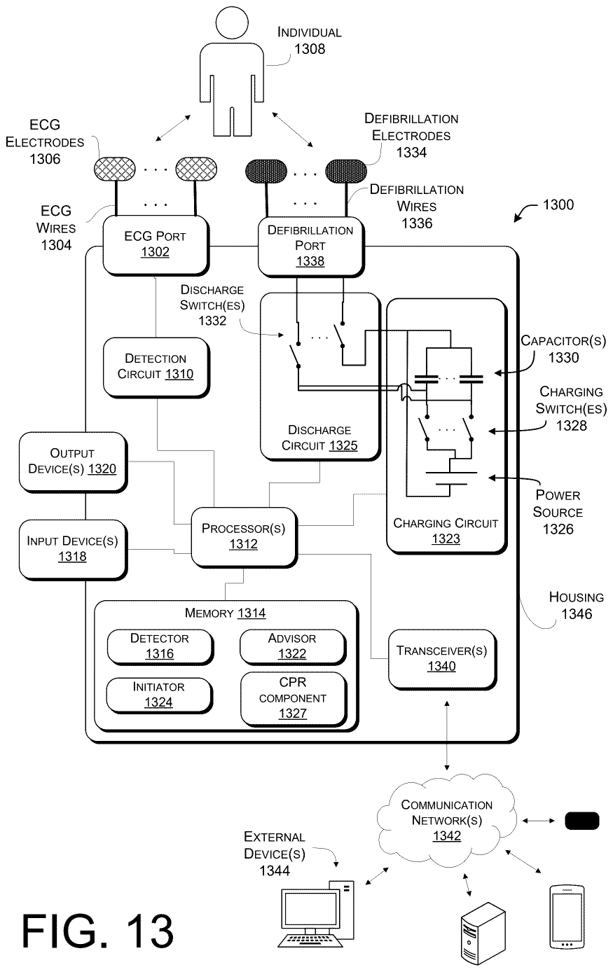

FIG. 2 illustrates an environment 200 including an example CPR feedback system 202 (sometime referred to herein as a “CPR duty cycle guidance system”) that is configured to provide duty cycle guidance for a rescuer 204 and/or feedback for a rescuer 204 to dynamically adjust a duty cycle of chest compressions that the rescuer 204 is manually administering to the subject 104. In some examples, the CPR feedback system 202 includes a CPR feedback device 206 and a defibrillator 208. Various example components of the defibrillator 208 shown in FIG. 2 are described in more detail below with respect to FIG. 13. In some examples, the rescuer 204 operates the defibrillator 208 to monitor and potentially treat the subject 104. In some examples, the defibrillator 208 is a monitor-defibrillator, an automated external defibrillator (AED), or other defibrillator device. In some examples, the defibrillator 208 is designed for operation by users without specialized medical knowledge. In some examples, the rescuer 204 has specific medical expertise. For instance, the rescuer 204 is an emergency medical services (EMS) professional, a physician, a nurse, or some other individual with specific medical training.

In some examples, the defibrillator 208 instructs the rescuer 204 to apply electrode pads 210 to the skin of the subject 104. For example, the electrode pads 210 may be adhered to skin on the chest of the subject 104. In some examples, the CPR feedback device 206 is integrated into the electrode pads 210. For example, the electrode pads 210 may be mechanically coupled to the CPR feedback device 206 and used as an all-in-one accessory of the defibrillator 208. In some examples, the CPR feedback device 206 is separate from the electrode pads 210 (e.g., a standalone CPR feedback device 206).

The defibrillator 208 includes one or more detection circuits (See FIG. 13), or measurement circuit(s), configured to detect one or more physiological parameters of the subject 104. The detection circuit(s), for instance, is configured to be connected to one or more sensors that are applied to the body of the subject 104. The sensor(s), for instance, is configured to generate electrical signals that are indicative of the physiological parameter(s) of the subject 104. The detection circuit(s) may infer the physiological parameter(s) based on the electrical signals generated by the sensor(s).

In some examples, the electrode pads 210 include electrodes that are configured to detect an electrical signal output by the heart of the subject 104 over time. A detection circuit in the defibrillator 208, for instance, is configured to detect an electrocardiogram (ECG) of the subject 104 based on the electrical signal detected by the electrode pads 210.

In some examples, the CPR feedback system 202 includes the CPR feedback device 206 and a rescuer device 212. In some examples, the rescuer device 212 is a mobile phone, a tablet, a laptop computer, a smart watch, a head-mounted display (HMD), or any other suitable user device operable by the rescuer 204. In some examples, the CPR feedback system 202 includes the CPR feedback device 206, the defibrillator 208, and the rescuer device 212.

As shown in FIG. 2, the CPR feedback device 206 may include one or more sensors 214 and one or more communication interfaces 216. The communication interface(s) 216 may include over one or more wired interfaces, one or more wireless interfaces, or a combination thereof. The communication interface(s) 216 configure the CPR feedback device 206 to communicate with (e.g., send signals, data, etc. to, and receive signals, data, etc. from) another device(s), such as the defibrillator 208 and/or the rescuer device 212. In some examples, the CPR feedback device 206 and the defibrillator 208 are configured to be wirelessly paired. Once paired, the CPR feedback device 206 is configured to communicate wirelessly with the defibrillator 208 over at least one wireless channel via the communication interface(s) 216. Additionally, or alternatively, the CPR feedback device 206 is configured to couple with the defibrillator 208 via one or more cables or wires, such as the cables that connect the electrode pads 210 to the defibrillator 208, and/or a separate cable(s) or wire(s). In this latter example, the CPR feedback device 206 is configured to communicate with the defibrillator 208 over the cable(s) or wire(s) via the communication interface(s) 216. In some examples, the CPR feedback device 206 and the rescuer device 212 are configured to be wirelessly paired or coupled via one or cables or wires in a similar fashion.

Various mechanisms can be utilized to pair the CPR feedback device 206 with the defibrillator 208 and/or the rescuer device 212. For example, automated pairing may involve the exchange of data and/or pairing requests/responses between the CPR feedback device 206 and the defibrillator 208 and/or between the CPR feedback device 206 and the rescuer device 212. As another example, the defibrillator 208 may receive an input signal from an operator (e.g., the rescuer 204) that selects the CPR feedback device 206 as a device to pair with the defibrillator 208, or vice versa. Similarly, the rescuer device 212 may allow an operator (e.g., the rescuer 204) to select the CPR feedback device 206 as a device to pair with the rescuer device 212, such as via an application (e.g., a CPR feedback application) executing on the rescuer device 212. In some cases, the defibrillator 208 and/or the rescuer device 212 detects an alternative signal (e.g., a flashing light pattern) from the CPR feedback device 206 that is indicated in a pairing request. In some cases, the defibrillator 208 and/or the rescuer device 212 is paired with the CPR feedback device 206 based, at least in part, on signaling to and/or from an intermediary device (not illustrated). In a particular example, two or more devices can be brought into proximity to (e.g., into contact with) each other in order to facilitating pairing (e.g., the defibrillator 208 with the CPR feedback device 206). For example, a “tap-to-pair” functionality may allow a user to bring the CPR feedback device 206 (or a component thereof) into close proximity to (e.g., into contact with) the defibrillator 208 (or a component thereof), or vice versa, and a short-range wireless protocol, such as BLUETOOTH, near-field communication (NFC), or the like, may be used to detect that the defibrillator 208 and CPR feedback device 206 are within a threshold distance of each other, and, in response, the defibrillator 208 and the CPR feedback device 206 may be paired. In some cases, an intermediary device (e.g., the rescuer device 212) may be brought into close proximity to (e.g., into contact with) the defibrillator 208 and/or the CPR feedback device 206 (e.g., by touching the intermediary device to both the defibrillator 208 and the CPR feedback device 206 sequentially), and a short-range wireless protocol may be used to detect these proximity events involving the intermediary device, and, in response, the defibrillator 208 and the CPR feedback device 206 may be paired.

The sensor(s) 214 of the CPR feedback device 206 is configured to sense one or more parameters associated with the subject 104 and/or with the CPR treatment (e.g., chest compressions) that the rescuer 204 is manually administering to the subject 104. For example, the sensor(s) 214 may include one or more accelerometers, inertial measurement unit(s) (IMU(s)), gyroscopes, compasses, pressure sensor(s) (e.g., force sensing resistor(s) (FSR(s))), proximity sensor(s) (e.g., capacitive sensor(s)), auscultation device(s)/sensor(s), microphone(s), and/or other sensors that are configured to sense one or more parameter(s) associated with the subject 104 as the subject 104 is receiving CPR treatment. The parameter(s) sensed by the sensor(s) 214 can include a compression parameter(s) (e.g., compression rate, compression depth, compression force, compression velocity, compression acceleration, hand position on the chest, etc.), an audible heart sound parameter(s) (e.g., the opening and/or closing of valves of the heart of the subject 104, such as the aortic valve, the pulmonary valve, the mitral valve, etc.).

In some examples, sensor(s) are connected to the defibrillator 208 to detect one or more physiological parameters of the subject 104. In some examples, such physiological parameter(s) may include a blood pressure parameter(s), a blood flow parameter(s), a blood oxygenation parameter(s), an end-tidal carbon dioxide (EtCO₂) parameter(s), an ultrasound parameter(s), a photoplethysmography (PPG) parameter, an audible heart sound parameter(s), an impedance parameter(s), and/or other physiological parameters associated with the subject 104. As described in more detail below, the defibrillator 208 and/or the rescuer device 212 may be configured to analyze one or more of these physiological parameters and/or one or more of the parameter(s) sensed by the sensor(s) 214 of the CPR feedback device 206 to determine whether a criterion is satisfied, and, if so, cause a signal(s) to be output as feedback for the rescuer 204 to dynamically adjust a duty cycle of the chest compressions that the rescuer 204 is manually administering to the subject 104. FIG. 2 illustrates an example waveform 218 exhibiting a target duty cycle (e.g., a 40:60 duty cycle), and the CPR feedback system 202 of FIG. 2 may be configured to evaluate one or more criteria (e.g., based on analyzing a parameter(s) sensed by a sensor(s)) to determine whether to provide feedback for the rescuer 204 to adjust the current duty cycle towards the target duty cycle, and when to provide such feedback to the rescuer 204.

In some examples, the defibrillator 208 is configured to output a signal(s) as guidance for the rescuer 204 and/or as the feedback for the rescuer 204 to dynamically adjust a duty cycle of the chest compressions. In some examples, the rescuer device 212 is configured to output a signal(s) as guidance for the rescuer 204 and/or as the feedback for the rescuer 204 to dynamically adjust a duty cycle of the chest compressions. The signal(s) that is output as guidance for the rescuer 204 and/or as the feedback for the rescuer 204 may include an audible signal(s), a visual signal(s), and/or a haptic signal(s). FIG. 2 illustrates an example audible signal(s) 220 that may be output by the defibrillator 208 as guidance for the rescuer 204 and/or as feedback for the rescuer 204 to dynamically adjust a duty cycle of the chest compressions. The audible signal(s) 220 may be output via a speaker(s) of the defibrillator 208. In some examples, the rescuer device 212 is configured to output a similar audible signal(s) 220 via a speaker(s) of the rescuer device 212. In some examples, the audible signal(s) 220 includes synthesized speech that provides verbal feedback (e.g., “wait longer between compressions,” “down, up, down, up, . . . ,” etc.), a rhythmic, repeating tone or beat (e.g., a metronome) and/or music with which the rescuer 204 can synchronize the chest compressions, and/or other types of audible signals. In some examples, a processor(s) of the CPR feedback system 202 (e.g., a processor(s) of the defibrillator 208 (See FIG. 13)) is configured to select a pattern from a set of predefined patterns, and the audible signal(s) 220 that is output is based on the selected pattern.

FIG. 2 further illustrates an example visual signal(s) 222 that may be output by the defibrillator 208 as guidance for the rescuer 204 and/or as feedback for the rescuer 204 to dynamically adjust a duty cycle of the chest compressions. The visual signal(s) 222 may be output via a display(s) of the defibrillator 208, one or more light emitting elements (e.g., a light emitting diode(s) (LED(s)) of the defibrillator 208, and/or other output devices of the defibrillator 208. In some examples, the rescuer device 212 is configured to output a similar visual signal(s) 222 via a display(s) of the rescuer device 212. In some examples, the visual signal(s) 222 includes text (e.g., “wait longer between compressions,” “down, up, down, up, . . . ,” etc.), a rhythmic, repeating visual indicia and/or flashing light with which the rescuer 204 can synchronize the chest compressions, and/or other types of visual signals. In some examples, a processor(s) of the CPR feedback system 202 (e.g., a processor(s) of the defibrillator 208) is configured to select a pattern from a set of predefined patterns, and the visual signal(s) 222 that is output is based on the selected pattern.

FIG. 2 further illustrates an example haptic signal(s) 224 that may be output by the CPR feedback device 206 as guidance for the rescuer 204 and/or as feedback for the rescuer 204 to dynamically adjust a duty cycle of the chest compressions. The haptic signal(s) 224 may be output via a haptic actuator(s) (e.g., a linear resonant actuator (LRA), an eccentric rotating mass (ERM), a piezoelectric actuator, etc.) of the CPR feedback device 206. In some examples, the defibrillator 208 and/or the rescuer device 212 is configured to cause the haptic signal(s) 224 to be output by the CPR feedback device 206, such as by transmitting an instruction(s) to output the haptic signal(s) 224. In some examples, the CPR feedback device 206 may include output devices (e.g., lights, speakers, etc.) to output the audible signal(s) 220 and/or the visual signal(s) 222 (described above) based on a similar instruction(s) received from the defibrillator 208 and/or the rescuer device 212. In some examples, the rescuer device 212 is configured to output the haptic signal(s) 224, such as a rescuer device 212 that is worn by the rescuer 204 (e.g., a smart watch with a haptic actuator). In some examples, the haptic signal(s) 224 includes a rhythmic, repeating vibration with which the rescuer 204 can synchronize the chest compressions, and/or other types of haptic signals. In some examples, a processor(s) of the CPR feedback system 202 (e.g., a processor(s) of the defibrillator 208) is configured to select a pattern from a set of predefined patterns, and the haptic signal(s) 224 that is output is based on the selected pattern.

Upon receiving the guidance and/or the feedback from the CPR feedback system 202, the rescuer 204 can utilize the guidance and/or the feedback to adjust their technique for administering the chest compressions to the subject 104. For example, by following a pattern that is being output audibly, visually, and/or haptically by the CPR feedback system 202 of FIG. 2, the rescuer 204 can adjust their technique by, for example, compressing the chest of the subject 104 to a target compression depth, pausing at the target compression depth, and releasing the compression for a longer period of time than the period of time that the rescuer compresses and holds the chest in the compressed state. In other words, the guidance and/or the feedback provided to the rescuer 204 may be specifically designed for the rescuer 204 to dynamically adjust a duty cycle of the chest compressions in accordance with the target duty cycle, regardless of whether or not the rescuer 204 is aware that the guidance and/or the feedback is specifically indicative of a target duty cycle and/or designed to adjust the duty cycle. That is, the rescuer 204 may not be aware that the guidance and/or the feedback is specifically designed for the rescuer 204 to dynamically adjust the duty cycle of the chest compressions, but the rescuer 204 is generally aware that the guidance and/or the feedback is designed to improve the quality of CPR they are administered to the subject 104.

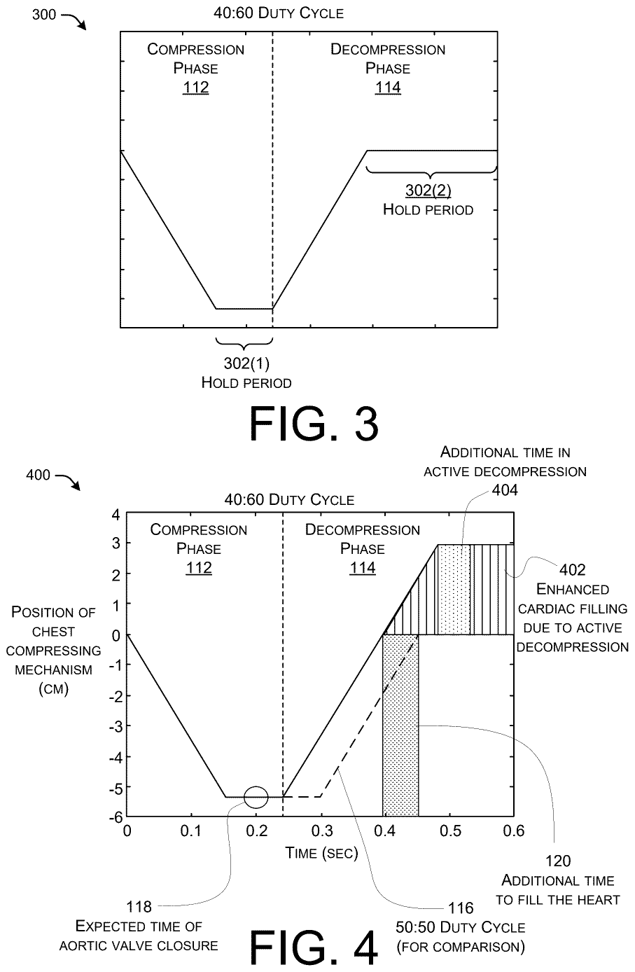

As mentioned above, the closing of the aortic valve during CPR is dependent on the waveform (e.g., the waveform 108), and, in particular, the shape of the waveform. For example, the closing of the aortic valve is dependent on the time at which the velocity (and, in some cases, the acceleration) of the chest compressing mechanism 106 (or the hands of the rescuer 204) is zero (or close to zero) during the compression phase. In other words, the closing of the aortic valve is dependent on the time at which no further compression of the chest is made, even though the heart remains compressed as the chest is held in a compressed state. FIG. 3 illustrates example hold periods of a waveform 300 exhibiting an example duty cycle of chest compressions associated with CPR treatment that is being administered by, or in association with the use of, a CPR system. For example, the waveform 300 includes two hold periods 302, a first hold period 302(1) at the end of the compression phase 112, and a second hold period 302(2) at the end of the decompression phase 114. Although two hold periods 302 are shown in the example waveform 300 of FIG. 3, it is to be appreciated that a waveform corresponding to a compression-decompression cycle may include more than two hold periods, as few as one hold period, or may omit hold periods altogether. That said, the shape of the waveform 300 shown in FIG. 3 is beneficial in combination with a duty cycle having a compression phase 112 that is shorter than the decompression phase 114, such as the example 40:60 duty cycle exhibited in the waveform 300. One reason for this is that the first hold period 302(1) (e.g., plateau period) in the waveform 300 allows the aortic valve to close before the compression phase 112 ends. In addition, the second hold period 302(2) (e.g., another plateau period) in the waveform 300 is preceded by a fast, complete recoil of the chest, and then, during the second hold period 302(2), the heart is allowed to fill with blood in a natural and undisturbed manner. When the chest compressing mechanism 106 of the mechanical chest compression device 102 moves in accordance with the waveform 300, the chest compressing mechanism 106 refrains from moving during the hold periods 302. When there is a hold period 302(1) at the end of the compression phase 112, the chest is not compressed any further during the hold period 302(1). When there is a hold period 302(2) at the end of the decompression phase 114, the chest is not decompressed any further during the hold period 302(2). In examples where a CPR feedback system 202 provides feedback to the rescuer 204 to dynamically adjust a duty cycle of the chest compressions towards a target duty cycle (e.g., the duty cycle exhibited in the waveform 300), the feedback for the rescuer 204 may include feedback to pause at an end of the compression phase 112 before transitioning to movement that corresponds to the decompression phase 114 and/or to pause at the end of the decompression phase 114 before transitioning to movement that corresponds to the compression phase 112 of a next (upcoming) compression-decompression cycle.

In some examples, a portion of the decompression phase 114 is associated with active decompression. Active decompression involves actively decompressing the chest of the subject 104 by pulling upward on the chest, or otherwise expanding the chest. FIG. 4 illustrates a waveform 400 exhibiting an example duty cycle of a mechanical chest compression device 102, the waveform 400 including a decompression phase 114 that involves active decompression. In some examples, the chest compressing mechanism 106 of the mechanical chest compression device 102 is a piston with a suction cup (See FIG. 14) disposed on a distal end of the piston, which is configured to pull upward on the chest of the subject 104. In FIG. 4, from the start of the compression-decompression cycle to about 0.39 seconds into the compression-decompression cycle, the waveform 400 is similar to the waveform 108 of FIG. 1. However, instead of the chest compressing mechanism 106 stopping at the first position of 0 cm and holding through the remainder of the compression-decompression cycle, the chest compressing mechanism 106 continues moving (e.g., upward, away from the subject 104) at the constant velocity past the first position (0 cm) until the chest compressing mechanism 106 arrives at a third position of about 3 cm. The chest compressing mechanism 106 arrives at the third position (about 3 cm) at about 0.47 seconds into the compression-decompression cycle. Accordingly, from about 0.39 seconds to about 0.47 seconds into the compression-decompression cycle, the chest compressing mechanism 106 is actively decompressing the chest of the subject 104. At 0.47 seconds into the compression-decompression cycle, the chest compressing mechanism 106 refrains from moving during a second hold period that spans from about 0.47 seconds to 0.6 seconds, which marks the end of the compression-decompression cycle.

The described duty cycle with a compression phase 112 that is shorter than the decompression phase 114, when combined with the implementation of active decompression, is particularly beneficial because the combination produces a synergistic positive effect on patient outcomes. Active decompression generates negative intrathoracic pressure between chest compressions, resulting in the active transport of blood into the thoracic cavity, the enhancement of venous return, and an increase in blood volume to the right side of the heart. This is shown in FIG. 4 as an enhanced cardiac filling due to active decompression 402. As a consequence of the enhanced cardiac filling, cardiac output and pressure increases during the following compression-decompression cycle. Enhanced cardiac filling due to active decompression 402 is realized by improving the efficacy of filling the heart with blood during the active decompression portion of the decompression phase 114, regardless of the duration of the time period associated with active decompression. Meanwhile, the use of a duty cycle with a compression phase 112 that is shorter than the decompression phase 114 (e.g., a 40:60 duty cycle) by itself enhances cardiac filling by providing additional time to fill the heart 120 during the decompression phase 114, as compared to the allotted “filling time” using a 50:50 duty cycle, regardless of how the filling is accomplished during that time period.

The utilization of active decompression in combination with a duty cycle having a compression phase 112 that is shorter than the decompression phase 114 also enhances cardiac filling (improving cardiac output, pressure, circulation, and perfusion), but the benefits are realized in two different ways. Active decompression creates a negative pressure in the atria during the decompression phase 114, which sucks in more blood, thereby increasing the filling of the heart with blood. This effect is optimized during the time period of active decompression in the decompression phase 114 (e.g., a time period from about 0.39 seconds to the end of the compression-decompression cycle at 0.6 seconds). The duty cycle with a compression phase 112 that is shorter than the decompression phase 114, on the other hand, provides a longer decompression phase 114, which allows more blood to flow into the atria, thereby increasing the filling of the heart with blood. In other words, the duty cycle with a compression phase 112 that is shorter than the decompression phase 114 (e.g., a 40:60 duty cycle) increases (e.g., optimizes) the amount of time available for filling by increasing (e.g., maximizing) the time period of the decompression phase 114. These two effects complement each other, and the combination of active decompression and the duty cycle with a compression phase 112 that is shorter than the decompression phase 114 creates an unexpected positive synergistic effect, since they both optimize the same effect in different ways. Thus, the result of combining active decompression with a duty cycle having a compression phase 112 that is shorter than the decompression phase 114 has a greater effect and response, as compared a linear combination of active decompression followed by a duty cycle with a compression phase 112 that is shorter than the decompression phase 114, or vice versa. This positive synergistic effect is at least partially shown in FIG. 4 by the additional time in active decompression 404.

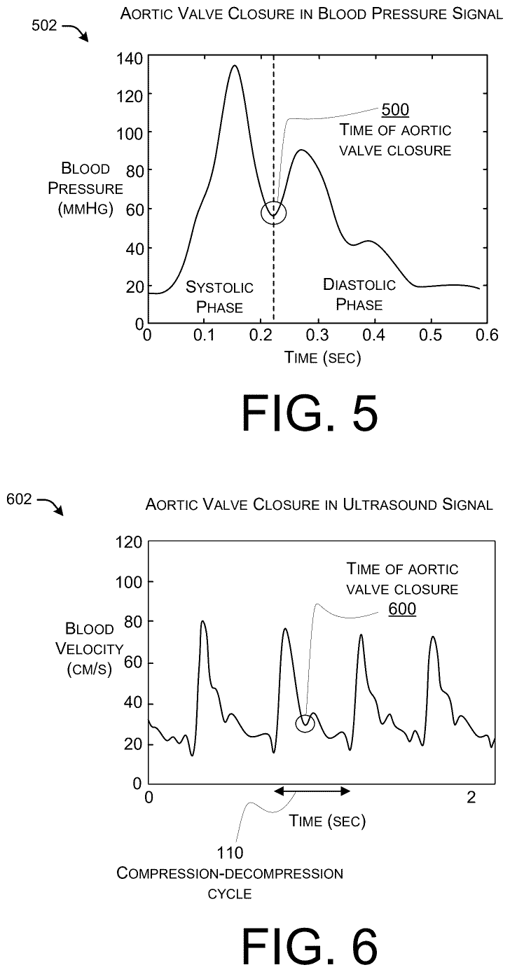

As noted above, closure of the aortic valve is one example criterion that is evaluated to determine the duty cycle of a CPR system and/or a CPR device. The aortic valve closure may be detected in several different physiological signals associated with the subject 104. FIGS. 5-8 illustrate various signals that can be analyzed for detecting closure of the aortic valve during CPR treatment of a subject 104. FIG. 5 illustrates a time of aortic valve closure 500 exhibited in a blood pressure signal 502. For example, the pulse-shaped, continuous blood pressure signal 502 associated with a subject 104 can be analyzed (while the subject 104 is receiving CPR treatment) to identify the dicrotic notch in the blood pressure signal 502. In FIG. 5, the dicrotic notch, and thus the time point at which the aortic valve closes 500, is illustrated in the blood pressure signal 502, which may represent a continuous arterial blood pressure signal. A reliable arterial continuous blood pressure signal can be obtained from an invasive measurement, such as an arterial line. Accordingly, in some examples, a processor(s) of a CPR system and/or CPR device (e.g., the processor(s) of the mechanical chest compression device 102, the processor(s) of the defibrillator 208, the processor(s) of the rescuer device 212, etc.) is configured to determine, by analyzing a blood pressure parameter(s) sensed by a sensor(s), that that an aortic valve of the heart of the subject 104 has closed, which is an example of a criterion being satisfied for implementing the techniques described herein.

FIG. 6 illustrates a time of aortic valve closure 600 exhibited in an ultrasound signal 602. The ultrasound signal 602 is another type of signal in which the dicrotic notch is visible and detectable. In FIG. 6, the dicrotic notch, and thus the time point at which the aortic valve closes 600, is illustrated in an ultrasound signal 602. The ultrasound signal is a non-invasive measurement, which is a particular advantage, as compared to, for example, a continuous arterial blood pressure signal or another invasive measurement. The pulse-shaped ultrasound signal 602 may be obtained in any blood vessel with a pulsatile flow. Thus, reliable detection of the time point for the closing of the aortic valve 600 may be obtained using an ultrasound signal 602. Accordingly, in some examples, a processor(s) of a CPR system and/or CPR device (e.g., the processor(s) of the mechanical chest compression device 102, the processor(s) of the defibrillator 208, the processor(s) of the rescuer device 212, etc.) is configured to determine, by analyzing an ultrasound parameter(s) (or a blood flow parameter(s)) sensed by a sensor(s), that that an aortic valve of the heart of the subject 104 has closed, which is an example of a criterion being satisfied for implementing the techniques described herein.

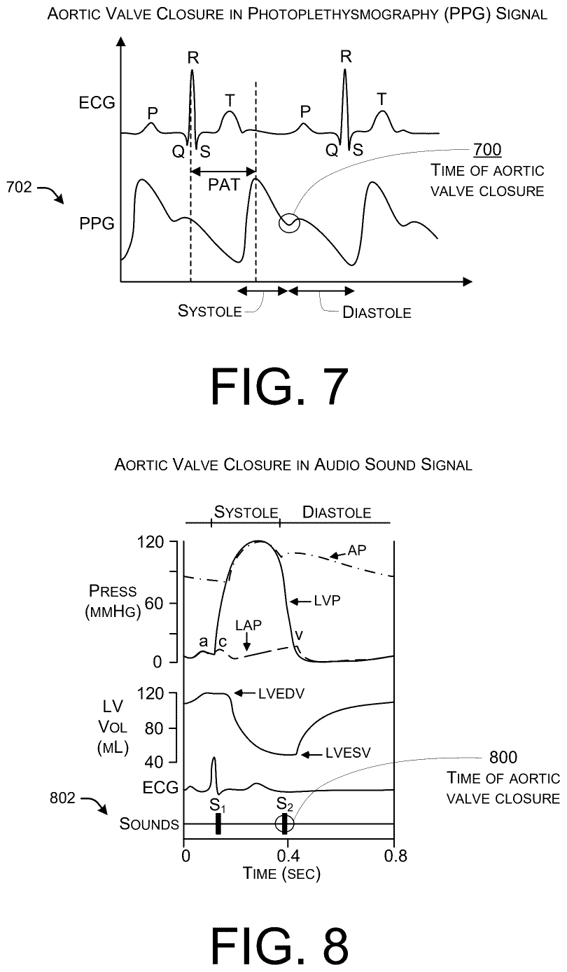

FIG. 7 illustrates a time of aortic valve closure 700 exhibited in a PPG signal 702. The PPG signal 702 is yet another physiological signal in which the dicrotic notch is visible and detectable. In FIG. 7, the dicrotic notch, and thus the time point at which the aortic valve closes 700, is illustrated in the PPG signal 702. The PPG signal 702 is also a non-invasive measurement, which is a particular advantage, as compared to, for example, a continuous arterial blood pressure signal or another invasive measurement. In some examples, the pulse-shaped PPG signal 702 may be obtained from a sensor(s) (e.g., a pulse oximeter) with access to any blood vessel with a pulsatile flow. Thus, a reliable detection of the time point for the closing of the aortic valve 700 may be obtained using a PPG signal 702. Accordingly, in some examples, a processor(s) of a CPR system and/or CPR device (e.g., the processor(s) of the mechanical chest compression device 102, the processor(s) of the defibrillator 208, the processor(s) of the rescuer device 212, etc.) is configured to determine, by analyzing a PPG parameter(s) sensed by a sensor(s), that that an aortic valve of the heart of the subject 104 has closed, which is an example of a criterion being satisfied for implementing the techniques described herein.

FIG. 8 illustrates a time of aortic valve closure 800 exhibited in an audio sound signal 802. Heart sound is a physiological signal that does not rely on analyzing a pulse-shaped signal or detecting the presence of a dicrotic notch in a pulse-shaped signal to determine whether the aortic valve has closed. The beating sound from a spontaneously beating heart is generated by the closing of the heart valves. The heart sound during one cardiac cycle consists of two beating sounds. The first, softer sound is generated by the closing of the mitral valve (and the closing of the tricuspid valve), and the second, louder sound is generated by the closing of the aortic valve (and the closing of the pulmonary valve). In FIG. 8, the two heart sounds, S₁ and S₂, during one cardiac cycle are illustrated in an audible sound signal 802, where the second beating sound, S₂, defines the time point for the closing of the aortic valve 800. The heart sound signal 802 is also a non-invasive measurement, which is a particular advantage, as compared to, for example, a continuous arterial blood pressure signal. An audible heart sound signal may be obtained from a microphone(s) (a type of sensor) and/or an auscultation device(s)/sensor(s) attached to, and/or placed on, the skin surface of the thorax (e.g., the chest or the back) of the subject 104. Thus, a reliable detection of the time point for the closing of the aortic valve 800 may be obtained using an audible sound signal 802 captured using a microphone(s) and/or an auscultation device(s)/sensor(s) during CPR. Accordingly, in some examples, a processor(s) of a CPR system and/or CPR device (e.g., the processor(s) of the mechanical chest compression device 102, the processor(s) of the defibrillator 208, the processor(s) of the rescuer device 212, etc.) is configured to determine, by analyzing an audible heart sound parameter(s) sensed by a sensor(s), that that an aortic valve of the heart of the subject 104 has closed, which is an example of a criterion being satisfied for implementing the techniques described herein.

As mentioned, the dicrotic notch in a pulse-shaped signal may be used to detect the time point of the closing of the aortic valve. However, in some instances, the dicrotic notch may not be visible in the signal, making it difficult to detect closure of the aortic valve via such a signal, at least in some patient populations (e.g., in patients with arterial stiffness). The pulse shape tends to be more triangular shaped the more arterial stiffness progresses, and in patients with severe arterial stiffness, the dicrotic notch is completely lacking. Thus, in some patient populations it is beneficial to detect the closing of the aortic valve using a physiological signal that does not rely on a detectable dicrotic notch, such as the audible sound signal 802 described above. There are other patient populations that may cause interference with the measurement of physiological signals, such as diabetes patients. Smaller blood vessels tend to be occluded in diabetes patients, which may result in poor perfusion. The poor perfusion in diabetes patients may influence the measurement of a PPG signal, and, in some cases, it may even make the detection of aortic valve closure impracticable.

Detection of closure of the aortic valve in real-time for every compression-decompression cycle may allow for optimizing the duty cycle used in association with a CPR system (e.g., a mechanical chest compression device 102). However, for real-time detection of the closing of the aortic valve, the transit time to the measurement point should be considered. Here, the transit time means the delay between the time at which the heart “beats” (due to CPR) and the time at which the resulting pulse is observable at the measurement location. If the delay is too long, that measurement may not be usable for real-time detection of aortic valve closure because the duty cycle adjustment must be made within the same compression-decompression cycle, which is about 0.6 seconds in duration. To illustrate, an audible heart sound in an audio sound signal 802 has the shortest delay time, and a PPG signal 702 measured at the fingers of the subject 104, for example, has the longest delay time, amongst the signals described with reference to FIGS. 5-8. An ultrasound signal 602 has a very short delay if the flow is measured at the aorta, and the same is true with a blood pressure signal 502. However, if ultrasound and blood pressure are measured farther away from the heart, then the transit time may be too long for a real-time detection of the closing of the aortic valve to be used in adjusting the duty cycle on-the-fly. In such examples, detecting aortic valve closure via the audible sound signal 802 has a particular advantage, as compared to using other physiological signals.

It is to be appreciated that, in addition to using the techniques described above for detecting closure of the aortic valve, other techniques can be used in combination with one or more of those techniques for purposes of detecting aortic valve closure. For example, detecting closure of the aortic valve may include the use of one or more detection and classification techniques, such as the use of adaptive filters, matched filters, time and frequency domain methods, correlation and cross-correlation methods, and/or artificial intelligence methods (e.g., machine learning models, including neural networks and the like).

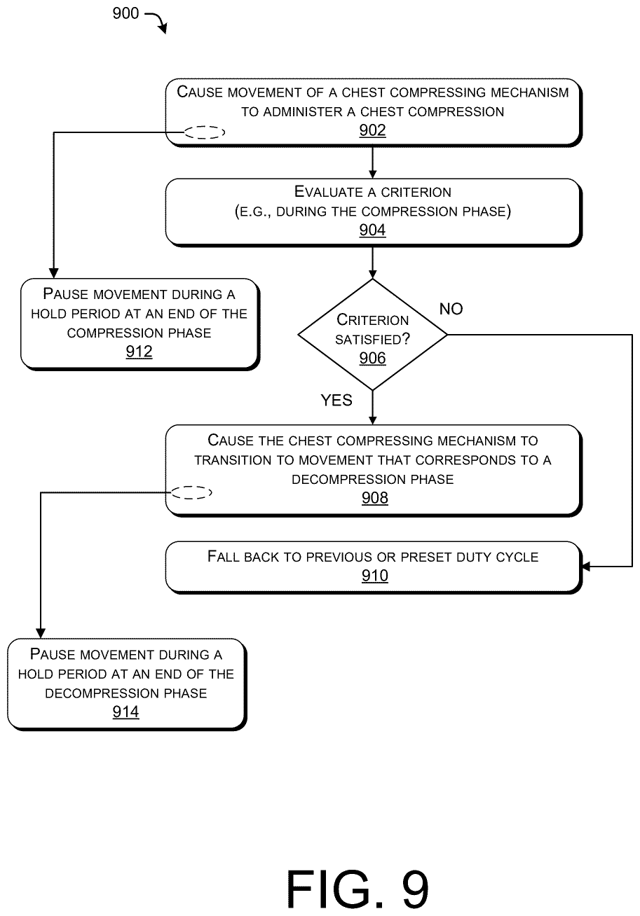

FIGS. 9, 10, 11, 12, 24, 25, 26A-26C, and 27 illustrate processes and/or techniques performed by one or more systems, devices, or entities described herein. For example, the processes/techniques illustrated in FIGS. 9, 10, 11, 12, 24, 25, 26A-26C, and 27 may be implemented by a CPR system and/or device, whether the device is a standalone device or part of a CPR system, and/or by at least one processor configured to execute instructions. The processes described herein represent sequences of operations that can be implemented in hardware, software, or a combination thereof. In the context of software, the blocks represent computer-executable instructions stored on one or more computer-readable storage media that, when executed by a processor(s), perform the recited operations. Generally, computer-executable instructions include routines, programs, objects, components, data structures, and the like that perform particular functions or implement particular abstract data types. The order in which the operations are described is not intended to be construed as a limitation, and any number of the described operations can be combined in any order and/or in parallel to implement the processes. In some examples, an operation(s) of the process may be omitted entirely. Moreover, the processes described herein can be combined in whole or in part with each other or with other processes.

FIG. 9 illustrates an example process 900 for controlling movement of a chest compressing mechanism of a mechanical chest compression device based on an evaluation of one or more criteria during CPR. The process 900 is performed by an entity including, for example, at least one of the mechanical chest compression device 102 of FIG. 1, the mechanical chest compression device 1400 of FIG. 14, a processor(s) of any of the aforementioned devices and/or a processor(s) of any other device(s) that is part of a CPR system.

At 902, the entity causes, during a compression phase 112 of a compression-decompression cycle 110, a chest compressing mechanism of a mechanical chest compression device to move for administering a chest compression to a subject 104. The compression-decompression cycle 110 may represent one cycle 110 of a series of compression-decompression cycles over which the chest compressing mechanism moves for administering chest compressions to the subject 104. The chest compressing mechanism is configured to be disposed on a chest of the subject 104. The type of movement of the chest compressing mechanism may depend on the type of mechanical chest compression device that is utilized for administering CPR treatment to the subject 104. For example, a piston-based mechanical chest compression device 102, as shown in FIG. 1, includes a chest compressing mechanism 106 in the form of a piston, and the piston is configured to be placed on the chest of the subject 104 and to repeatedly move downward, toward the subject 104, to apply a downward force to the chest for administering chest compressions to the subject 104 and to release the applied force by moving upward, away from the subject 104, in between successive applications of the downward force. As another example, a vest-based mechanical chest compression device includes a chest compressing mechanism in the form of a load-distributing band, and the load-distributing band is configured to be placed on the chest of the subject 104 and to repeatedly apply a distributed (squeezing) force that is directed inward towards the chest for administering chest compressions to the subject 104 and to release the applied force in between successive applications of the force.

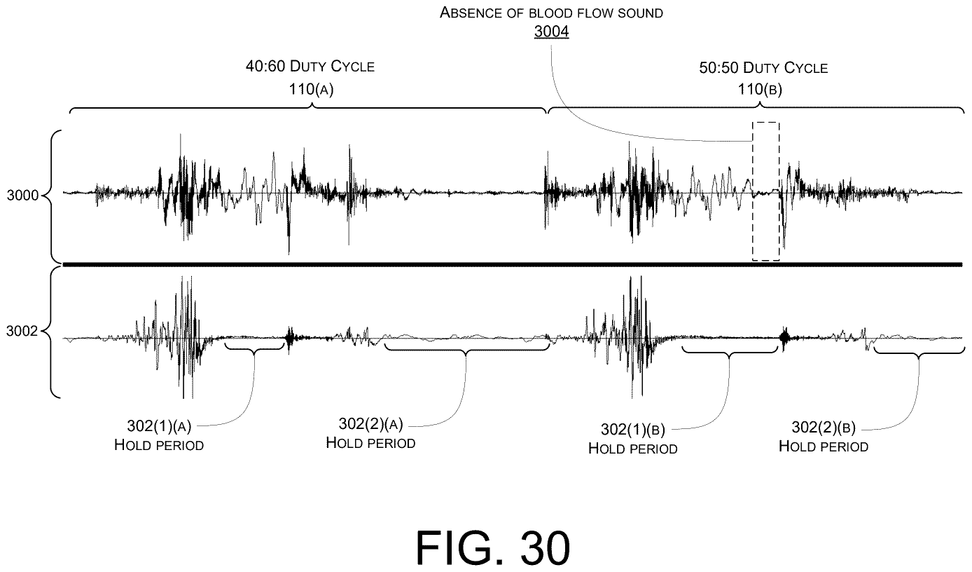

At 904, the entity evaluates a criterion (or criteria) to determine whether the criterion (or criteria) is satisfied at 906. In some examples, the criterion is evaluated at 904 during the compression phase 112. In some examples, determining whether the criterion is satisfied at 906 includes determining, during the compression phase 112, and by analyzing a parameter(s) associated with the subject 104 and sensed by a sensor(s), whether an aortic valve or a pulmonary valve of a heart of the subject 104 has closed. In some examples, this determination of closure of the aortic valve or the pulmonary valve occurs in real-time during the compression phase 112 of each compression-decompression cycle 110 of the series of compression-decompression cycles. The parameter(s) analyzed (at 904) for determining whether the aortic valve or the pulmonary valve has closed (at 906) can vary, as described above, and may include one or more of a blood pressure parameter(s) (See FIG. 5), a blood flow parameter(s) and/or an ultrasound parameter(s) (See FIG. 6), a PPG parameter(s) (See FIG. 7), and/or an audible heart sound parameter(s) (See FIG. 8). In some examples, an audible heart sound parameter(s) that is analyzed (at 904) to determine whether the aortic valve or the pulmonary valve has closed (at 906) can include the absence of a blood flow sound during the compression phase 112, as exhibited in an audio signal generated using an auscultation device(s) (See FIG. 30). Accordingly, in some examples, the duty cycle of the mechanical chest compression device is determined on-the-fly for each compression-decompression cycle 110 of the series of compression-decompression cycles by determining, during the compression phase 112 of each compression-decompression cycle, whether and when the aortic valve or the pulmonary valve has closed. As described above with reference to the waveform 108 depicted in FIG. 1, the expected time of aortic valve closure 118 is about 0.2 seconds into a compression-decompression cycle 110 having a duration of 0.6 seconds. Accordingly, if the closure of the aortic valve or the pulmonary valve is detected during the compression phase 112 and is used as a trigger event for transitioning to the decompression phase 114, the compression phase 112 is highly likely to be shorter than the decompression phase 114 for that compression-decompression cycle. In other words, the duty cycle that is determined on-the-fly for the current compression-decompression cycle 110 is likely to be a non-50:50 duty cycle representing a ratio of a compression time period to a decompression time period that is less than one, such as a 35:65 duty cycle, a 40:60 duty cycle, a 45:55 duty cycle, or any similar duty cycle with a compression phase 112 that is shorter than the decompression phase 114 of the compression-decompression cycle 110.

In a real-time implementation of detecting closure of the aortic valve or the pulmonary valve, the time at which the aortic valve or the pulmonary valve closes is detected in real-time for each compression-decompression cycle 110 over the series of compression-decompression cycles. Thus, the compression phases 112, decompression phases 114, and duty cycles for individual compression-decompression cycles may vary over time during ongoing CPR treatment. The detection and determination of the duty cycle for each compression-decompression cycle over time allowing the duty cycle of the mechanical chest compression device to vary over time, potentially cycle-to-cycle, and to be close to, if not match, the true optimal duty cycle for the subject 104 at any given time. Note, the “true optimal duty cycle” may be different over time for the same subject 104. The real-time implementation also ensures that the compression phase 112 does not end prematurely (e.g., before the closing of the aortic valve or the pulmonary valve), thus ensuring that the compression phases 112 are not too short, which prevents any counteracting back flow of blood into the heart from the aorta. A real-time implementation will thus have a time-varying duty cycle, potentially with different duty cycles for each compression-decompression cycle.

In some examples, determining whether the criterion is satisfied at 906 includes determining whether an amount of time since a start of the compression phase 112 has expired. In some examples, this amount of time is a predetermined amount of time that corresponds to a predetermined duty cycle, which represents a ratio of a first time period of the compression phase 112 to a second time period of the decompression phase 114. In other words, the mechanical chest compression device, in some examples, may be preprogramed with a fixed duty cycle, where the compression phase 112 is shorter than the decompression phase 114 for each compression-decompression cycle 110. For example, if the mechanical chest compression device is preprogrammed to utilize a 35:65 duty cycle at a compression rate of 100 compressions per minute, the criterion is satisfied at 906 at 0.21 seconds into each compression-decompression cycle 110. In some examples, the amount of time is a predetermined amount of time greater than an estimated time at which an aortic valve or a pulmonary valve of the heart of the subject will close after the start of the compression phase 112. In this example, the duty cycle that is selected for the mechanical chest compression device to use throughout a CPR session can be based on clinical data that is analyzed to determine the estimated time of closure of the aortic valve or the pulmonary valve for a patient population. For example, clinical data from multiple different patients over the course of multiple chest compressions for each patient may be collected and used to estimate a time of closure of the aortic valve or the pulmonary valve for a given patient population, which is then used to determine the fixed duty cycle of the mechanical chest compression device. In some examples, the clinical data is used to calculate a mean value of the time at which the aortic valve or the pulmonary valve closes for a patient population, as well as the standard deviation of the mean value. The standard deviation may be used to ensure that the compression phase 112 does not end before the closing of the aortic valve or the pulmonary valve, thereby ensuring that the compression phase 112 is not too short, which avoids counteracting back flow of blood into the heart from the aorta. In some examples, an amount of time (based at least in part on the standard deviation) is added to the mean time of closure of the aortic valve or the pulmonary valve, which effectively adds a safety margin (or cushion) to the mean value. In this manner, a duty cycle is determined with a conservative compression phase 112 that is not too short. This safety margin may create a compression phase 112 that is slightly too long for some subject receiving CPR treatment, but it ensures that the compression phase 112 is not too short for the vast majority of subjects. In some examples, instead of using clinical data to determine the fixed duty cycle of the mechanical chest compression device, the time of closure of the aortic valve or the pulmonary valve may be estimated, and the fixed duty cycle determined therefrom, based on theoretical calculations, data simulations, advanced mathematical simulations of CPR models, and/or laboratory experiments and tests. In this example, the estimated time of closure of the aortic valve or the pulmonary valve may be derived from physiological models and/or clinical data from several different patients over the course of multiple chest compressions. Additionally, or alternatively, preclinical (e.g., animal) data may also be used to estimate the time of closure of the aortic valve or the pulmonary valve, which, in turn, is used to determine the fixed duty cycle of the mechanical chest compression device. In some examples, instead of using a standard deviation to add a safety margin, as described above, any suitable amount of time may be added to the mean value of the estimated time of closure of the aortic valve or the pulmonary valve to provide a safety margin ensuring that the compression phase 112 is not too short, and/or a bias may be built into the mean value calculation of the time of closure of the aortic valve or the pulmonary valve, which results in a more conservative estimate of the time of valve closure.

The previous paragraph describes implementations of a fixed duty cycle where a timer (or clock) can be monitored (at 904) for each compression-decompression cycle to determine when (at 906) to transition to the decompression phase 114. However, a fixed duty cycle implementation does not vary the duty cycle on a cycle-to-cycle basis. In order to adapt the duty cycle to the subject 104 throughout a CPR session, the amount of time that is monitored (at 904) for each compression-decompression cycle can be periodically adjusted throughout the CPR session. For instance, the entity may analyze a parameter(s) associated with the subject 104 and sensed by a sensor(s) over a duration of multiple preceding compression-decompression cycles that precede the current compression-decompression cycle 110 to determine the amount of time to wait before the criterion is satisfied at 906. For example, the entity may analyze parameters sensed over multiple (e.g., the past two, five, ten, fifteen, twenty, thirty, etc.) preceding compression-decompression cycles to determine times at which the aortic valve or the pulmonary valve closed during the respective compression-decompression cycles, and the entity may calculate a statistic (e.g., a mean time of closure of the aortic valve or the pulmonary valve, a median time of closure of the aortic valve or the pulmonary valve, etc.) to determine how to adjust the duty cycle of the mechanical chest compression device. In this implementation, the parameter data may be collected over multiple preceding compression-decompression cycles, but the calculation and adjustment of the duty cycle, if needed, will occur periodically (e.g., every ten compression-decompression cycles, every fifteen compression-decompression cycles, etc.). Using parameter data collected over multiple compression-decompression cycles may result in a more robust and reliable detection of the time point for the aortic valve closure and/or the pulmonary valve closure, as compared to only using parameter data from a single compression-decompression cycle. However, this implementation is unable to adapt and change the duty cycle of the mechanical chest compression device as quickly as the real-time implementation discussed above. Nevertheless, this implementation of analyzing a parameter(s) sensed over multiple preceding compression-decompression cycles to determine how to adjust the duty cycle may still account for variations over time, and may change adaptively to a different duty cycle value over time during the CPR treatment, even though the adaptation of the duty cycle may not occur as fast as in the real-time implementation. In some examples, parameter data collected over multiple preceding compression-decompression cycles can be analyzed to calculate a mean value of the time point for aortic valve closure or pulmonary valve closure, as well as the standard deviation of the mean value. The standard deviation may be used to ensure that the compression phase 112 does not end before the closing of the aortic valve or the pulmonary valve, thereby ensuring that the compression phase 112 is not too short, which avoids counteracting back flow of blood into the heart from the aorta. In some examples, a time (based at least in part on the standard deviation) is added to the mean time of closure of the aortic valve or the pulmonary valve, which effectively adds a safety margin (or cushion) to the mean value to conservatively determine a duty cycle where the compression phase 112 is not too short. This safety margin may create a compression phase 112 that is slightly too long for some of the compression-decompression cycles, but this implementation is nevertheless a patient-specific implementation where the mean value and the standard deviation are customized for the particular subject 104 due to the use of a parameter(s) associated with the subject 104 (e.g., blood pressure, blood flow, ultrasound, PPG, audible heart sound, etc.) for determining the duty cycle. These mean and standard deviation values that are specific to the subject 104 may be time varying and/or updated during ongoing CPR treatment, depending on how many preceding compression-decompression cycles are used in the implementation. In summary, even though this implementation does not allow for a “cycle-to-cycle” adjustment of the duty cycle, the duty cycle adjustment can be made frequent enough to be close to the true optimal duty cycle.

In some examples, evaluating the criterion at 904 includes analyzing a parameter(s) associated with the subject 104 and sensed by a sensor(s). This parameter analysis can be done in real-time during the compression phase 112 of the current compression-decompression cycle 110 or over a duration of multiple compression-decompression cycles (e.g., multiple preceding compression-decompression cycles that precede the current compression-decompression cycle 110). In some examples, the parameter(s) analyzed at 904 includes one or more chest compression parameters, such as compression acceleration, compression velocity, compression force, compression depth, electrical current of the motor that is driving the chest compressing mechanism 106, rotational speed of the motor, and/or any combination thereof. For example, the entity may analyze a compression depth parameter at 904. For instance, a processor(s) of the mechanical chest compression device 102 may be configured to monitor the position (e.g., absolute or relative position) and/or a proxy for the position of the mechanism 106, such as the electrical current of the motor driving the chest compressing mechanism 106, data from an accelerometer, etc., and/or the movement of the chest compressing mechanism 106. In this example of monitoring compression depth, determining whether the criterion is satisfied at 906 includes determining, during the compression phase 112, that the chest compressing mechanism 106 has arrived at a position (e.g., negative 5.0 cm, 5.3 cm, 5.5 cm, 6.0 cm, etc.) that represents a target compression depth. In some examples, determining whether the criterion is satisfied at 906 includes determining whether compression acceleration and/or compression velocity is zero. As another example, the parameter(s) analyzed at 904 includes one or more treatment related parameters, such as movement of the chest, compression distance of the chest, compression force on the chest, an ECG parameter(s), a transthoracic impedance parameter(s), an EtCO₂ parameter(s), a blood oxygenation parameter(s), a blood pressure parameter(s), a blood flow parameter(s), a PPG parameter(s), an ultrasound parameter(s), an audible heart sound parameter(s), and/or any combination thereof. In some examples, the entity may compute a statistic (e.g., an average value(s) (or mean value(s))) based on the parameter(s) sensed over a duration of multiple preceding compression-decompression cycles, and determining whether the criterion is satisfied at 906 includes determining whether the statistic (e.g., average value of the parameter) satisfies a threshold.

If the criterion (or criteria) is satisfied at 906, the process 900 follows the YES route from 906 to 908. At 908, the entity causes the chest compressing mechanism to transition to movement that corresponds to the decompression phase 114 in response to determining that the criterion (or criteria) is satisfied. For example, the chest compressing mechanism may transition to moving upward, away from the subject 104 at 908, or the chest compressing mechanism may otherwise release the compressive force that is being applied to the chest. The transition to the decompression phase 114 at 908 may occur immediately after the criterion is satisfied, or there may be a delay between satisfaction of the criterion and the transition to the decompression phase 114 at 908. For example, the entity may be configured to wait a predefined time period after determining that the criterion is satisfied at 906 before causing the chest compressing mechanism to transition to movement that corresponds to the decompression phase 114 at 908. This predefined time period can be any suitable amount of time, such as 0.04 seconds, 0.05 seconds, 0.1 seconds, 0.15 seconds, 0.2 seconds, 0.25 seconds, or any other suitable time period. This predefined time period may mitigate the risk of ending the compression phase 112 prematurely, especially in implementations where the satisfaction of the criterion at 906 is based on an estimated time of closure of the aortic valve or the pulmonary valve. Accordingly, in some examples, at 908, the entity causes the chest compressing mechanism to transition to the movement that corresponds to the decompression phase 114 within a threshold amount of time after satisfaction of the criterion (e.g., within a threshold amount of time after determining that the aortic valve or the pulmonary valve has closed, within a threshold amount of time after determining that the chest compressing mechanism has arrived at a position representing a target compression depth, etc.).

If the criterion (or criteria) is not satisfied at 906 (e.g., within a timeout period), the process 900 follows the NO route from 906 to 910. At 910, the entity may implement a fallback operation(s). In some examples, the fallback operation may be to utilize a previously used duty cycle and/or a preset duty cycle to determine when to cause the chest compressing mechanism to transition to movement that corresponds to the decompression phase 114. For example, if the entity is unable to detect closure of the aortic valve or the pulmonary valve, or if the entity otherwise misses the detection of valve closure, the mechanical chest compression device may fallback to using a preset duty cycle or may continue using the most recently-used duty cycle, such as a 40:60 duty cycle, or some other duty cycle. In an example where determining whether the criterion is satisfied at 906 includes determining whether an amount of time since a start of the compression phase 112 has expired, the timeout period mentioned above can be set to be longer than the amount of time that, if expired, satisfies the criterion at 906. In practice, implementations of a fixed duty cycle where a timer (or clock) is monitored (at 904) for each compression-decompression cycle will result in satisfaction of the criterion (at 906) for every compression-decompression cycle, but a fallback operation(s) can still be in place in case of an anomaly (e.g., where the main software program malfunctions).

The process 900 relates to controlling movement of a chest compressing mechanism of a mechanical chest compression device in association with a particular duty cycle, where the particular duty cycle is selected such that an individual compression-decompression cycle 110 of a series of compression-decompression cycles includes a compression phase 112 that is shorter than a decompression phase 114 following the compression phase 112. Said another way, the particular duty cycle is selected such that the chest compressions are administered to the subject 104 at a ratio of a compression time period to a decompression time period that is less than one. For example, the compression phase 112 may be within a range of about 35% to about 45% of a duration of the compression-decompression cycle 110, and/or the chest compressing mechanism may administer the chest compressions to the subject 104 at a duty cycle within a range of about 35:65 to about 45:55, wherein the duty cycle represents a ratio of a first time period of a compression phase 112 to a second time period of a decompression phase 114.

As shown by sub-blocks 912 and 914, in some examples, movement of the chest compressing mechanism can be paused at one or more times during a compression-decompression cycle 110 in accordance with one or more hold periods 302 of the compression and decompression profile (e.g., the waveform 300). For example, at 912, the entity causes the chest compressing mechanism to refrain from moving during a hold period 302(1) at an end of the compression phase 112 such that the chest is not compressed any further during the hold period 302(1). Additionally, or alternatively, at 914, the entity causes the chest compressing mechanism to refrain from moving during a hold period 302(2) at an end of the decompression phase 114 such that the chest is not decompressed any further during the hold period 302(2). If multiple different hold periods 302 are used in an individual compression-decompression cycle 110, the hold periods 302 can be of different durations (See FIG. 3), since the individual compression-decompression cycle 110 includes a compression phase 112 that is shorter than the decompression phase 114. As described above, these hold periods 302 may be beneficial when implemented in combination with such a duty cycle, as the hold period 302(1) at the end of the compression phase 112 allows the aortic valve to close before the compression phase 112 ends, and the hold period 302(2) at the end of the decompression phase 114 allows the heart to be filled with blood in a natural and undisturbed manner. In some examples, the chest compressing mechanism includes a piston with a suction cup disposed on a distal end of the piston, and a portion of the decompression phase 114 is associated with active decompression (See FIG. 4). In an active decompression implementation, the entity may, at 902, cause the piston to move from a first position to a second position during the compression phase 112, and may, at 908 (or at 910), cause the piston to move from the second position past the first position to a third position during the decompression phase 114, thereby actively decompressing the chest during movement of the piston from the first position to the third position as the suction cup pulls upward on the chest.

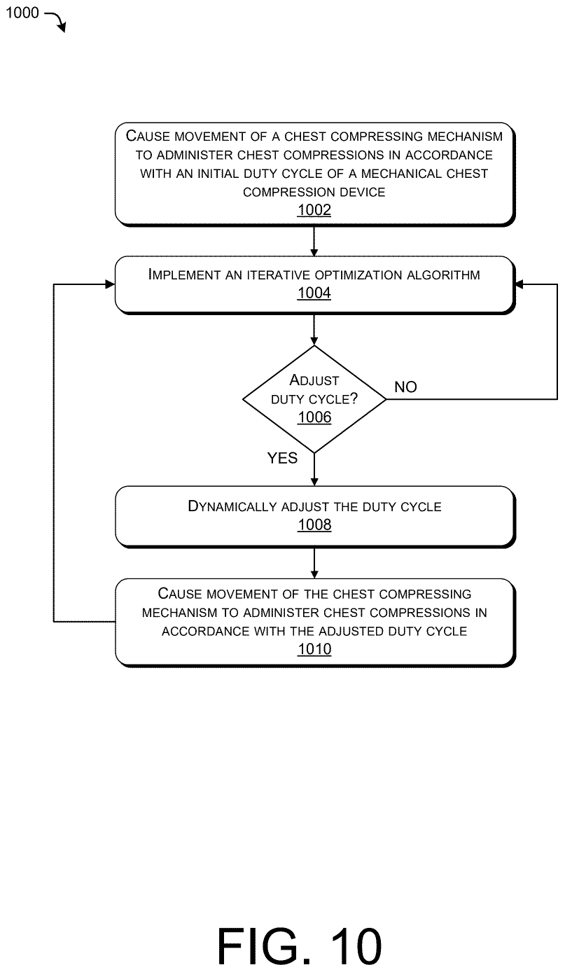

FIG. 10 illustrates an example process 1000 for implementing an iterative optimization algorithm to dynamically adjust a duty cycle of a mechanical chest compression device. The process 1000 is performed by an entity including, for example, at least one of the mechanical chest compression device 102 of FIG. 1, the mechanical chest compression device 1400 of FIG. 14, a processor(s) of any of the aforementioned devices and/or a processor(s) of any other device(s) that is part of a CPR system.

At 1002, the entity causes movement of a chest compressing mechanism of a mechanical chest compression device to administer chest compressions to a subject 104 in accordance with an initial duty cycle of the mechanical chest compression device. The initial duty cycle may be any suitable duty cycle including a 50:50 duty cycle, or a non-50:50 duty cycle where the compression phase 112 is shorter than the decompression phase 114 of the individual compression-decompression cycles. The initial duty cycle represents a ratio of a first time period of the compression phase 112 to a second time period of the decompression phase 114.

At 1004, the entity implements (or utilizes) an iterative optimization algorithm as the chest compressing mechanism administers the chest compressions to the subject 104 over a series of compression-decompression cycles. In some examples, implementation of the iterative optimization algorithm at 1004 includes selecting a candidate duty cycle that is different than the current duty cycle (e.g., the initial duty cycle used at 1002). After selecting the candidate duty cycle, the entity may cause the mechanical chest compression device to oscillate between the current (e.g., initial) duty cycle and the selected candidate duty cycle while analyzing a parameter(s) associated with the subject 104 over a predetermined number of compression-decompression cycles and/or for a predetermined time period. In some examples, the oscillation can switch the duty cycle every compression-decompression cycle, every two compression-decompression cycles, every three compression-decompression cycles, and so on. The parameter(s) analyzed at 1004 may be any suitable parameter(s), such as movement of the chest, compression distance of the chest, compression force on the chest, an ECG parameter(s), a transthoracic impedance parameter(s), an EtCO₂ parameter(s), a blood oxygenation parameter(s), a blood pressure parameter(s), a blood flow parameter(s), a PPG parameter(s), an ultrasound parameter(s), an audible heart sound parameter(s), and/or any combination thereof. In some examples, an audible heart sound parameter(s) that is analyzed (at 1004) the absence of a blood flow sound during the compression phase 112, as exhibited in an audio signal generated using an auscultation device(s) (See FIG. 30). Consider an example where the initial duty cycle used at 1002 is a 50:50 duty cycle, and the candidate duty cycle selected at 1004 is a 45:55 duty cycle. In this example, the entity causes the mechanical chest compression device to oscillate between the two different duty cycles while analyzing the parameter(s) associated with the subject 104 for a predetermined number of compression-decompression cycles and/or for a predetermined time period. Based on the analysis of the parameter(s) associated with each of the different duty cycles, the entity selects the better of the two duty cycles. For example, the entity may determine, based on the parameter analysis, that a first blood pressure parameter(s) associated with the 45:55 duty cycle is within a first value range (which may be indicative of a stable or improving condition of the subject 104), whereas a second blood pressure parameter(s) associated with the 50:50 duty cycle is within a second value range (which may be indicative of the an unstable or degrading condition of the subject 104). In this scenario, the entity selects the 45:55 duty cycle as being more favorable than (or preferred over) the 50:50 duty cycle. As another example, the entity may determine, based on the parameter analysis, that an audio signal generated using an auscultation device(s) exhibits an absence of blood flow sound at the end of the compression phase 112 associated with the 50:50 duty cycle, whereas the blood flow sound is present during the entirety of the compression phase 112 associated with a 45:55 duty cycle. In this scenario, the entity selects the 45:55 duty cycle as being more favorable than (or preferred over) the 50:50 duty cycle.

At 1006, the entity determines whether to adjust the duty cycle of the mechanical chest compression device based on the result of implementing the iterative optimization algorithm. If the entity determines to refrain from adjusting the duty cycle, the process 1000 follows the NO route from 1006 back to 1004, where the iterative optimization algorithm is implemented again at 1004. In some examples, the entity may wait for a predefined period of time before implementing the iterative optimization algorithm again at 1004. For instance, the entity may implement the iterative optimization algorithm every two minutes, three minutes, four minutes, five minutes, etc. following a determination to refrain from adjusting the duty cycle. In an illustrative example, if the result of the parameter analysis at 1004 results in selecting the current (e.g., initial) duty cycle (e.g., the 50:50 duty cycle in the example above), the entity may not adjust the duty cycle and may continue using the current (e.g., initial) duty cycle. In this example, the entity may wait for the predefined period of time before implementing the iterative optimization algorithm again at 1004.

If the entity determines, at 1006, to adjust the duty cycle, the process 1000 follows the YES route from 1006 to 1008. In the example above, if the entity selects the candidate duty cycle over the current (e.g., initial) duty cycle, the determination at 1006 may be to adjust the duty cycle from the current (e.g., initial) duty cycle to the selected candidate duty cycle. At 1008, the entity dynamically adjusts the duty cycle of the mechanical chest compression device. That is, the duty cycle is adjusted, or changed, as the chest compressions are being administered by the mechanical chest compression device so that the adjusted duty cycle can be used in the next compression-decompression cycle. In the example above, the entity may dynamically adjust the 50:50 duty cycle to the 45:55 duty cycle at 1008.

At 1010, the entity causes movement of the chest compressing mechanism to administer chest compressions to the subject 104 in accordance with the adjusted duty cycle of the mechanical chest compression device. In the example above, the adjusted duty cycle may be the 45:55 duty cycle where the compression phase 112 is shorter than the decompression phase 114 for an individual compression-decompression cycle. As shown by the return arrow from 1010 back to 1004, the iterative optimization algorithm can be implemented again at 1004, this time with a new candidate duty cycle. That is, the entity causes the mechanical chest compression device to oscillate between the adjusted duty cycle and the new candidate duty cycle while analyzing the parameter(s) associated with the subject 104 over the predetermined number of compression-decompression cycles and/or for a predetermined time period. For instance, if the adjusted duty cycle (which is now the current duty cycle) is a 45:55 duty cycle, the new candidate duty cycle may be a 40:60 duty cycle, and the entity determines which duty cycle to select based on the parameter analysis described above, and if the new candidate duty cycle is selected, the process 1000 iterates blocks 1008 and 1010 to adjust the duty cycle again. Following an adjustment of the duty cycle at 1008, the process 1000 may continue implementing the iterative optimization algorithm at 1004 until an optimal duty cycle is identified and used for at least a period of time before implementing the iterative optimization algorithm again. In some examples, the iterative optimization algorithm represents a gradient descent algorithm, or another suitable algorithm to iteratively optimize the duty cycle of the mechanical chest compression device.

It is to be appreciated that the entity, as part of the parameter analysis performed at 1004, may be configured to identify a pattern (or trend) in the analyzed parameters as the entity causes the mechanical chest compression device to oscillate between two duty cycles. In these examples, once the evaluation of the parameter(s) associated with a candidate duty cycle changes significantly (e.g., by diverging from the pattern (or trend) by more than a threshold amount), this may be taken as an indication that the candidate duty cycle is worse than the current duty cycle, and possibly worse than the previously selected candidate duty cycles. At this point, the entity may determine that the current duty cycle is an optimal duty cycle, and the entity may wait for a period of time before implementing the iterative optimization algorithm again at 1004 to re-evaluate the current duty cycle at a later time during the CPR session. It is also to be appreciated that the difference between the current duty cycle and the candidate duty cycle under evaluation at 1004 should be significant (e.g., greater than a threshold difference) to distinguish the performance of one duty cycle from the other duty cycle. In other words, if a 50:50 duty cycle is evaluated against a 49:51 duty cycle, the difference between the parameter(s) that are evaluated for each duty cycle may be insignificant, and, in that case, it may be too difficult to discern which duty cycle is better. It is also to be appreciated that artificial intelligence (e.g., a machine learning model(s)) may be used to evaluate the parameter(s) at 1004 and to select the better of the two duty cycles to determine, at 1006, whether to adjust the duty cycle at or not.

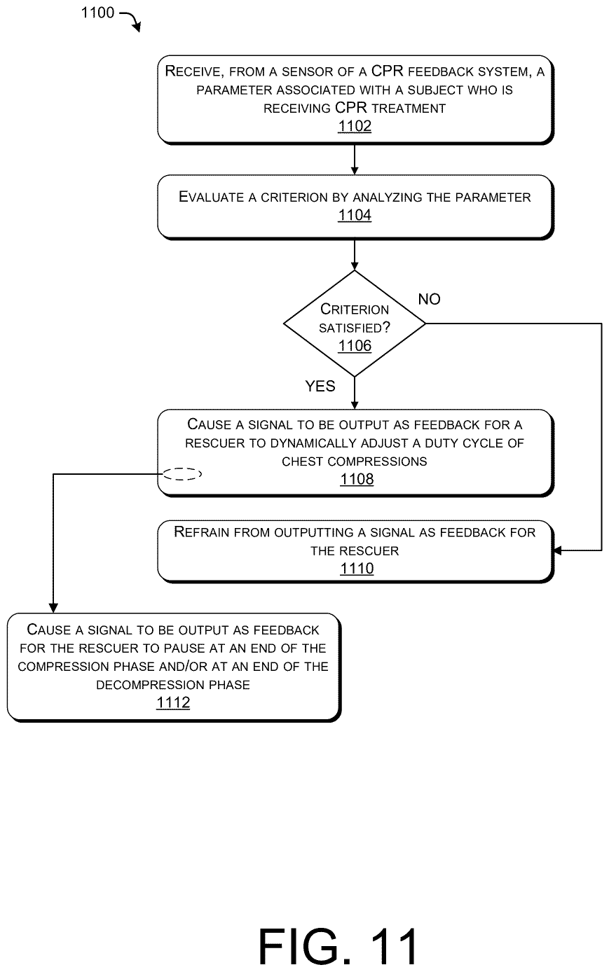

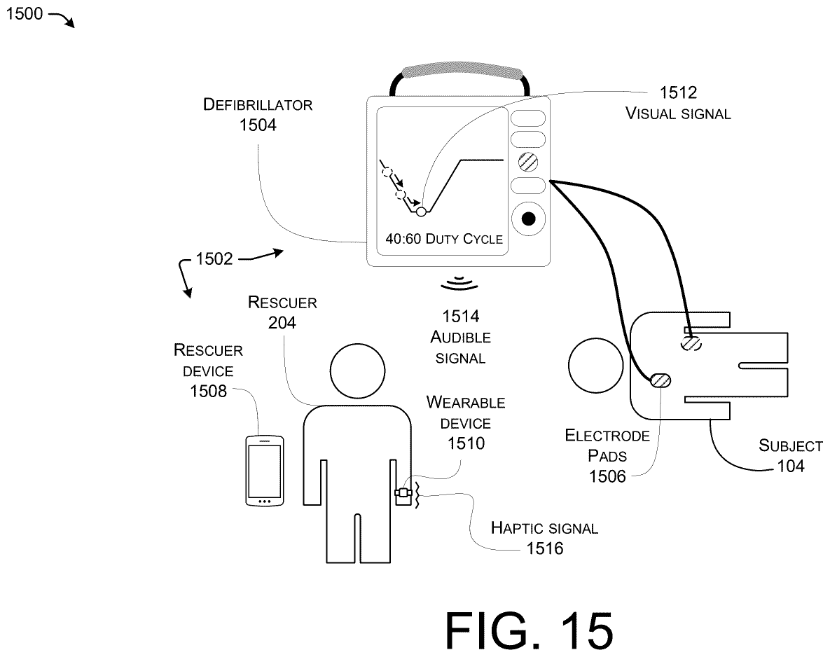

FIG. 11 illustrates an example process 1100 for providing, via a CPR feedback system, feedback for a rescuer 204 to dynamically adjust a duty cycle of chest compressions that the rescuer 204 is manually administering to a subject 104. The process 1100 is performed by an entity including, for example, one or more devices of a CPR duty cycle guidance system (e.g., the CPR feedback system 202 of FIG. 2, or the CPR duty cycle guidance system 1502 of FIG. 15), such as the defibrillator 208 of FIG. 2, the defibrillator 1300 of FIG. 13, the defibrillator 1504 of FIG. 15, the rescuer device 212 of FIG. 2, the rescuer device 1508 of FIG. 15, the wearable device 1510 of FIG. 15, the CPR feedback device 206 of FIG. 2, a processor(s) of any of the aforementioned devices and/or a processor(s) of any other device(s) that is part of a CPR system.

At 1102, the entity receives, from a sensor(s) of a CPR feedback system, a parameter(s) associated with a subject 104 as a rescuer 204 is manually administering chest compressions to the subject 104. For example, the subject 104 may be receiving CPR treatment administered by the rescuer 204 while a sensor(s) is sensing a parameter(s) associated with the subject 104. In some examples, the sensor(s) is configured to sense any suitable parameter(s) associated with subject 104 as a rescuer 204 is manually administering the chest compressions to the subject 104, such as movement of the chest, compression distance of the chest, compression force on the chest, an ECG parameter(s), a transthoracic impedance parameter(s), an EtCO₂ parameter(s), a blood oxygenation parameter(s), a blood pressure parameter(s), a blood flow parameter(s), an ultrasound parameter(s), a PPG parameter(s), an audible heart sound parameter(s). In some examples, the sensor(s) is configured to sense the parameter(s) over a duration of multiple compression-decompression cycles (e.g., two compression-decompression cycles, five compression-decompression cycles, ten compression-decompression cycles, twenty compression-decompression cycles, thirty compression-decompression cycles, etc.) and/or over a predefined time period (e.g., two seconds, five seconds, seven seconds, ten seconds, fifteen seconds, twenty seconds, etc.). In some examples, the sensor(s) is configured to be disposed on, or in proximity to, a chest of the subject 104. In some examples, the sensor(s) may be a sensor(s) 214 of the CPR feedback device 206 of FIG. 2, and the CPR feedback device 206 can be disposed on the chest of the subject 104 underneath the hands of the rescuer 204, on the back of the hands of the rescuer 204, on the chest of the subject 104 next to the hands of the rescuer 204, or in any other suitable position near or on the chest of the subject 104. In some examples, the sensor(s) 214 is a microphone or auscultation device(s)/sensor(s) configured to be disposed on a thorax of the subject 104, and the parameter(s) sensed by the sensor(s) is an audible heart sound parameter (See FIG. 8).

At 1104, the entity evaluates a criterion (or criteria) by analyzing the parameter(s) received from the sensor(s) to determine whether the criterion (or criteria) is satisfied at 1106. In some examples, the entity computes an average value of the parameter(s) sensed over the duration of the multiple compression-decompression cycles, and the entity determines that the criterion is satisfied at 1106 based at least in part on the average value of the parameter(s). For example, at 1104, audible heart sound parameters, such as the times of closure of the aortic valve of the heart of the subject 104 detected in an audible sound signal 802 (See FIG. 8), can be averaged over the multiple compression-decompression cycles and/or over the predefined time period in order to estimate a time of closure of the aortic valve or the pulmonary valve based on the average value of the audible heart sound parameters, and a duty cycle (and/or a compression phase duration, a decompression phase duration, etc.) can be determined based on the time estimate of the closure of the aortic valve or the pulmonary valve. In this example, the criterion may be satisfied at 1106 if the chest compressions being administered by the rescuer 204 are not in alignment with the determined duty cycle (and/or the determined compression phase duration, decompression phase duration, etc.). For example, the sensor(s) 214 of the CPR feedback device 206 may sense the compression rate, the compression time period, the decompression time period, and/or the like, as the rescuer 204 is manually administering the chest compressions, and if the average compression time period of the rescuer's compressions is greater than the compression phase duration determined based on the parameter analysis (e.g., for the determined duty cycle) by at least a threshold amount, and/or if the average decompression time period of the rescuer's compressions is less than the decompression phase duration determined based on the parameter analysis (e.g., for the determined duty cycle) by at least a threshold amount, the criterion may be satisfied at 1106. In some examples, an audible heart sound parameter(s) that is analyzed (at 1104) to determine whether the aortic valve or the pulmonary valve has closed (at 1106) can include the absence of a blood flow sound during the compression phase 112, as exhibited in an audio signal generated using an auscultation device(s) (See FIG. 30).

If the criterion (or criteria) is satisfied at 1106, the process 1100 follows the YES route from 1106 to 1108. At 1108, in response to determining that the criterion (or criteria) is satisfied, the entity causes a signal(s) to be output as feedback for the rescuer 204 to dynamically adjust a duty cycle of the chest compressions. In some examples, the feedback targets a first time period of the compression phase to be within a range of about 35% to about 45% of a duration of the compression-decompression cycle. In some examples, the signal(s) output at 1108 includes an audible signal(s), a visual signal(s), or a haptic signal(s). In some examples, the signal(s) output at 1108 includes a repeating audible signal(s), a repeating visual signal(s), and/or a repeating haptic signal(s) that represents a pace for the rescuer 204 to follow while manually administering the chest compressions. In some examples, the signal(s) output at 1108 includes an audible signal(s) and/or a visual signal(s) that prompts the rescuer 204 to wait longer between successive chest compressions. In some examples, the signal(s) is output via an output device of the defibrillator 208 (e.g., a monitor-defibrillator) communicatively coupled with the sensor(s) from which the parameter(s) was received at 1102. For example, the defibrillator 208 may be communicatively coupled with the sensor(s) 214 of the CPR feedback device 206 and/or with the CPR feedback device 206 itself. In some examples, the entity is configured to select a pattern from a set of predefined patterns, and the signal(s) that is output at 1108 is based on the selected pattern

If the criterion (or criteria) is not satisfied at 1106, the process 1100 follows the NO route from 1106 to 1110. At 1110, the entity refrains from outputting a signal(s) as feedback for the rescuer 204. For example, failure to satisfy the criterion (or criteria) may indicate that the chest compressions that the rescuer 204 is manually administering are in alignment with the determined duty cycle, as described above. For instance, as the sensor(s) 214 of the CPR feedback device 206 senses the compression rate, the compression time period, the decompression time period, and/or the like, if the average compression time period of the rescuer's compressions is within a threshold of the compression phase duration determined based on the parameter analysis (e.g., for the determined duty cycle), and/or if the average decompression time period of the rescuer's compressions is within a threshold of the decompression phase duration determined based on the parameter analysis (e.g., for the determined duty cycle), the criterion may not be satisfied at 1106, and the entity refrains from providing feedback to the rescuer 204 at 1110. Alternatively, the entity may cause a signal(s) to be output as a confirmation that the rescuer 204 is performing CPR properly (e.g., a message may be displayed on a display that reads “Good Job!”, “Nice Compressions!”, or the like).

At 1112, in some examples, the feedback for the rescuer 204 is further to pause at an end of the compression phase before transitioning to movement that corresponds to the decompression phase. Additionally, or alternatively, at 1112, in some examples, the feedback for the rescuer 204 is further to pause at an end of the decompression phase before transitioning to movement that corresponds to the compression phase of a next compression-decompression cycle. The feedback to pause at the end of either or both phases of the compression-decompression cycle may correspond to the hold periods 302(1) and/or 302(2) exhibited in the waveform 300 of FIG. 3, and described in more detail above.

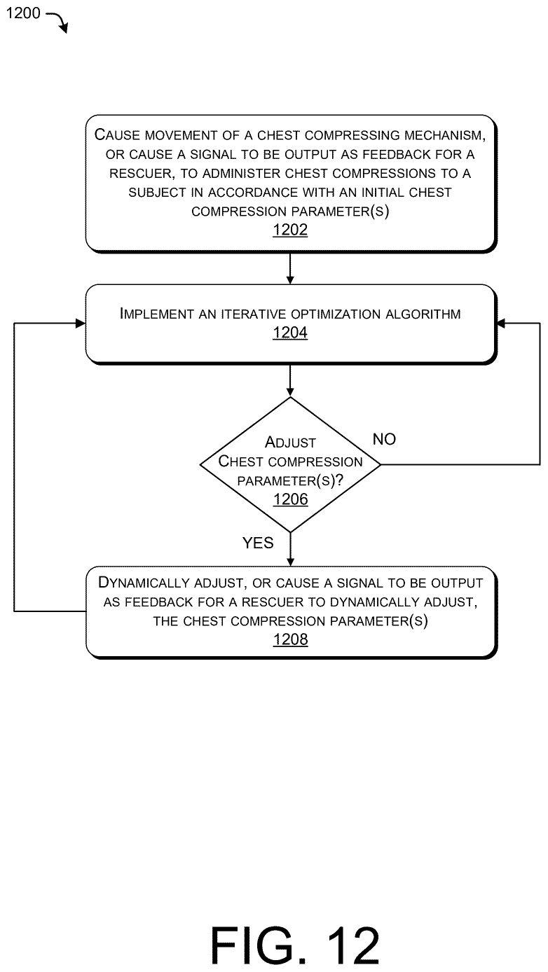

FIG. 12 illustrates an example process 1200 for implementing an iterative optimization algorithm to dynamically adjust one or more chest compression parameters associated with CPR treatment. The process 1200 is performed by an entity including, for example, one or more devices of a CPR system, such as the mechanical chest compression device 102 of FIG. 1, the mechanical chest compression device 1400 of FIG. 14, one or more devices of the CPR feedback system 202 of FIG. 2, such as the defibrillator 208 of FIG. 2, the defibrillator 1300 of FIG. 13, the rescuer device 212 of FIG. 2, the CPR feedback device 206 of FIG. 2, one or more devices of the CPR duty cycle guidance system 1502 of FIG. 15, such as the defibrillator 1504, the rescuer device 1508, or the wearable device 1510, a processor(s) of any of the aforementioned devices and/or a processor(s) of any other device(s) that is part of a CPR system.

At 1202, the entity causes movement of a chest compressing mechanism of a mechanical chest compression device, or causes a signal(s) to be output as feedback for the rescuer 204, to administer chest compressions to a subject 104 in accordance with an initial chest compression parameter(s). The initial chest compression parameter(s) can be any suitable type of parameter(s), such as compression depth, compression rate, compression force, position of the compression on the chest (e.g., the position of the chest compressing mechanism 106, hand position of the rescuer 204, etc.), a duty cycle of the chest compressions, and/or any combination thereof.

At 1204, the entity implements (or utilizes) an iterative optimization algorithm as the (manual or automated) chest compressions are being administered to the subject 104 over a series of compression-decompression cycles. In some examples, implementation of the iterative optimization algorithm at 1204 includes selecting a candidate chest compression parameter(s) that is different than the current chest compression parameter (e.g., the initial chest compression parameter(s) used at 1202). In some examples, the current (e.g., initial) chest compression parameter(s) and the selected candidate chest compression parameter(s) may be the same type of chest compression parameter, but different values of the parameter. For example, the current (e.g., initial) chest compression parameter may be a first compression rate (e.g., 100 compressions per minute) and the selected candidate chest compression parameter may be a second compression rate (e.g., 110 compressions per minute). Compression rate is just one example type of chest compression parameter that can be selected at 1204, and it is to be appreciated that other types of chest compression parameters can be selected in lieu of, or in combination with, compression rate.

After selecting the candidate chest compression parameter(s), the entity may cause the mechanical chest compression device or the rescuer 204 to oscillate between the current (e.g., initial) chest compression parameter(s) and the selected candidate chest compression parameter(s) while analyzing a parameter(s) associated with the subject 104 over a predetermined number of compression-decompression cycles and/or for a predetermined time period. In some examples, the oscillation can switch between the two different chest compression parameters every compression-decompression cycle, every two compression-decompression cycles, every three compression-decompression cycles, and so on. In a manual CPR example, the entity may cause a signal(s) to be output as feedback for the rescuer 204 to oscillate between the two different chest compression parameters. For example, if the signal(s) is output as a repeating audible signal(s), a repeating visual signal(s), and/or a repeating haptic signal(s) that represents a pace for the rescuer 204 to follow while manually administering the chest compressions, the signal(s) may oscillate between the two different compression rates (or between two different patterns corresponding to the two different compression rates) so that the rescuer 204 can follow along with the signal(s) being output to oscillate between the two different chest compression parameters. The parameter(s) analyzed at 1204 may be any suitable parameter(s), such as movement of the chest, compression distance of the chest, compression force on the chest, an ECG parameter(s), a transthoracic impedance parameter(s), an EtCO₂ parameter(s), a blood oxygenation parameter(s), a blood pressure parameter(s), a blood flow parameter(s), a PPG parameter(s), an ultrasound parameter(s), an audible heart sound parameter(s), and/or any combination thereof. Consider an example where the current (e.g., initial) chest compression parameter is a first compression rate of 100 compressions per minute, and the selected candidate chest compression parameter is a second compression rate of 110 compressions per minute. In this example, the entity analyzes the parameter(s) associated with the subject 104 for a predetermined number of compression-decompression cycles and/or for a predetermined time period while the mechanical chest compression device or the rescuer 204 is oscillating between the two different compression rates. Based on the analysis of the parameter(s) associated with each of the two different chest compression parameters, the entity selects the better of the two chest compression parameters. For example, the entity may determine, based on the parameter analysis, that a first blood pressure parameter(s) associated with the current (e.g., initial) compression rate is within a first value range (which may be indicative of a stable or improving condition of the subject 104), whereas a second blood pressure parameter(s) associated with the selected candidate compression rate is within a second value range (which may be indicative of the an unstable or degrading condition of the subject 104). In this scenario, the entity selects the current (e.g., initial) compression rate (e.g., 100 compressions per minute) as being more favorable than (or preferred over) the selected candidate compression rate (e.g., 110 compressions per minute).

At 1206, the entity determines whether to the chest compression parameter(s) should be adjusted based on the result of implementing the iterative optimization algorithm. If the entity determines that the chest compression parameter should not be adjusted, the process 1200 follows the NO route from 1206 back to 1204, where the iterative optimization algorithm is implemented again at 1204. In some examples, the entity may wait for a predefined period of time before implementing the iterative optimization algorithm again at 1204. For instance, the entity may implement the iterative optimization algorithm every two minutes, three minutes, four minutes, five minutes, etc. following a determination to refrain from adjusting the chest compression parameter(s). In an illustrative example, if the result of the parameter analysis at 1204 results in selecting current (e.g., initial) chest compression parameter, the entity may determine, at 1206, that the chest compression parameter(s) should not be adjusted that the mechanical chest compression device or the rescuer 204 should continue using the current (e.g., initial) duty cycle. In this example, the entity may wait for the predefined period of time before implementing the iterative optimization algorithm again at 1204,

If the entity determines, at 1206, to adjust the chest compression parameter(s), the process 1200 follows the YES route from 1206 to 1208. In the example above, if the entity selects the candidate chest compression parameter(s) over the current (e.g., initial) chest compression parameter(s), the determination at 1206 may be that the chest compression parameter(s) should be adjusted from the current (e.g., initial) chest compression parameter(s) to the selected candidate chest compression parameter(s). At 1208, the entity dynamically adjusts, or causes a signal(s) to be output as feedback for the rescuer 204 to dynamically adjust, the chest compression parameter(s) in association with the CPR treatment that the subject 104 is receiving. That is, the chest compression parameter(s) is adjusted, or the signal(s) is output, as the chest compressions are being administered by the mechanical chest compression device or the rescuer 204 so that the adjusted chest compression parameter(s) can be used in the next compression-decompression cycle and/or the upcoming compression-decompression cycles. In the example above, the entity may dynamically adjust the compression rate used by the mechanical chest compression device to the selected candidate compression rate (e.g., 110 compressions per minute) at 1208. Alternatively, the entity may cause a signal(s) (e.g., an audible signal(s), a visual signal(s), a haptic signal(s), etc.) to be output as feedback for the rescuer 204 to dynamically adjust the compression rate to the selected candidate compression rate (e.g., 110 compressions per minute) at 1208. Again, although compression rate is provided as an example chest compression parameter that is adjusted at 1208, the chest compression parameter(s) adjusted at 1208 may be any suitable chest compression parameter, such as compression depth, compression rate, compression force, position of the compression on the chest (e.g., the position of the chest compressing mechanism 106, hand position of the rescuer 204, etc.), a duty cycle of the chest compressions, and/or any combination thereof.

In an example where automated chest compressions are being administered via a mechanical chest compression device, the entity, at 1208, may cause movement of the chest compressing mechanism to administer chest compressions to the subject 104 in accordance with the adjusted chest compression parameter(s) of the mechanical chest compression device.

As shown by the return arrow from 1208 back to 1204, the iterative optimization algorithm can be implemented again at 1204, this time with a new candidate chest compression parameter(s). That is, the entity causes the mechanical chest compression device or the rescuer 204 to oscillate between the adjusted chest compression parameter(s) and the new candidate chest compression parameter(s) while analyzing the parameter(s) associated with the subject 104 over the predetermined number of compression-decompression cycles and/or for a predetermined time period. For instance, if the adjusted chest compression parameter(s) (which is now the current chest compression parameter(s)) is a first compression rate of 110 compressions per minute, the new candidate compression rate may be a second compression rate of 120 compressions per minute, and the entity determines which compression rate to select based on the parameter analysis described above, and if the new candidate compression rate is selected, the process 1200 iterates block 1208 to adjust the chest compression parameter(s) again. Following an adjustment of the chest compression parameter(s) at 1208, the process 1200 may continue implementing the iterative optimization algorithm at 1204 until an optimal chest compression parameter(s) is identified and used for at least a period of time before implementing the iterative optimization algorithm again. In some examples, the iterative optimization algorithm represents a gradient descent algorithm, or another suitable algorithm to iteratively optimize the chest compression parameter(s) associated with the CPR treatment.

It is to be appreciated that the entity, as part of the parameter analysis performed at 1204, may be configured to identify a pattern (or trend) in the analyzed parameters as the entity causes the mechanical chest compression device or the rescuer 204 to oscillate between two chest compression parameters (or two sets of chest compression parameters). In these examples, once the evaluation of the parameter(s) associated with a candidate chest compression parameter(s) changes significantly (e.g., by diverging from the pattern (or trend) by more than a threshold amount), this may be taken as an indication that the candidate chest compression parameter(s) is worse than the current chest compression parameter(s), and possibly worse than the previously selected candidate chest compression parameter(s). At this point, the entity may determine that the current chest compression parameter(s) is an optimal chest compression parameter(s), and the entity may wait for a period of time before implementing the iterative optimization algorithm again at 1204 to re-evaluate the current chest compression parameter(s) at a later time during the CPR session. It is also to be appreciated that the difference between the current chest compression parameter(s) and the candidate chest compression parameter(s) under evaluation at 1204 should be significant (e.g., greater than a threshold difference) to distinguish the performance of one chest compression parameter(s) from the other chest compression parameter(s). In other words, if, say, first compression depth of negative 5 cm is evaluated against a second compression depth of negative 5.1 cm, the difference between the parameter(s) that are evaluated for each compression depth parameter may be insignificant, and, in that case, it may be too difficult to discern which chest compression parameter(s) is better. It is also to be appreciated that artificial intelligence (e.g., a machine learning model(s)) may be used to evaluate the parameter(s) at 1204 and to select the better of the two chest compression parameter(s) to determine, at 1206, whether to adjust the chest compression parameter(s) or not.

FIG. 13 illustrates an example of an external defibrillator 1300 configured to perform various functions described herein. For example, the external defibrillator 1300 is the defibrillator 208 described above with reference to FIG. 2.

The external defibrillator 1300 includes an electrocardiogram (ECG) port 1302 connected to multiple ECG wires 1304. In some cases, the ECG wires 1304 are removeable from the ECG port 1302. For instance, the ECG wires 1304 are plugged into the ECG port 1302 via connectors. The ECG wires 1304 are connected to ECG electrodes 1306, respectively. In various implementations, the ECG electrodes 1306 are disposed on different locations on an individual 1308. For example, the individual 1308 is the subject 104 described above. A detection circuit 1310 is configured to detect relative voltages between the ECG electrodes 1306. These voltages are indicative of the electrical activity of the heart of the individual 1308.

In various implementations, the ECG electrodes 1306 are in contact with the different locations on the skin of the individual 1308. In some examples, a first one of the ECG electrodes 1306 is placed on the skin between the heart and right arm of the individual 1308, a second one of the ECG electrodes 1306 is placed on the skin between the heart and left arm of the individual 1308, and a third one of the ECG electrodes 1306 is placed on the skin between the heart and a leg (either the left leg or the right leg) of the individual 1308. In these examples, the detection circuit 1310 is configured to measure the relative voltages between the first, second, and third ECG electrodes 1306. Respective pairings of the ECG electrodes 1306 are referred to as “leads,” and the voltages between the pairs of ECG electrodes 1306 are known as “lead voltages.” In some examples, more than three ECG electrodes 1306 are included, such that 5-lead or 12-lead ECG signals are detected by the detection circuit 1310.

The detection circuit 1310 includes at least one analog circuit, at least one digital circuit, or a combination thereof. The detection circuit 1310 receives the analog electrical signals from the ECG electrodes 1306, via the ECG port 1302 and the ECG wires 1304. In some cases, the detection circuit 1310 includes one or more analog filters configured to filter noise and/or artifact from the electrical signals. The detection circuit 1310 includes an analog-to-digital converter (ADC) in various examples. The detection circuit 1310 generates a digital signal indicative of the analog electrical signals from the ECG electrodes 1306. This digital signal can be referred to as an “ECG signal” or an “ECG.”

In some cases, the detection circuit 1310 further detects an electrical impedance between at least one pair of the ECG electrodes 1306. For example, the detection circuit 1310 includes, or otherwise controls, a power source that applies a known voltage (or current) across a pair of the ECG electrodes 1306 and detects a resultant current (or voltage) between the pair of the ECG electrodes 1306. In various cases, the current is applied via a high-frequency (e.g., 20 kHz) carrier signal. The impedance is generated based on the applied signal (voltage or current) and the resultant signal (current or voltage). In various cases, the impedance corresponds to respiration of the individual 1308, chest compressions performed on the individual 1308, and other physiological states of the individual 1308. In various examples, the detection circuit 1310 includes one or more analog filters configured to filter noise and/or artifact from the resultant signal. The detection circuit 1310 generates a digital signal indicative of the impedance using an ADC. This digital signal can be referred to as an “impedance signal” or an “impedance.”

The detection circuit 1310 provides the ECG signal and/or the impedance signal to one or more processors 1312 in the external defibrillator 1300. In some implementations, the processor(s) 1312 includes a central processing unit (CPU), a graphics processing unit (GPU), both CPU and GPU, or other processing unit or component known in the art.

The processor(s) 1312 is operably connected to memory 1314. In various implementations, the memory 1314 is volatile (such as random access memory (RAM)), non-volatile (such as read only memory (ROM), flash memory, etc.) or some combination of the two. The memory 1314 stores instructions that, when executed by the processor(s) 1312, causes the processor(s) 1312 to perform various operations. In various examples, the memory 1314 stores methods, threads, processes, applications, objects, modules, any other sort of executable instruction, or a combination thereof. In some cases, the memory 1314 stores files, databases, or a combination thereof. In some examples, the memory 1314 includes, but is not limited to, RAM, ROM, electrically erasable programmable read-only memory (EEPROM), flash memory, or any other memory technology. In some examples, the memory 1314 includes one or more of compact disc-ROMs (CD-ROMs), digital versatile discs (DVDs), content-addressable memory (CAM), or other optical storage, magnetic cassettes, magnetic tape, magnetic disk storage or other magnetic storage devices, or any other medium which can be used to store the desired information and which can be accessed by the processor(s) 1312 and/or the external defibrillator 1300. In some cases, the memory 1314 at least temporarily stores the ECG signal and/or the impedance signal.

In various examples, the memory 1314 includes a detector 1316, which causes the processor(s) 1312 to determine, based on the ECG signal and/or the impedance signal, whether the individual 1308 is exhibiting a particular heart rhythm. For instance, the processor(s) 1312 determines whether the individual 1308 is experiencing a shockable rhythm that is treatable by defibrillation. Examples of shockable rhythms include ventricular fibrillation (VF) and ventricular tachycardia (V-Tach). In some examples, the processor(s) 1312 determines whether any of a variety of different rhythms (e.g., asystole, sinus rhythm, atrial fibrillation (AF), etc.) are present in the ECG signal.

The processor(s) 1312 is operably connected to one or more input devices 1318 and one or more output devices 1320. Collectively, the input device(s) 1318 and the output device(s) 1320 function as an interface between a user and the defibrillator 1300. The input device(s) 1318 is configured to receive an input from a user and includes at least one of a keypad, a cursor control, a touch-sensitive display, a voice input device (e.g., a microphone), a haptic feedback device (e.g., a gyroscope), or any combination thereof. The output device(s) 1320 includes at least one of a display, a speaker, a haptic output device, a printer, or any combination thereof. In various examples, the processor(s) 1312 causes a display among the output device(s) 1320 to visually output a waveform of the ECG signal and/or the impedance signal. In some implementations, the input device(s) 1318 includes one or more touch sensors, the output device(s) 1320 includes a display screen, and the touch sensor(s) are integrated with the display screen. Thus, in some cases, the external defibrillator 1300 includes a touchscreen configured to receive user input signal(s) and visually output physiological parameters, such as the ECG signal and/or the impedance signal.

In some examples, the memory 1314 includes an advisor 1322, which, when executed by the processor(s) 1312, causes the processor(s) 1312 to generate advice and/or control the output device(s) 1320 to output the advice to a user (e.g., a rescuer). In some examples, the processor(s) 1312 provides, or causes the output device(s) 1320 to provide, an instruction to perform CPR on the individual 1308. In some cases, the processor(s) 1312 evaluates, based on the ECG signal, the impedance signal, or other physiological parameters, CPR being performed on the individual 1308 and causes the output device(s) 1320 to provide feedback about the CPR in the instruction. According to some examples, the processor(s) 1312, upon identifying that a shockable rhythm is present in the ECG signal, causes the output device(s) 1320 to output an instruction and/or recommendation to administer a defibrillation shock to the individual 1308.

The memory 1314 also includes an initiator 1324 which, when executed by the processor(s) 1312, causes the processor(s) 1312 to control other elements of the external defibrillator 1300 in order to administer a defibrillation shock to the individual 1308. In some examples, the processor(s) 1312 executing the initiator 1324 selectively causes the administration of the defibrillation shock based on determining that the individual 1308 is exhibiting the shockable rhythm and/or based on an input from a user (received, e.g., by the input device(s) 1318). In some cases, the processor(s) 1312 causes the defibrillation shock to be output at a particular time, which is determined by the processor(s) 1312 based on the ECG signal and/or the impedance signal.

In various cases, the memory 1314 includes a CPR component 1327 configured to operate in association with a particular chest compression parameter(s), such as a particular duty cycle, associated with chest compressions being administered manually by a rescuer. In some cases, the CPR component 1327, when executed by the processor(s) 1312, causes the defibrillator 1300 to pair with and/or authenticate the CPR feedback device 206 and/or the rescuer device 212 of FIG. 2, cause a signal(s) to be output as feedback for a rescuer to dynamically adjust a chest compression parameter(s) (e.g., a duty cycle), associated with chest compressions being administered manually by a rescuer, and/or to perform the techniques and/or processes described herein, such as the process 1100, the process 1200, and/or any other technique or process described herein.

The processor(s) 1312 is operably connected to a charging circuit 1323 and a discharge circuit 1325. In various implementations, the charging circuit 1323 includes a power source 1326, one or more charging switches 1328, and one or more capacitors 1330. The power source 1326 includes, for instance, a battery. The processor(s) 1312 initiates a defibrillation shock by causing the power source 1326 to charge at least one capacitor among the capacitor(s) 1330. For example, the processor(s) 1312 activates at least one of the charging switch(es) 1328 in the charging circuit 1323 to complete a first circuit connecting the power source 1326 and the capacitor to be charged. Then, the processor(s) 1312 causes the discharge circuit 1325 to discharge energy stored in the charged capacitor across a pair of defibrillation electrodes 1334, which are in contact with the individual 1308. For example, the processor(s) 1312 deactivates the charging switch(es) 1328 completing the first circuit between the capacitor(s) 1330 and the power source 1326, and activates one or more discharge switches 1332 completing a second circuit connecting the charged capacitor 1330 and at least a portion of the individual 1308 disposed between defibrillation electrodes 1334. In various cases, more than two defibrillation electrodes 1334 are disposed on the skin of the individual 1308, which define multiple shock vectors.

The energy is discharged from the defibrillation electrodes 1334 in the form of a defibrillation shock. For example, the defibrillation electrodes 1334 are connected to the skin of the individual 1308 and located at positions on different sides of the heart of the individual 1308, such that the defibrillation shock is applied across the heart of the individual 1308. The defibrillation shock, in various examples, depolarizes a significant number of heart cells in a short amount of time. The defibrillation shock, for example, interrupts the propagation of the shockable rhythm (e.g., VF or VT) through the heart. In some examples, the defibrillation shock is 200 J or greater with a duration of about 0.015 seconds. In some cases, the defibrillation shock has a multiphasic (e.g., biphasic) waveform. The discharge switch(es) 1332 are controlled by the processor(s) 1312, for example. In various implementations, the defibrillation electrodes 1334 are connected to defibrillation wires 1336. The defibrillation wires 1336 are connected to a defibrillation port 1338, in implementations. According to various examples, the defibrillation wires 1336 are removable from the defibrillation port 1338. For example, the defibrillation wires 1336 are plugged into the defibrillation port 1338.

In various implementations, the processor(s) 1312 is operably connected to one or more transceivers 1340 that transmit and/or receive data over one or more communication networks 1342. For example, the transceiver(s) 1340 includes a network interface card (NIC), a network adapter, a local area network (LAN) adapter, or a physical, virtual, or logical address to connect to the various external devices and/or systems. In various examples, the transceiver(s) 1340 includes any sort of wireless transceivers capable of engaging in wireless communication (e.g., radio frequency (RF) communication). For example, the communication network(s) 1342 includes one or more wireless networks that include a 3^{rd} Generation Partnership Project (3GPP) network, such as a Long Term Evolution (LTE) radio access network (RAN) (e.g., over one or more LTE bands), a New Radio (NR) RAN (e.g., over one or more NR bands), or a combination thereof. In some cases, the transceiver(s) 1340 includes other wireless modems, such as a modem for engaging in WI-FI®, WIGIG®, WIMAX®, BLUETOOTH®, or infrared communication over the communication network(s) 1342.

The defibrillator 1300 is configured to transmit and/or receive data (e.g., ECG data, impedance data, data indicative of one or more detected heart rhythms of the individual 1308, data indicative of one or more defibrillation shocks administered to the individual 1308, data associated with CPR treatment the individual 1308 is receiving, etc.) with one or more external devices 1344 via the communication network(s) 1342. The external devices 1344 include, for instance, mobile devices (e.g., mobile phones, smart watches, etc.), Internet of Things (IoT) devices, medical devices, computers (e.g., laptop devices, servers, etc.), or any other type of computing device configured to communicate over the communication network(s) 1342. In some examples, the external device(s) 1344 is located remotely from the defibrillator 1300, such as at a remote clinical environment (e.g., a hospital). According to various implementations, the processor(s) 1312 causes the transceiver(s) 1340 to transmit data to the external device(s) 1344. In some cases, the transceiver(s) 1340 receives data from the external device(s) 1344 and the transceiver(s) 1340 provide the received data to the processor(s) 1312 for further analysis.

In various implementations, the external defibrillator 1300 also includes a housing 1346 that at least partially encloses other elements of the external defibrillator 1300. For example, the housing 1346 encloses the detection circuit 1310, the processor(s) 1312, the memory 1314, the charging circuit 1323, the transceiver(s) 1340, or any combination thereof. In some cases, the input device(s) 1318 and output device(s) 1320 extend from an interior space at least partially surrounded by the housing 1346 through a wall of the housing 1346. In various examples, the housing 1346 acts as a barrier to moisture, electrical interference, and/or dust, thereby protecting various components in the external defibrillator 1300 from damage.

In some implementations, the external defibrillator 1300 is an automated external defibrillator (AED) operated by an untrained user (e.g., a bystander, layperson, etc.) and can be operated in an automatic mode. In automatic mode, the processor(s) 1312 automatically identifies a rhythm in the ECG signal, makes a decision whether to administer a defibrillation shock, charges the capacitor(s) 1330, discharges the capacitor(s) 1330, or any combination thereof. In some cases, the processor(s) 1312 controls the output device(s) 1320 to output (e.g., display) a simplified user interface to the untrained user. For example, the processor(s) 1312 refrains from causing the output device(s) 1320 to display a waveform of the ECG signal and/or the impedance signal to the untrained user, in order to simplify operation of the external defibrillator 1300.

In some examples, the external defibrillator 1300 is a monitor-defibrillator utilized by a trained user (e.g., a clinician, an emergency responder, etc.) and can be operated in a manual mode or the automatic mode. When the external defibrillator 1300 operates in manual mode, the processor(s) 1312 cause the output device(s) 1320 to display a variety of information that may be relevant to the trained user, such as waveforms indicating the ECG data and/or impedance data, notifications about detected heart rhythms, and the like.

FIG. 14 illustrates a mechanical chest compression device 1400 configured to perform various functions described herein. For example, the mechanical chest compression device 1400 is the mechanical chest compression device 102 described in FIG. 1.

In various implementations, the mechanical chest compression device 1400 includes a compressor 1402 that is operatively coupled to a motor 1404. For example, the compressor 1402 is the chest compressing mechanism 106 described in FIG. 1. The compressor 1402 physically administers a force to the chest of a subject 1406 that compresses the chest of the subject 1406. For example, the subject 1406 is the subject 104 described in FIG. 1. In some examples, the compressor 1402 includes at least one piston 1405 that periodically moves between two or more positions (e.g., a compressed position and a release position) at a compression frequency. For example, when the piston 1405 is positioned on the chest of the subject 1406, the piston 1405 compresses the chest when the piston 1405 is moved into the compressed position. A suction cup 1407 may be positioned on a tip of the piston 1405, such that the suction cup 1407 contacts the chest of the subject 1406 during operation. In various cases, the compressor 1402 includes a band (e.g., a load-distributing band) that periodically tightens to a first tension and loosens to a second tension at a compression frequency. For instance, when the band is disposed around the chest of the subject 1406, the band compresses the chest when the band tightens.

The motor 1404 is configured to convert electrical energy stored in a power source 1408 into mechanical energy that moves and/or tightens the compressor 1402, thereby causing the compressor 1402 to administer the force to the chest of the subject 1406. In various implementations, the power source 1408 is portable. For instance, the power source 1408 includes at least one rechargeable (e.g., lithium-ion) battery. In some cases, the power source 1408 supplies electrical energy to one or more elements of the mechanical chest compression device 1400 described herein.

In various cases, the mechanical chest compression device 1400 includes a support 1410 that is physically coupled to the compressor 1402, such that the compressor 1402 maintains a position relative to the subject 1406 during operation. In some implementations, the support 1410 is physically coupled to a backplate 1412, cot, or other external structure with a fixed position relative to the subject 1406. In some examples, the support 1410 includes a pair of legs that are configured to connect to the backplate 1412 at respective sides of the backplate 1412. According to some cases, the support 1410 is physically coupled to a portion of the subject 1406, such as wrists of the subject 1406.

The operation of the mechanical chest compression device 1400 may be controlled by at least one processor 1414. In various implementations, the motor 1404 is communicatively coupled to the processor(s) 1414. Specifically, the processor(s) 1414 is configured to output a control signal to the motor 1404 that causes the motor 1404 to actuate the compressor 1402. For instance, the motor 1404 causes the compressor 1402 to administer the compressions to the subject 1406 based on the control signal. In some cases, the control signal indicates one or more treatment parameters (sometimes referred to herein as “chest compression parameters”) of the compressions. Examples of treatment parameters include a frequency, timing, depth, force, position, velocity, and acceleration of the compressor 1402 administering the compressions. According to various cases, the control signal causes the motor 1404 to cease compressions and/or to hold the compressor 1402 in a fixed position for a hold period 302.

In various implementations, the mechanical chest compression device 1400 includes at least one transceiver 1416 configured to communicate with at least one external device 1418 over one or more communication networks 1420. Any communication network described herein can be included in the communication network(s) 1420 illustrated in FIG. 14. The external device(s) 1418, for example, includes at least one of a monitor-defibrillator, an AED, an ECMO device, a ventilation device, a patient monitor, a mobile phone, a server, or a computing device. In some implementations, the transceiver(s) 1416 is configured to communicate with the external device(s) 1418 by transmitting and/or receiving signals wirelessly. For example, the transceiver(s) 1416 includes a NIC, a network adapter, a LAN adapter, or a physical, virtual, or logical address to connect to the various external devices and/or systems. In various examples, the transceiver(s) 1416 includes any sort of wireless transceivers capable of engaging in wireless communication (e.g., RF communication). For example, the communication network(s) 1420 includes one or more wireless networks that include a 3GPP network, such as an LTE RAN (e.g., over one or more LTE bands), an NR RAN (e.g., over one or more NR bands), or a combination thereof. In some cases, the transceiver(s) 1416 includes other wireless modems, such as a modem for engaging in WI-FI®, WIGIG®, WIMAX®, BLUETOOTH®, or infrared communication over the communication network(s) 1420. The signals, in various cases, encode data in the form of data packets, datagrams, or the like. In some cases, the signals are transmitted as compressions are being administered by the mechanical chest compression device 1400 (e.g., for real-time feedback by the external device(s) 1418), after compressions are administered by the mechanical chest compression device 1400 (e.g., for post-event review at the external device 1418), or a combination thereof.

In various cases, the processor(s) 1414 generates the control signal based on data encoded in the signals received from the external device(s) 1418. For instance, the signals include an instruction to initiate the compressions, and the processor(s) 1414 instructs the motor 1404 to begin actuating the compressor 1402 in accordance with the signals.

In some cases, the mechanical chest compression device 1400 includes at least one input device 1422. In various examples, the input device(s) 1422 is configured to receive an input signal from a user 1424, who may be a rescuer treating the subject 1406. Examples of the input device(s) 1422 include, for instance, a keypad, a cursor control, a touch-sensitive display, a voice input device (e.g., a microphone), a haptic feedback device (e.g., a gyroscope), or any combination thereof. In various implementations, the processor(s) 1414 generates the control signal based on the input signal. For instance, the processor(s) 1414 generates the control signal to adjust a frequency of the compressions based on the mechanical chest compression device 1400 detecting a selection by the user 1424 of a user interface element displayed on a touchscreen or detecting the user 1424 pressing a button integrated with an external housing of the mechanical chest compression device 1400.

According to some examples, the input device(s) 1422 include one or more sensors. The sensor(s), for example, is configured to detect a physiological parameter of the subject 1406. In some implementations, the sensor(s) is configured to detect a state parameter of the mechanical chest compression device 1400, such as a position of the compressor 1402 with respect to the subject 1406 or the backplate 1412, a force administered by the compressor 1402 on the subject 1406, a force administered onto the backplate 1412 by the body of the subject 1406 during a compression, or the like. According to some implementations, the signals transmitted by the transceiver(s) 1416 indicate the physiological parameter(s) and/or the state parameter(s). In some examples, the sensor(s) include a microphone(s) and/or an auscultation device(s)/sensor(s) configured to capture an audible sound signal 802 (e.g., an audible heart sound) during CPR. For instance, the sensor(s) are disposed on surfaces of the backplate 1412, the compressor 1402 (e.g., the distal end of the suction cup 1407), or a combination thereof.

The mechanical chest compression device 1400 further includes at least one output device 1425, in various implementations. Examples of the output device(s) 1425 include, for instance, least one of a display (e.g., a projector, an LED screen, etc.), a speaker, a haptic output device, a printer, a light emitting element(s) (LED), or any combination thereof. In some implementations, the output device(s) 1425 include a screen configured to display various parameters detected by and/or reported to the mechanical chest compression device 1400, a charge level of the power source 1408, a timer indicating a time since compressions were initiated or paused, and other relevant information.

The mechanical chest compression device 1400 further includes memory 1426. In various implementations, the memory 1426 is volatile (such as random access memory (RAM)), non-volatile (such as read only memory (ROM), flash memory, etc.) or some combination of the two. The memory 1426 stores instructions that, when executed by the processor(s) 1414, causes the processor(s) 1414 to perform various operations. In various examples, the memory 1426 stores methods, threads, processes, applications, objects, modules, any other sort of executable instruction, or a combination thereof. In some cases, the memory 1426 stores files, databases, or a combination thereof. In some examples, the memory 1426 includes, but is not limited to, RAM, ROM, EEPROM, flash memory, or any other memory technology. In some examples, the memory 1426 includes one or more of CD-ROMs, DVDs, CAM, or other optical storage, magnetic cassettes, magnetic tape, magnetic disk storage or other magnetic storage devices, or any other medium which can be used to store the desired information. In various cases, the memory 1426 stores instructions, programs, threads, objects, data, or any combination thereof, that cause the processor(s) 1414 to perform various functions. In various cases, the memory 1426 stores one or more parameters that are detected by the mechanical chest compression device 1400 and/or reported to the mechanical chest compression device 1400.

In various cases, the memory 1426 includes a CPR component 1427 configured to operate in association with a particular chest compression parameter(s), such as a particular duty cycle, associated with automated chest compressions administered by the mechanical chest compression device 1400. In some cases, the CPR component 1327, when executed by the processor(s) 1312, causes the compressor 1402 to move for administering the chest compressions to the subject 1406 over a series of compression-decompression cycles, wherein a compression-decompression cycle 110 of the series of compression-decompression cycles includes a compression phase 112 that is shorter than a decompression phase 114. In some examples, the CPR component 1327, when executed by the processor(s) 1312, further causes the processor(s) 1312 to determine, during the compression phase 112, that a criterion is satisfied, and to cause the compressor 1402 to transition to movement that corresponds to the decompression phase 114 in response to determining that the criterion is satisfied, and/or to perform the techniques and/or processes described herein, such as the process 900, the process 1000, the process 1200, and/or any other technique or process described herein. In some examples, a treatment circuit 1430 is used to administer treatment via the mechanical chest compression device 1400.

FIG. 15 illustrates an environment 1500 including an example CPR duty cycle guidance system 1502 that is configured to provide duty cycle guidance to a rescuer 204 who is manually administering chest compressions to the subject 104. As compared to the CPR feedback system 202 depicted in FIG. 2, the CPR duty cycle guidance system 1502 depicted in FIG. 15 omits the CPR feedback device 206. In some examples, the CPR duty cycle guidance system 1502 includes a defibrillator 1504, which may be the same as, or similar to, the defibrillator 208 introduced in FIG. 2. Various example components of the defibrillator 1504 shown in FIG. 15 are described in more detail above with respect to FIG. 13. In some examples, the rescuer 204 operates the defibrillator 1504 to monitor and potentially treat the subject 104.

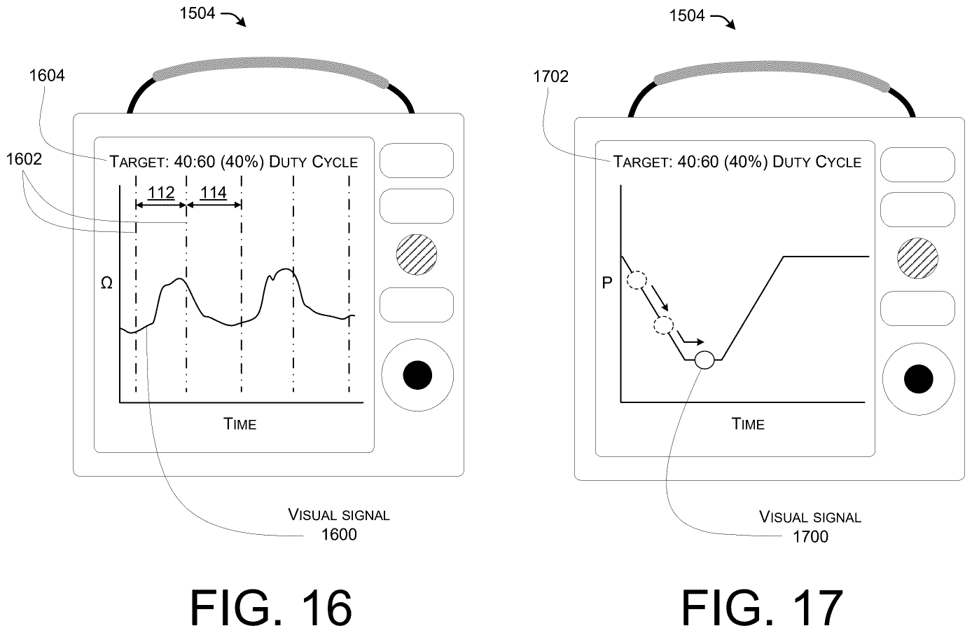

In some examples, the defibrillator 1504 instructs the rescuer 204 to apply electrode pads 1506 (which may be the same as, or similar to, the electrode pads 210 introduced in FIG. 2) to the skin of the subject 104. For example, the electrode pads 1506 may be attached to a thorax of the subject 104 or otherwise adhered to skin on the thorax (e.g., the chest) of the subject 104. In some examples, the processor(s) 1312 of the defibrillator 1504 generates an impedance signal using the electrode pads 1506, such as the impedance signal described above with respect to FIG. 13. The impedance signal can be indicative of thoracic impedance (or transthoracic impedance) of the subject 104. As described in more detail below with respect to FIG. 16, the impedance signal can include chest compression artifacts. That is, the chest compressions that the rescuer 204 is manually administering to the subject 104 can induce changes in the impedance signal, and such compression-induced changes in the impedance signal can be analyzed to determine an administered duty cycle of the chest compressions that the rescuer 204 manually administered, or is manually administering, to the subject 104. More generally, the processor(s) 1312 of the defibrillator 1504 can analyze compression-induced changes in the impedance signal to determine the quality of manual CPR that was, or is being, administered to the subject 104.

In some examples, the CPR duty cycle guidance system 1502 includes a rescuer device 1508, which may be the same as, or similar to, the rescuer device 212 introduced in FIG. 2. In some examples, the CPR duty cycle guidance system 1502 includes a wearable device 1510, such as a smart watch, a HMD, or any other suitable wearable device 1510 configured to be worn by the rescuer 204. In some examples, the CPR duty cycle guidance system 1502 includes the defibrillator 1504 exclusively, the rescuer device 1508 exclusively, the wearable device 1510 exclusively, or any combination of one or more of the defibrillator 1504, the rescuer device 1508, and the wearable device 1510. In some examples, the rescuer device 1508 and/or the wearable device 1510 are configured to be wirelessly paired or coupled via one or cables or wires to each other and/or to the defibrillator 1504. Moreover, it is to be appreciated that a CPR duty cycle guidance system (e.g., the system 202 of FIG. 2) may include a CPR feedback device 206, as described above in more detail.

In some examples, the CPR duty cycle guidance system 1502 is configured to guide (or coach) the rescuer 204 to administer manual chest compressions at or near a target duty cycle (e.g., a 40:60 duty cycle). In some examples, the CPR duty cycle guidance system 1502 evaluates the manual CPR being administered and guides (or coaches) the rescuer to adjust, correct, and/or augment their administered duty cycle. In some examples, the CPR duty cycle guidance system 1502 is configured to provide guidance without evaluating the manual CPR being administered. That is, the CPR duty cycle guidance system 1502 may blindly output guidance for the rescuer 204 without basing the guidance on the chest compressions that are being administered manually by the rescuer 204. In some examples, the defibrillator 1504 is configured to output a signal(s) as guidance for the rescuer 204 and/or as feedback for the rescuer 204 to dynamically adjust a duty cycle of the chest compressions. In some examples, the rescuer device 1508 and/or the wearable device 1510 (and/or the CPR feedback device 206 of FIG. 2) is/are configured to output a signal(s) as guidance for the rescuer 204 and/or as feedback for the rescuer 204 to dynamically adjust a duty cycle of the chest compressions. The signal(s) that is output as guidance for the rescuer 204 and/or as feedback for the rescuer 204 may include an audible signal(s), a visual signal(s), and/or a haptic signal(s). The CPR duty cycle guidance system 1502 may be used as a training aide and/or for real-word cardiac arrest deployment.

FIG. 15 illustrates an example visual signal(s) 1512 that may be output by the defibrillator 1504 as guidance for the rescuer 204 and/or as feedback for the rescuer 204 to dynamically adjust a duty cycle of the chest compressions. The visual signal(s) 1512 may be output via a display(s) of the defibrillator 1504, one or more light emitting elements (e.g., a LED(s) of the defibrillator 1504, and/or other output devices of the defibrillator 1504. In some examples, the rescuer device 1508 and/or the wearable device 1510 (and/or the CPR feedback device 206 of FIG. 2) is configured to output a similar visual signal(s) 1512 via an output device(s) (e.g., a display(s)) thereof. In the example of FIG. 15, the visual signal(s) 1512 includes a moving icon that tracks along a waveform exhibiting the target duty cycle (e.g., a 40:60 duty cycle), with time on the x-axis and position (e.g., of a point on the surface of the subject's chest) on the y-axis. This allows the rescuer 204 to visualize, in real-time, the current time point during a compression-decompression cycle so that the rescuer 204 can administer chest compressions at, or as close as possible to, the target duty cycle. This example and other examples of the visual signal(s) 1512 are described in more detail below with respect to FIGS. 16, 17, 18, 19A, 19B, 19C, 20, 21A, and 21B.

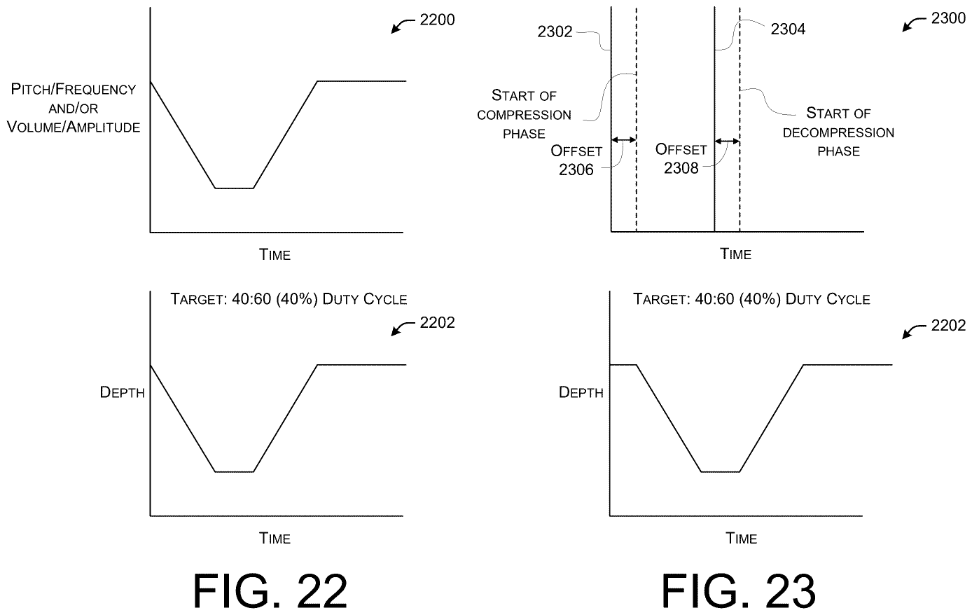

FIG. 15 further illustrates an example audible signal(s) 1514 that may be output by the defibrillator 1504 as guidance for the rescuer 204 and/or as feedback for the rescuer 204 to dynamically adjust a duty cycle of the chest compressions. The audible signal(s) 1514 may be output via a speaker(s) of the defibrillator 1504. In some examples, the rescuer device 1508 and/or the wearable device 1510 (and/or the CPR feedback device 206 of FIG. 2) is/are configured to output a similar audible signal(s) 1514 via a speaker(s) thereof. In some examples, the audible signal(s) 1514 includes a sustained sound that changes pitch and/or volume in accordance with a waveform exhibiting the target duty cycle (e.g., a 40:60 duty cycle), a repeating sound (e.g., tone, beat, etc.) that demarcates the compression phase and the decompression phase of individual compression-decompression cycles and between sequential compression-decompression cycles, and/or other types of audible signals. These examples and other examples of the audible signal(s) 1514 are described in more detail below with respect to FIGS. 22 and 23.

FIG. 15 further illustrates an example haptic signal(s) 1516 that may be output by the wearable device 1510 (and/or the CPR feedback device 206 of FIG. 2) as guidance for the rescuer 204 and/or as feedback for the rescuer 204 to dynamically adjust a duty cycle of the chest compressions. The haptic signal(s) 1516 may be output via a haptic actuator(s) (e.g., a LRA, an ERM, a piezoelectric actuator, etc.) of the wearable device 1510 (and/or the CPR feedback device 206 of FIG. 2). In some examples, the defibrillator 1504 and/or the rescuer device 1508 is configured to cause the haptic signal(s) 1516 to be output by a device with the haptic actuator(s), such as by transmitting an instruction(s) to output the haptic signal(s) 1516. In some examples, the haptic signal(s) 1516 includes a sustained vibration that changes frequency and/or amplitude in accordance with a waveform exhibiting the target duty cycle (e.g., a 40:60 duty cycle), a repeating vibration (e.g., buzz, tick, etc.) that demarcates the compression phase and the decompression phase of individual compression-decompression cycles and between sequential compression-decompression cycles, and/or other types of haptic signals. These examples and other examples of the haptic signal(s) 1516 are described in more detail below with respect to FIGS. 22 and 23.

Upon receiving the guidance and/or the feedback from the CPR duty cycle guidance system 1502, the rescuer 204 can utilize the guidance and/or the feedback to ensure that their technique for administering the chest compressions to the subject 104 is in alignment with the target duty cycle (e.g., a 40:60 duty cycle) and/or to adjust their technique, if necessary. For example, by hearing a sound(s) that is being output audibly, by feeling a vibration(s) that is being output haptically, and/or by seeing a visual signal(s) (e.g., a displayed visual signal(s), a light(s), etc.) that is being output visually, by the CPR duty cycle guidance system 1502 of FIG. 15 (or the system 202 of FIG. 2), the rescuer 204 can manually administer chest compressions in accordance with the target duty cycle and/or adjust their technique, if necessary. For example, if the target duty cycle is a 40:60 duty cycle, the rescuer 204 may be guided (or coached) by the CPR duty cycle guidance system 1502 to compress the chest of the subject 104 to a target compression depth, pause at the target compression depth, and release the compression for a longer period of time than the period of time that the rescuer 204 compresses and holds the chest in the compressed state. In other words, the guidance and/or the feedback provided to the rescuer 204 may be specifically designed for the rescuer 204 to administer chest compressions in accordance with a target duty cycle.

Although the CPR duty cycle guidance system 1502 (or the system 202 of FIG. 2) may exclusively output a visual signal(s) 1512, an audible signal(s) 1514, or a haptic signal(s) 1516 as guidance and/or feedback for the rescuer 204 to administer chest compressions at or near a target duty cycle, there may be benefits to outputting a combination of two or more signal types for the rescuer 204, such as outputting a visual signal(s) 1512 and an audible signal(s) 1514, outputting a visual signal(s) 1512 and a haptic signal(s) 1516, outputting an audible signal(s) 1514 and a haptic signal(s) 1516, or outputting a visual signal(s) 1512, an audible signal(s) 1514, and a haptic signal(s) 1516. For example, the audible signal(s) 1514 and/or the haptic signal(s) 1516 may be output individually or together simultaneously to provide real-time duty cycle guidance from one compression-decompression cycle to the next, and the visual signal(s) 1512 may be output in combination with the audible signal(s) 1514 and/or the haptic signal(s) 1516 to provide guidance at a coarser, less frequent level, such as by outputting a visual signal(s) 1512 that indicates whether the rescuer 204 is doing well or should adjust their technique. In this example, the visual signal(s) 1512 may instruct the rescuer 204 to shorten the compression phase or lengthen the compression phase, as the case may be, which the audio signal(s) 1514 and/or the haptic signal(s) 1516 is providing cycle-to-cycle guidance to the rescuer 204. In other words, the visual signal(s) 1512 can be output over a span of multiple compression-decompression cycles, which, in combination with cycle-to-cycle guidance via an audible signal(s) 1514 and/or a haptic signal(s) 1516, may provide a holistic approach to guiding the rescuer 204 for administering chest compressions at or near the target duty cycle. In some examples, the visual signal(s) 1512 may instruct the rescuer 204 to listen to the audible signal(s) 1514 and/or to feel the haptic signal(s) 1516, and/or the audible signal(s) 1514 may instruct the rescuer 204 to turn their gaze toward the visual signal(s) 1512. It is to be appreciated that verbal guidance and/or instructions can be provided to the rescuer 204 via the visual signal(s) 1512 and/or the audible signal(s) 1514, in some examples, such as verbal guidance on how to administer chest compressions, a qualitative assessment of the rescuer's CPR technique in verbal form, and/or verbal instructions as to how to adjust the duty cycle of chest compressions.

In some examples, different time aspects (e.g., past, present, and future) can be utilized in the different types of signals; namely, the visual signal(s) 1512, the audible signal(s) 1514, and/or the haptic signal(s) 1516. For example, the visual signal(s) 1512 may provide the rescuer 204 with guidance regarding the current (present) duty cycle of chest compressions, and that audible signal(s) 1514 and/or the haptic signal(s) 1516 may provide the rescuer 204 with guidance regarding previous (past) duty cycles of chest compressions to estimate the administered duty cycle and provide guidance for the upcoming (future) duty cycle of chest compression. The combination of these different time aspects may be more beneficial than providing guidance exclusively via a single type of signal; namely, the visual signal(s) 1512, the audible signal(s) 1514, or the haptic signal(s) 1516.

FIG. 16 illustrates an example visual signal 1600 presented on a display to provide duty cycle guidance to a rescuer 204 who is manually administering chest compressions to the subject 104. The visual signal 1600 is an example of the visual signal(s) 1512 described above with reference to FIG. 15. In the example of FIG. 16, the visual signal 1600 is presented on a display of the defibrillator 1504, but it is to be appreciated that the visual signal 1600 may be presented on other display devices of the CPR duty cycle guidance system 1502 (or the system 202 of FIG. 2). The visual signal 1600 represents an impedance signal generated using the electrode pads 1506 of the defibrillator 1504, the electrode pads 1506 being configured to attach to a thorax of the subject 104. In some examples, the processor(s) 1312 of the defibrillator 1504 generates the visual signal 1600 (e.g., impedance signal), and causes the display of the defibrillator 1504 to present the visual signal 1600 (e.g., impedance signal), with time (e.g., in seconds) on the x-axis and impedance (e.g., in ohms) on the y-axis.

As mentioned above, the impedance signal can include chest compression artifacts, which typically present as repeating Gaussian-like curves. That is, during an individual compression-decompression cycle, the impedance may increase as the rescuer 204 compresses the chest of the subject 104 during the compression phase 112, and the impedance may decrease as the rescuer 204 releases the compression during the decompression phase 114. Whether impedance changes are directly proportional or inversely proportional to the positional changes of the chest during CPR may depend on the placement of the electrode pads 1506 (e.g., anterior-posterior (AP) pad placement, anterior-lateral (AL) pad placement, etc.). Compression-induced changes in the impedance signal can be analyzed to determine an administered duty cycle of the chest compressions that the rescuer 204 manually administered, or is manually administering, to the subject 104. In this sense, the visual signal 1600 (e.g., impedance signal) is indicative of an administered duty cycle(s) of the chest compressions that the rescuer 204 manually administered, or is manually administering, to the subject 104. Accordingly, the visual signal 1600 (e.g., impedance signal) can be presented as guidance for the rescuer 204 who is manually administering the chest compression to the subject 104 because the rescuer 204 can see, from the visual signal 1600 (e.g., impedance signal), the duty cycle of the chest compressions they are administering to the subject 104 as a form of feedback for the rescuer 204 to ensure that they are administering chest compressions in accordance with a target duty cycle. The visual signal 1600 (e.g., impedance signal) may be readily understood by a trained rescuer 204 who has been trained to administer CPR and understands that chest compressions artifacts are exhibited in the impedance signal.

In some examples, the visual signal 1600 includes vertically-oriented lines 1602 that demarcate the compression phase 112 and the decompression phase 114 of individual compression-decompression cycles. For example, the processor(s) 1312 of the defibrillator 1504 may be configured to analyze the impedance signal to identify peaks and/or valleys in the impedance signal as likely transition points between the compression phase 112 and the decompression phase 114, and the processor(s) 1312 may be configured to generate, and cause display of, vertically-oriented lines 1602 that indicate these transition points. The vertically-oriented lines 1602 can make it easier for the rescuer 204 to discern the boundary between the compression phase 112 and the decompression phase 114 for an individual compression-decompression cycle and/or the boundary between sequential compression-decompression cycles. In some examples, the visual signal 1600 includes text 1604 that represents the duty cycle ratio (or percentage), such as 40:60 (or 40%), to clearly indicate the target duty cycle to the rescuer 204.

FIG. 17 illustrates an example visual signal 1700 presented on a display to provide duty cycle guidance to a rescuer 204 who is manually administering chest compressions to the subject 104. The visual signal 1700 is an example of the visual signal(s) 1512 described above with reference to FIG. 15. In the example of FIG. 17, the visual signal 1700 is presented on a display of the defibrillator 1504, but it is to be appreciated that the visual signal 1700 may be presented on other display devices of the CPR duty cycle guidance system 1502 (or the system 202 of FIG. 2). The visual signal 1700 is presented as a moving icon that tracks along a waveform exhibiting the target duty cycle), with time (e.g., in seconds) on the x-axis and position (e.g., in centimeters) on the y-axis. In this sense, the visual signal 1700 is indicative of a target duty cycle of chest compressions (e.g., a 40:60 duty cycle). In the example of FIG. 17, the waveform exhibiting the target duty cycle spans a single compression-decompression cycle, and the icon moves across the display screen in a rightward direction as the icon tracks along the waveform. The icon also moves downward and upward on the display screen over time as the target position (e.g., a position of a point on the surface of the subject's chest) changes due to the compression or decompression of the chest of the subject 104. As mentioned above, this allows the rescuer 204 to visualize, in real-time, the current time point during a compression-decompression cycle so that the rescuer 204 can administer chest compressions at, or as close as possible to, the target duty cycle. Although the moving icon is depicted as a circular icon in FIG. 17, it is to be appreciated that the moving icon can be presented as other shapes, graphics, symbols, and/or visual indicia. Furthermore, although the icon is shown as moving in the rightward direction across the display screen in FIG. 17, the icon can move in other directions, such as in a downward direction, an upward direction, a leftward direction, or diagonally. The rightward direction in which the icon is shown as moving across the display screen is intuitive for the rescuer 204 as indicating the passage of time. In some examples, the visual signal 1700 includes text 1702 that represents the duty cycle ratio (or percentage), such as 40:60 (or 40%), to clearly indicate the target duty cycle to the rescuer 204.

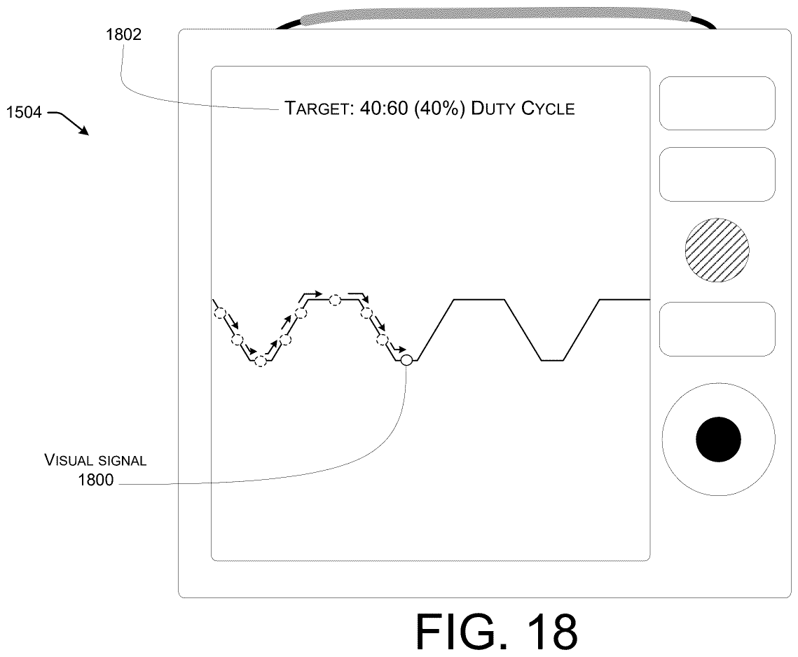

FIG. 18 illustrates an example visual signal 1800 presented on a display to provide duty cycle guidance to a rescuer 204 who is manually administering chest compressions to the subject 104. The visual signal 1800 is an example of the visual signal(s) 1512 described above with reference to FIG. 15. In the example of FIG. 18, the visual signal 1800 is presented on a display of the defibrillator 1504, but it is to be appreciated that the visual signal 1800 may be presented on other display devices of the CPR duty cycle guidance system 1502 (or the system 202 of FIG. 2). The visual signal 1800 is presented as a moving icon that tracks along a waveform exhibiting the target duty cycle), with time (e.g., in seconds) on the x-axis and position (e.g., in centimeters) on the y-axis. In this sense, the visual signal 1800 is indicative of a target duty cycle of chest compressions (e.g., a 40:60 duty cycle). In the example of FIG. 18, the waveform exhibiting the target duty cycle spans multiple (e.g., three) compression-decompression cycles to provide a wider field-of-view of the target duty cycle, as compared to the waveform depicted in FIG. 17, for example, which spans a single compression-decompression cycle. Although the example of FIG. 18 depicts a waveform that spans three compression-decompression cycles, it is to be appreciated that the visual signal 1800 may be presented as a moving icon that tracks along a waveform spanning N compression-decompression cycles, where N is a number greater than one (e.g., N=8). This wider field-of-view can make it easier for the rescuer 204 to track the moving icon as the icon moves in the rightward direction across the display screen, as compared to the moving icon resetting itself at the left side of the display screen at the start of every compression-decompression cycle, as is the case with the visual signal 1700 of FIG. 17.

Accordingly, in the example of FIG. 18, the moving icon continues moving in a rightward direction across the display as the moving icon transitions from a first compression-decompression cycle to a second compression-decompression cycle to a third compression-decompression cycle, and so on and so forth, until there is no more room on the display screen for the icon to continue moving in the rightward direction, at which point the icon resets itself at the left side of the display and iterates the rightward movement as the icon tracks along the waveform spanning multiple compression-decompression cycles. This allows the rescuer 204 to visualize, in real-time, the current time point during a compression-decompression cycle over the timespan of several seconds so that the rescuer 204 can administer chest compressions at, or as close as possible to, the target duty cycle. Again, although the moving icon is depicted as a circular icon in FIG. 18, it is to be appreciated that the moving icon can be presented as other shapes, graphics, symbols, and/or visual indicia. Furthermore, although the icon is shown as moving in the rightward direction across the display screen in FIG. 18, the icon can move in other directions, such as in a downward direction, an upward direction, a leftward direction, or diagonally. In some examples, the visual signal 1800 includes text 1802 that represents the duty cycle ratio (or percentage), such as 40:60 (or 40%), to clearly indicate the target duty cycle to the rescuer 204.

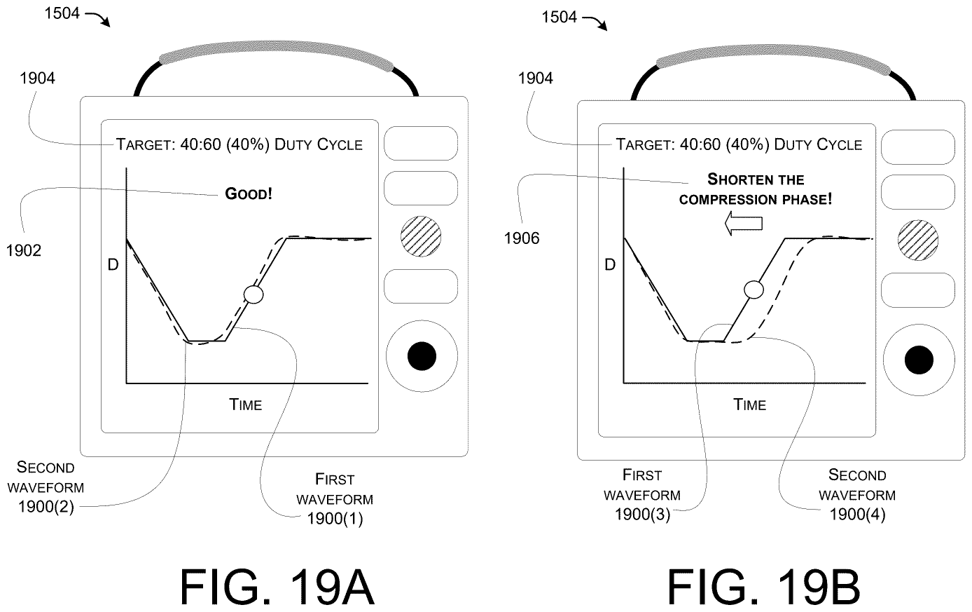

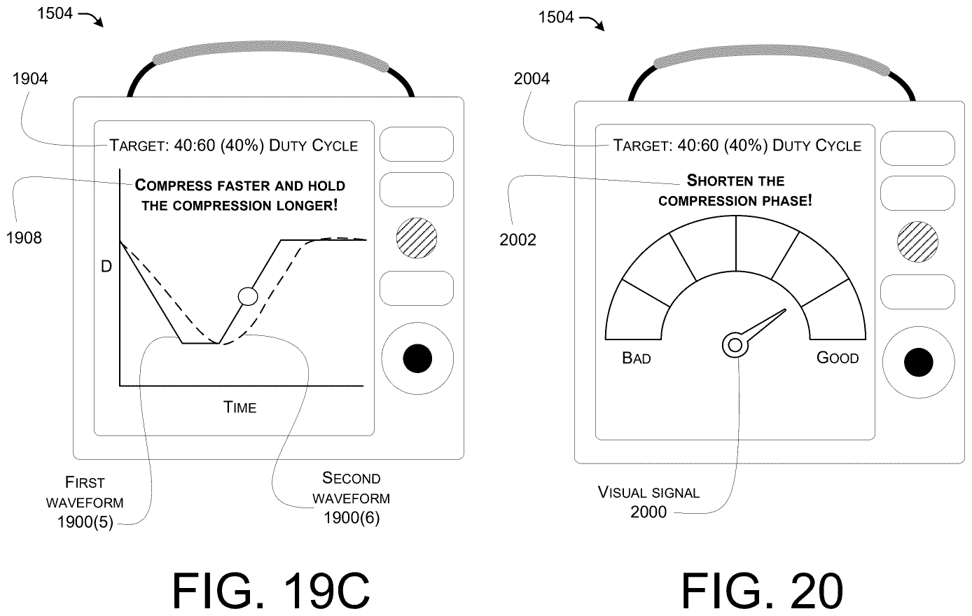

FIGS. 19A-19C illustrate example waveforms presented on a display to provide duty cycle guidance to a rescuer 204 who is manually administering chest compressions to the subject 104. In the example of FIG. 19A, the processor(s) 1312 of the defibrillator 1504 causes a first waveform 1900(1) and a second waveform 1900(2) to be presented on the display. The waveforms 1900 are examples of the visual signal(s) 1512 described above with reference to FIG. 15. In the example of FIGS. 19A-19C, the waveforms 1900 are presented on a display of the defibrillator 1504, but it is to be appreciated that the waveforms 1900 may be presented on other display devices of the CPR duty cycle guidance system 1502 (or the system 202 of FIG. 2).

The first waveform 1900(1) exhibits a target duty cycle (e.g., a 40:60 duty cycle). In some examples, the first waveform 1900(1) is presented with a moving icon that tracks along the waveform as guidance for the rescuer 204, as described above with reference to FIGS. 17 and 18. The second waveform 1900(2) exhibits an administered duty cycle of the chest compressions that the rescuer 204 manually administered, or is manually administering, to the subject 104. By presenting both waveforms 1900(1) and 1900(2) (e.g., simultaneously), the rescuer 204 can compare the waveforms 1900 to understand whether they are administering chest compressions in accordance with the target duty cycle or not. In the example of FIG. 19A, the second waveform 1900(2) is substantially aligned with the first waveform 1900(1), which indicates that the rescuer 204 is administering chest compressions correctly (at least in terms of adhering to the target duty cycle).

As shown in FIG. 19A, the second waveform 1900(2) is overlaid on top of the first waveform 1900(1) and is aligned with the first waveform 1900(1) on a time axis (or x-axis). The second waveform 1900(2) is also aligned with the first waveform 1900(1) on a position axis (or y-axis) in FIG. 19A. This allows the rescuer 204 to see how well the second waveform 1900(2) “fits” the first waveform 1900(1). In some examples, both waveforms 1900(1) and 1900(2) are presented on the display contemporaneously and aligned on a time axis, but with one waveform 1900 above or below the other waveform 1900. For example, the first waveform 1900(1) can be presented above the second waveform 1900(2), or vice versa. In some examples, the first waveform 1900(1) is presented to the right or to the left of the second waveform 1900(2).

In some examples, the processor(s) 1312 of the defibrillator 1504 is configured to determine an administered duty cycle of the chest compressions that the rescuer manually administered, or is manually administering, to the subject 104 in order to generate and display the second waveform 1900(2) exhibiting the administered duty cycle. In some examples, the processor(s) 1312 determines the administered duty cycle based on (e.g., by analyzing) the impedance signal generated using the electrode pads 1506 of the defibrillator 1504. For example, the processor(s) 1312 may identify peaks and valleys in the impedance signal to determine time points that correspond to transitions between the compression phase 112 and the decompression phase 114 of individual compression-decompression cycles, and the processor(s) 1312 may plot position (e.g., a position of a point on a surface of a chest of the subject 104) over time based on the amount of change in the impedance over time. In some examples, the processor(s) 1312 determines the administered duty cycle based on (e.g., by analyzing) a compression parameter(s) sensed by a sensor(s) configured to be disposed on, or in proximity to, a chest of the subject 104, such as the sensor(s) 214 of the CPR feedback device 206 described above with reference to FIG. 2.

In some examples, the processor(s) 1312 may compare the administered duty cycle to the target duty cycle and may cause the display to present a visual signal(s) as feedback for the rescuer 204. For example, the processor(s) 1312 may cause the display to present text 1902 that indicates, to the rescuer 204, that they are administering chest compressions correctly in accordance with the target duty cycle (e.g., the text “Good!”). In some examples, the processor(s) 1312 causes the display to highlight the text 1902, the first waveform 1900(1), and/or the second waveform 1900(2) in a color as guidance and/or as feedback for the rescuer 204. In the example of FIG. 19A, the text 1902 may be highlighted in the color green to indicate the rescuer 204 is substantially “hitting” the target duty cycle. Additionally, or alternatively, the first waveform 1900(1), or a portion thereof, may be highlighted in the color green, and/or the second waveform 1900(2), or a portion thereof, may be highlighted in the color green to indicate that the rescuer 204 is substantially hitting the target duty cycle. In some examples, the waveforms displayed in the visual signals 1700 and 1800 of FIGS. 17 and 18, respectively, may be highlighted in a color (e.g., the color green) to indicate that the rescuer 204 is substantially hitting the target duty cycle. In some examples, the display in FIG. 19A presents text 1904 that represents the duty cycle ratio (or percentage), such as 40:60 (or 40%), to clearly indicate the target duty cycle to the rescuer 204.

In the example of FIG. 19B, the first waveform 1900(3) exhibits a target duty cycle (e.g., a 40:60 duty cycle). In some examples, the first waveform 1900(3) is presented with a moving icon that tracks along the waveform as guidance for the rescuer 204, as described above with reference to FIGS. 17 and 18. The second waveform 1900(4) exhibits an administered duty cycle of the chest compressions that the rescuer 204 manually administered, or is manually administering, to the subject 104. In the example of FIG. 19B, the second waveform 1900(4) is not aligned with the first waveform 1900(3), which indicates that the rescuer 204 is administering chest compressions incorrectly (at least in terms of failing to adhere closely enough to the target duty cycle). In some examples, the processor(s) 1312 may compare the administered duty cycle to the target duty cycle and may cause the display to present a visual signal(s) as feedback for the rescuer 204. For example, the processor(s) 1312 may cause the display to present a visual signal(s) 1906 that provides guidance to the rescuer 204 to adjust the administered duty cycle (e.g., the text “Shorten the compression phase!” with an arrow pointing to the left to indicate that the compression phase 112 should be shortened to hit the target duty cycle). Although the visual signal(s) 1906 depicts an arrow pointing to the left to indicate that the compression phase 112 should be shortened, it is to be appreciated that the visual signal(s) 1906 may present an arrow pointing to the right when the compression phase 112 should be lengthened, and/or the direction of the arrow may indicate a recommended shortening or lengthening of the decompression phase 114, rather than the compression phase 112. It is also to be appreciated that the arrow may point upward or downward to indicate similar feedback to the rescuer 204, where an upward-pointing arrow can indicate that the compression phase 112 (or the decompression phase 114) should be lengthened, and a downward-pointing arrow can indicate that the compression phase 112 (or the decompression phase 114) should be shortened.

In some examples, the processor(s) 1312 causes the display to highlight the visual signal(s) 1906, the first waveform 1900(3), and/or the second waveform 1900(4) in a color as guidance and/or feedback for the rescuer 204. In the example of FIG. 19B, the visual signal(s) 1906 may be highlighted in the color red to provide guidance to the rescuer 204 to adjust the administered duty cycle. Additionally, or alternatively, the first waveform 1900(3), or a portion thereof, may be highlighted in the color red, and/or the second waveform 1900(4), or a portion thereof, may be highlighted in the color red to provide guidance to the rescuer 204 to adjust the administered duty cycle. In some examples, the waveforms displayed in the visual signals 1700 and 1800 of FIGS. 17 and 18, respectively, may be highlighted in the color red to provide guidance to the rescuer 204 to adjust the administered duty cycle. In some examples, the display in FIG. 19B presents the text 1904 that represents the duty cycle ratio (or percentage), such as 40:60 (or 40%), to clearly indicate the target duty cycle to the rescuer 204.

In the example of FIG. 19C, the first waveform 1900(5) exhibits a target duty cycle (e.g., a 40:60 duty cycle). In some examples, the first waveform 1900(5) is presented with a moving icon that tracks along the waveform as guidance for the rescuer 204, as described above with reference to FIGS. 17 and 18. The second waveform 1900(6) exhibits an administered duty cycle of the chest compressions that the rescuer 204 manually administered, or is manually administering, to the subject 104. In the example of FIG. 19C, the second waveform 1900(6) is not aligned with the first waveform 1900(5), which indicates that the rescuer 204 is administering chest compressions incorrectly (at least in terms of failing to adhere closely enough to the target duty cycle). In some examples, the processor(s) 1312 may compare the administered duty cycle to the target duty cycle and may cause the display to present a visual signal(s) as feedback for the rescuer 204. For example, the processor(s) 1312 may cause the display to present text 1908 that provides guidance to the rescuer 204 to adjust the administered duty cycle (e.g., the text “Compress faster and hold the compression longer!”). This guidance can be based on a determination, from comparing the administered duty cycle to the target duty cycle, that the rescuer 204 is not keeping a chest of the subject 104 in a compressed state (and/or in a decompressed state) for a threshold period of time. As discussed above with respect to FIG. 3, for a target duty cycle having a compression phase 112 that is shorter than the decompression phase 114, such as the example 40:60 duty cycle, the first hold period 302(1) (e.g., plateau period) in the waveform 300 allows the aortic valve to close before the compression phase 112 ends, and the second hold period 302(2) (e.g., another plateau period) in the waveform 300 allows the heart to fill with blood in a natural and undisturbed manner. Accordingly, the processor(s) 1312 may be configured to determine whether the second waveform 1900(6) includes an above-threshold hold period(s), like the hold periods exhibited in the first waveform 1900(5).

In some examples, the processor(s) 1312 causes the display to highlight the text 1908, the first waveform 1900(5), and/or the second waveform 1900(6) in a color as guidance and/or feedback for the rescuer 204. In the example of FIG. 19C, the text 1908 may be highlighted in the color red to provide guidance to the rescuer 204 to adjust the administered duty cycle. Additionally, or alternatively, the first waveform 1900(5), or a portion thereof, may be highlighted in the color red, and/or the second waveform 1900(6), or a portion thereof, may be highlighted in the color red to provide guidance to the rescuer 204 to adjust the administered duty cycle. In some examples, the display in FIG. 19C presents the text 1904 that represents the duty cycle ratio (or percentage), such as 40:60 (or 40%), to clearly indicate the target duty cycle to the rescuer 204. In some examples, the first and second waveforms 1900 that are presented on the display contemporaneously in FIGS. 19A-19C can be presented in different colors, respectively, and/or as solid and dashed lines, respectively. These visual aids can help the rescuer 204 differentiate between the two waveforms 1900.

FIG. 20 illustrates an example visual signal 2000 presented on a display to provide duty cycle guidance to a rescuer 204 who is manually administering chest compressions to the subject 104. The visual signal 2000 is an example of the visual signal(s) 1512 described above with reference to FIG. 15. In the example of FIG. 20, the visual signal 2000 is presented on a display of the defibrillator 1504, but it is to be appreciated that the visual signal 2000 may be presented on other display devices of the CPR duty cycle guidance system 1502 (or the system 202 of FIG. 2). The visual signal 2000 is presented as a gauge with a moving needle to guide the rescuer 204 to adjust the administered duty cycle or to refrain from adjusting the administered duty cycle. For example, depending on where the needle is pointing, the visual signal 2000 provides guidance and/or feedback to the rescuer 204 so that the rescuer 204 can understand whether they are administering chest compressions in accordance with the target duty cycle or not. In the example of FIG. 20, the needle is pointing more towards the “Good” end of the gauge than the “Bad” end of the gauge, which indicates that the rescuer 204 is administering chest compressions substantially correctly (at least in terms of adhering to the target duty cycle). However, because the needle is not pointing directly at the “Good” end, the visual signal 200 indicates that there is some room for slight improvement of the administered chest compressions to adhere more closely to the target duty cycle.

In some examples, the processor(s) 1312 may compare the administered duty cycle to the target duty cycle and may cause the display to present the visual signal(s) 2000 as feedback for the rescuer 204. In some examples, the visual signal 2000 may further include, and/or the processor(s) 1312 may cause the display to present, text 2002 that provides additional guidance to the rescuer 204 to adjust the administered duty cycle (e.g., the text “Shorten the compression phase!”). This guidance can be based on the processor(s) 1312 determining, by comparing the administered duty cycle to the target duty cycle, that the compression phase 112 of the administered duty cycle is too long (e.g., the administered duty cycle might be a 50:50 duty cycle, and the target duty cycle might be a 40:60 duty cycle). In some examples, the processor(s) 1312 determines the administered duty cycle based on (e.g., by analyzing) the impedance signal generated using the electrode pads 1506 of the defibrillator 1504, as described above. In some examples, the processor(s) 1312 determines the administered duty cycle based on (e.g., by analyzing) a compression parameter(s) sensed by a sensor(s) configured to be disposed on, or in proximity to, a chest of the subject 104, such as the sensor(s) 214 of the CPR feedback device 206 described above with reference to FIG. 2. In some examples, the processor(s) 1312 causes the display to highlight the text 2002 and/or the visual signal 2000 in a color as guidance and/or as feedback for the rescuer 204. In the example of FIG. 20, the text 2002 and/or the visual signal 2000 may be highlighted in the color orange to indicate the rescuer 204 is close to, but not quite hitting, the target duty cycle. In some examples, the display in FIG. 20 presents text 2004 that represents the duty cycle ratio (or percentage), such as 40:60 (or 40%), to clearly indicate the target duty cycle to the rescuer 204.

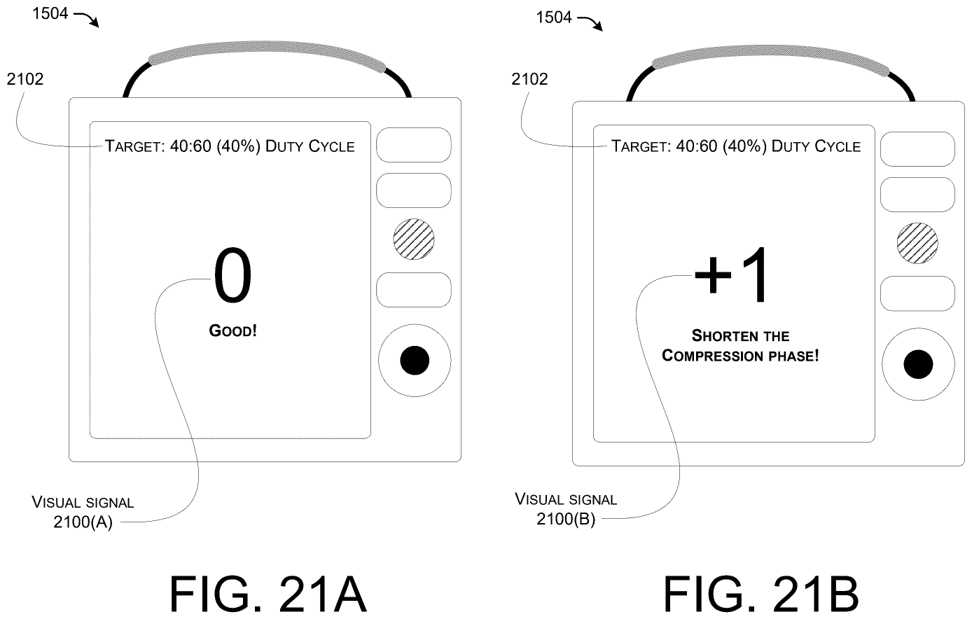

FIGS. 21A and 21B illustrate an example visual signal 2100 presented on a display to provide duty cycle guidance to a rescuer 204 who is manually administering chest compressions to the subject 104. The visual signal 2100 is an example of the visual signal(s) 1512 described above with reference to FIG. 15. In the example of FIGS. 21A and 21B, the visual signal 2100 is presented on a display of the defibrillator 1504, but it is to be appreciated that the visual signal 2100 may be presented on other display devices of the CPR duty cycle guidance system 1502 (or the system 202 of FIG. 2). The visual signal 2100 can be presented as a positive numerical value, a negative numerical value, or a numerical value of zero to guide the rescuer 204 to adjust the administered duty cycle or to refrain from adjusting the administered duty cycle. For example, in FIG. 21A, the visual signal 2100(A) is presented as a numerical value of zero, which indicates, to the rescuer 204, that they are administering chest compressions in accordance with the target duty cycle. In some examples, the visual signal 2100(A) further includes text (e.g., the text “Good!”) to indicate that the rescuer 204 is administering chest compressions correctly in accordance with the target duty cycle. In

FIG. 21B, the visual signal 2100(B) is presented as a positive numerical value (e.g., a numerical value of +1), which provides guidance to the rescuer 204 to adjust the administered duty cycle in a particular direction (e.g., to shorten the compression phase 112). In some examples, the visual signal 2100(B) further includes text (e.g., the text “Shorten the compression phase!”) to indicate that the compression phase 112 should be shortened to more closely match the target duty cycle. If the visual signal 2100 were to be presented as a negative numerical value (e.g., a numerical value of −1), this can provide guidance to the rescuer 204 to adjust the administered duty cycle in the opposite direction (e.g., to lengthen the compression phase 112). It is also to be appreciated that the visual signal 2100 may provide guidance to the rescuer 204 to adjust the decompression phase 114 in one direction or the other (e.g., to shorten or lengthen the decompression phase 114), rather than the compression phase 112. In some examples, the range of numerical values that can be presented in the visual signal 2100 is bounded or capped at minimum and maximum numerical values. For example, the numerical values that are presentable may range from negative three to positive three, or any other suitable value range. A higher absolute numerical value indicates to the rescuer 204 that a larger scale adjustment to the administered duty cycle is recommended, whereas a lower absolute numerical value indicates to the rescuer 204 that a smaller scale adjustment to the administered duty cycle is recommended.

In some examples, the processor(s) 1312 causes the display to highlight the visual signal(s) 2100 in a color as guidance and/or feedback for the rescuer 204. In the example of FIG. 21A, the visual signal(s) 2100(A) may be highlighted in the color green to indicate the rescuer 204 is substantially hitting the target duty cycle. In the example of FIG. 21B, the visual signal(s) 2100(B) may be highlighted in the color red to provide guidance to the rescuer 204 to adjust the administered duty cycle. In some examples, the processor(s) 1312 may compare the administered duty cycle to the target duty cycle and may cause the display to present the visual signal(s) 2100 as feedback for the rescuer 204. In some examples, the processor(s) 1312 determines the administered duty cycle based on (e.g., by analyzing) the impedance signal generated using the electrode pads 1506 of the defibrillator 1504, as described above. In some examples, the processor(s) 1312 determines the administered duty cycle based on (e.g., by analyzing) a compression parameter(s) sensed by a sensor(s) configured to be disposed on, or in proximity to, a chest of the subject 104, such as the sensor(s) 214 of the CPR feedback device 206 described above with reference to FIG. 2. In some examples, the display in FIGS. 21A and 21B presents text 2102 that represents the duty cycle ratio (or percentage), such as 40:60 (or 40%), to clearly indicate the target duty cycle to the rescuer 204.

In some examples, the visual signal(s) 1512 described herein can provide gamification aspects in duty cycle guidance, which can increase engagement of the rescuer 204 with the duty cycle guidance. For example, as the rescuer 204 is administering chest compressions to the subject 104, the rescuer 204 may be incentivized to try to cause the visual signal(s) 1512 to be highlighted in the color green, and/or to move the needle in FIG. 20 to the “Good” end of the gauge, and/or to maintain the numerical value at zero in the example of FIGS. 21A and 21B.

FIG. 22 illustrates an example signal 2200 (audible and/or haptic) that may be output (via a speaker(s) and/or a haptic actuator(s)) to provide duty cycle guidance to a rescuer 204 who is manually administering chest compressions to the subject 104. The signal 2200 is an example of the audible signal(s) 1514 and/or the haptic signal(s) 1516 described above with reference to FIG. 15. The signal 2200 can be output via a speaker(s) and/or a haptic actuator(s) of any device of the CPR duty cycle guidance system 1502 (or the system 202 of FIG. 2) that includes a speaker(s) and/or a haptic actuator(s). For example, the signal 2200 can be output as an audible signal 1514 via a speaker(s) of the defibrillator 1504 and/or the signal 2200 can be output as a haptic signal 1516 via a haptic actuator(s) of the CPR feedback device 206 of FIG. 2 and/or a haptic actuator(s) of the wearable device 1510 of FIG. 15.

In an example where the signal 2200 represents an audible signal 1514, the signal 2200 is output as a sustained sound that changes pitch and/or volume in accordance with a waveform 2202 exhibiting the target duty cycle (e.g., a 40:60 duty cycle). In this sense, the signal 2200 is indicative of a target duty cycle of chest compressions (e.g., a 40:60 duty cycle). In the example of FIG. 22, a speaker(s) outputs the signal 2200 as a sustained sound, where the pitch and/or volume gradually decreases from a high pitch and/or volume to a low pitch and/or volume towards a middle of the duty cycle, and then ramps back up to the starting pitch and/or volume towards the end of the duty cycle. Alternatively, a speaker(s) can output an audible signal as a sustained sound, where the pitch and/or volume gradually increases from a low pitch and/or volume to a high pitch and/or volume towards the middle of the duty cycle, and then decays back down to the starting pitch and/or volume towards the end of the duty cycle. In another example, the pitch and the volume can vary inversely, whereby the volume gradually increases as the pitch gradually decreases, and vice versa. By outputting the signal 2200, the rescuer 204 is provided with duty cycle guidance that indicates, through changing pitch and/or volume of a sustained sound, when they should be compressing the chest of the subject 104, when the chest of the subject 104 should remain fully compressed, when they should be decompressing (e.g., releasing pressure applied to) the chest of the subject 104, and when the chest of the subject 104 should remain fully relaxed. In some examples, the hold periods 302 may be indicated by outputting the sustained sound at a constant pitch and/or volume.

In an example where the signal 2200 represents haptic signal 1516, the signal 2200 is output as a sustained vibration that changes frequency and/or amplitude in accordance with the waveform 2202 exhibiting the target duty cycle (e.g., a 40:60 duty cycle). In this sense, the signal 2200 is indicative of a target duty cycle of chest compressions (e.g., a 40:60 duty cycle). In the example of FIG. 22, a haptic actuator(s) outputs the signal 2200 as a sustained vibration, where the frequency and/or amplitude gradually decreases from a high frequency and/or amplitude to a low frequency and/or amplitude towards a middle of the duty cycle, and then ramps back up to the starting frequency and/or amplitude towards the end of the duty cycle. Alternatively, a haptic actuator(s) can output a haptic signal as a sustained vibration, where the frequency and/or amplitude gradually increases from a low frequency and/or amplitude to a high frequency and/or amplitude towards the middle of the duty cycle, and then decays back down to the starting frequency and/or amplitude towards the end of the duty cycle. In another example, the frequency and/or amplitude can vary inversely, whereby the amplitude gradually increases as the frequency gradually decreases, and vice versa. By outputting the signal 2200, the rescuer 204 is provided with duty cycle guidance that indicates, through changing frequency and/or amplitude of a sustained vibration, when they should be compressing the chest of the subject 104, when the chest of the subject 104 should remain fully compressed, when they should be decompressing (e.g., releasing pressure applied to) the chest of the subject 104, and when the chest of the subject 104 should remain fully relaxed. In some examples, the hold periods 302 may be indicated by the sustained vibration at a constant frequency and/or amplitude.

FIG. 23 illustrates an example signal(s) 2300 (audible and/or haptic) that may be output (via a speaker(s) and/or a haptic actuator(s)) to provide duty cycle guidance to a rescuer 204 who is manually administering chest compressions to the subject 104. The signal 2300 is an example of the audible signal(s) 1514 and/or the haptic signal(s) 1516 described above with reference to FIG. 15. The signal 2300 can be output via a speaker(s) and/or a haptic actuator(s) of any device of the CPR duty cycle guidance system 1502 (or the system 202 of FIG. 2) that includes a speaker(s) and/or a haptic actuator(s). For example, the signal 2300 can be output as an audible signal 1514 via a speaker(s) of the defibrillator 1504 and/or the signal 2300 can be output as a haptic signal 1516 via a haptic actuator(s) of the CPR feedback device 206 of FIG. 2 and/or a haptic actuator(s) of the wearable device 1510 of FIG. 15.

In an example where the signal 2300 represents an audible signal 1514, the signal 2300 is output as a first signal 2302 (e.g., sound) at a first time prior to a start of the compression phase 112 followed by a second signal 2304 (e.g., sound) at a second time prior to a start of the decompression phase 114. In this sense, the signal 2300 is indicative of a target duty cycle of chest compressions (e.g., a 40:60 duty cycle), the target duty cycle having a compression phase 112 and a decompression phase 114. The first signal 2302 (e.g., sound) may be output in advance of the start of the compression phase 112 to account for a delayed reaction of the rescuer 204, as indicated by the first time offset 2306 in FIG. 23. In other words, there may be latency between the rescuer 204 hearing the first signal 2302 (e.g., sound) and the rescuer 204 starting to compress the chest of the subject 104, and the first time offset 2306 accounts for that latency so that the rescuer 204 starts to compress the chest of the subject 104 at or near the start of the compression phase 112. The first time offset 2306 can be on the order of milliseconds, in some examples. Similarly, the second signal 2304 (e.g., sound) may be output in advance of the start of the decompression phase 114 to account for a delayed reaction of the rescuer 204, as indicated by the second time offset 2308 in FIG. 23, which may be the same as the first time offset 2306 or different than the first time offset 2306.

In some examples, the first time prior to the start of the compression phase 112 (e.g., the first time offset 2306) and the second time prior to the start of the decompression phase 114 (e.g., the second time offset 2308) are predetermined or fixed, such as predetermined time offsets 2306, 2308 on the order of several milliseconds. In some examples, the first time prior to the start of the compression phase 112 (e.g., the first time offset 2306) and the second time prior to the start of the decompression phase 114 (e.g., the second time offset 2308) are dynamically determined or adapted based on an observed reaction time of the rescuer 204, which can be determined from the administered duty cycle. For example, based on (e.g., by analyzing) the impedance signal generated using the electrode pads 1506 of the defibrillator 1504, as described above, the processor(s) 1312 may determine how long it has taken the rescuer 204 to react to the signals 2302, 2304 (e.g., sounds) in a previous compression-decompression cycle(s) (e.g., an average length of the rescuer's reaction time over the past N compression-decompressions cycles), and the processor(s) 1312 may dynamically determine the times (e.g., time offsets 2306, 2308) based on how long it takes for the rescuer 204 to react to the signals 2302, 2304 (e.g., sounds). As another example, the processor(s) 1312 may determine how long it takes for the rescuer 204 to react to the signals 2302, 2304 (e.g., sounds) based on (e.g., by analyzing) a compression parameter(s) sensed by a sensor(s) configured to be disposed on, or in proximity to, a chest of the subject 104, such as the sensor(s) 214 of the CPR feedback device 206 described above with reference to FIG. 2, and the processor(s) 1312 may dynamically determine the times (e.g., time offsets 2306, 2308) based on how long it takes for the rescuer 204 to react to the signals 2302, 2304 (e.g., sounds).

In an example where the signal 2300 represents a haptic signal 1516, the signal 2300 is output as a first signal 2302 (e.g., vibration) at a first time prior to a start of the compression phase 112 followed by a second signal 2304 (e.g., vibration) at a second time prior to a start of the decompression phase 114. In this sense, the signal 2300 is indicative of a target duty cycle of chest compressions (e.g., a 40:60 duty cycle), the target duty cycle having a compression phase 112 and a decompression phase 114. The first signal 2302 (e.g., vibration) may be output in advance of the start of the compression phase 112 to account for a delayed reaction of the rescuer 204, as indicated by the first time offset 2306 in FIG. 23. Similarly, the second signal 2304 (e.g., vibration) may be output in advance of the start of the decompression phase 114 to account for a delayed reaction of the rescuer 204, as indicated by the second time offset 2308 in FIG. 23, which may be the same as the first time offset 2306 or different than the first time offset 2306. In some examples, the first time prior to the start of the compression phase 112 (e.g., the first time offset 2306) and the second time prior to the start of the decompression phase 114 (e.g., the second time offset 2308) are predetermined or fixed, or they can be dynamically determined, as described above with respect to the audible signal example.

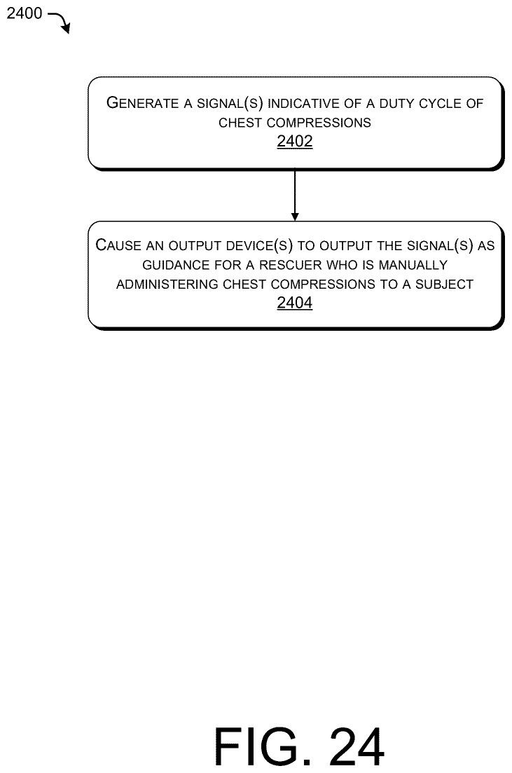

FIG. 24 illustrates an example process 2400 for providing, via a CPR duty cycle guidance system, guidance to a rescuer 204 who is manually administering chest compressions to the subject 104. The process 2400 is performed by an entity including, for example, one or more devices of a CPR duty cycle guidance system (e.g., the CPR feedback system 202 of FIG. 2, or the CPR duty cycle guidance system 1502 of FIG. 15), such as the defibrillator 208 of FIG. 2, the defibrillator 1300 of FIG. 13, the defibrillator 1504 of FIG. 15, the rescuer device 212 of FIG. 2, the rescuer device 1508 of FIG. 15, the wearable device 1510 of FIG. 15, the CPR feedback device 206 of FIG. 2, a processor(s) of any of the aforementioned devices and/or a processor(s) of any other device(s) that is part of a CPR system.

At 2402, the entity generates a signal(s) indicative of a duty cycle of chest compressions. The duty cycle represents a ratio of a first time period of a compression phase 112 of a compression-decompression cycle to a second time period of a decompression phase 114 of the compression-decompression cycle. In some examples, the signal(s) generated at 2402 is indicative of a target duty cycle (e.g., a duty cycle that is recommended as the optimal duty cycle for CPR treatment). In some examples, the target duty cycle includes a compression time period (the first time period) that is shorter than a decompression time period (the second time period). In some examples, the target duty cycle is within a range of about 35:65 to about 45:55. In some examples, the target duty cycle is about 40:60.

As discussed above, a non-50:50 target duty cycle with a compression phase 112 that is shorter than a decompression phase 114 not only reduces, if not eliminates, the wasted time period at the end of the compression phase 112 during which the heart is neither ejecting blood nor being filled with blood, but also creates extra time during the relatively longer decompression phase 114 during which the heart is able to fill with blood. Therefore, in some implementations, the target duty cycle is selected to increase cardiac filling, and, as a consequence, increase cardiac output and improve blood pressure, circulation, and perfusion in the subject 104 who is receiving manual CPR treatment.

In some examples, the signal(s) generated at 2402 is indicative of an administered duty cycle of chest compressions that the rescuer 204 manually administered to the subject 104. Examples of a signal(s) indicative of an administered duty cycle include the visual signal 1600 (e.g., impedance signal) depicted in FIG. 16, the second waveforms 1900(2), 1900(4), and 1900(6) depicted in FIGS. 19A-19C, respectively, the visual signal 2000 depicted in FIG. 20, and the visual signal 2100 depicted in FIGS. 21A and 21B.

In some examples, the signal(s) generated at 2402 includes a visual signal(s) 1512, an audible signal(s) 1514, and/or a haptic signal(s) 1516. For example, the entity may, at 2402, generate a visual signal(s) 1512 by itself, an audible signal(s) 1514 by itself, a haptic signal(s) 1516 by itself, a visual signal(s) 1512 and an audible signal(s) 1514, a visual signal(s) 1512 and a haptic signal(s) 1516, an audible signal(s) 1514 and a haptic signal(s) 1516, or a visual signal(s) 1512 and an audible signal(s) 1514 and a haptic signal(s) 1516.

At 2404, the entity causes an output device(s) to output the signal(s) generated at 2402 as guidance for a rescuer 204 who is manually administering the chest compressions to a subject 104. For example, the entity may cause, at 2404, an output device(s) to output one or more of the example signals described above with reference to FIGS. 15, 16, 17, 18, 19A-19C, 20, 21A, 21B, 22, and 23. These example signals provide the rescuer 204 with guidance by either indicating the target duty cycle that is recommended for administering the chest compressions to the subject 104, or indicating an administered duty cycle of the chest compressions the rescuer administered, or is administering, to the subject 104 (e.g., as a form of feedback for the rescuer 204), or both the target duty cycle and the administered duty cycle. If the signal(s) output at 2404 is a visual signal(s) 1512, the output device(s) may include a display(s), a light emitting element(s), and/or the like. If the signal(s) output at 2404 is an audible signal(s) 1514, the output device(s) may include a speaker(s). If the signal(s) output at 2404 is a haptic signal(s) 1516, the output device(s) may include a haptic actuator(s). If a combination of two or more different types of signals are output at 2404, the output device(s) may include a combination of two or more different types of output devices, such as a display(s), a speaker(s), and a haptic actuator(s).

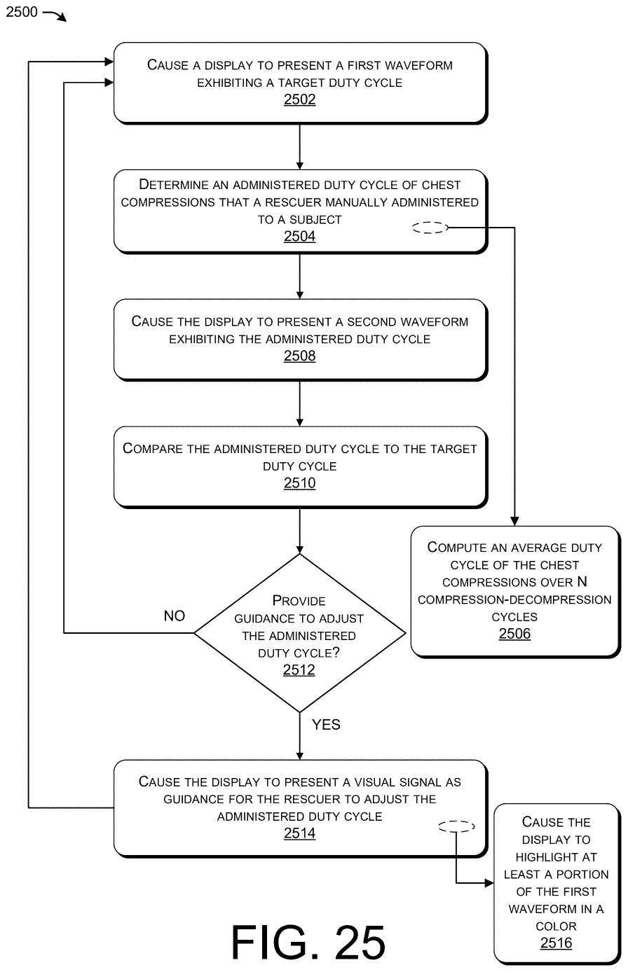

FIG. 25 illustrates an example process 2500 for providing, via a CPR duty cycle guidance system, visual guidance to a rescuer 204 who is manually administering chest compressions to the subject 104. The process 2500 is performed by an entity including, for example, one or more devices of a CPR duty cycle guidance system (e.g., the CPR feedback system 202 of FIG. 2, or the CPR duty cycle guidance system 1502 of FIG. 15), such as the defibrillator 208 of FIG. 2, the defibrillator 1300 of FIG. 13, the defibrillator 1504 of FIG. 15, the rescuer device 212 of FIG. 2, the rescuer device 1508 of FIG. 15, the wearable device 1510 of FIG. 15, the CPR feedback device 206 of FIG. 2, a processor(s) of any of the aforementioned devices and/or a processor(s) of any other device(s) that is part of a CPR system.

At 2502, the entity causes a display to present a first waveform exhibiting a target duty cycle. Examples of the first waveform presented at 2502 include the waveform included in the visual signal 1700 of FIG. 17, the waveform included in the visual signal 1800 of FIG. 18, and the first waveforms 1900(1), 1900(3), and 1900(5) depicted in FIGS. 19A-19C, respectively. In some examples, the first waveform is presented at 2502 with a moving icon that tracks along the first waveform. Examples of such a moving icon are depicted in FIGS. 17, 18, and 19A-19C.

At 2504, the entity determines an administered duty cycle of the chest compressions that the rescuer 204 manually administered, or is manually administering, to the subject 104. As shown by sub-block 2506, determining the administered duty cycle at 2504 may include computing an average duty cycle of the chest compressions that the rescuer 204 manually administered to the subject over N compression-decompression cycles, wherein N is an integer greater than one (e.g., N=5). With manual CPR, the duty cycle that the rescuer 204 uses for administering chest compressions can vary significantly over a short time period due to human error (e.g., the rescuer 204 may inadvertently change from 35:65 to 45:55 over the span of five compression-decompression cycles while attempting to deliver manual chest compressions at a fixed duty cycle (e.g., 40:60)). In this example, the average (or mean) duty cycle over the past N compression-decompression cycles that is computed at 2506 may be a 40:60 duty cycle, notwithstanding some observed variability in the administered duty cycle from one compression-decompression cycle to the next. Accordingly, determining the administered duty cycle by computing the average duty cycle at 2506 can allow for providing guidance to the rescuer 204 that is more stable and does not switch rapidly back-and-forth between different types of guidance.

In some examples, the entity determines the administered duty cycle at 2504 based on (e.g., by analyzing) an impedance signal generated using electrode pads of a defibrillator, as described above. In some examples, the entity determines the administered duty cycle at 2504 based on (e.g., by analyzing) a compression parameter(s) sensed by a sensor(s) configured to be disposed on, or in proximity to, a chest of the subject 104, such as the sensor(s) 214 of the CPR feedback device 206 described above with reference to FIG. 2. In some examples, the entity uses a sensor fusion approach that analyzes both the impedance signal generated using the electrode pads of the defibrillator and the compression parameter(s) sensed by the CPR feedback device 206. In this sensor fusion approach, the entity may use the impedance signal generated using the electrode pads to corroborate the compression parameter(s) sensed by the CPR feedback device 206, or vice versa. In some examples, a first administered duty cycle is determined based on the impedance signal generated using the electrode pads and a second administered duty cycle is determined based on the compression parameter(s) sensed by the CPR feedback device 206, and if the first administered duty cycle is different than the second administered duty cycle, the entity may select the first administered duty cycle or the second administered duty cycle based on which of the two signal sources is more reliable, and/or based on a confidence score(s), and/or the like. In some examples, the entity selects a mode for determining the administered duty cycle based on whether a CPR feedback device 206 is communicatively coupled to a defibrillator or not. For example, if the entity determines that a CPR feedback device 206 is communicatively coupled to a defibrillator, the entity may select a “CPR feedback device mode” where the administered duty cycle is determined at 2504 based on a compression parameter(s) sensed by a sensor(s) of the CPR feedback device 206. On the other hand, if the entity determines that a CPR feedback device 206 is not communicatively coupled to a defibrillator, the entity may select an “impedance mode” where the administered duty cycle is determined at 2504 based on the impedance signal, as described above.

At 2508, the entity causes the display to present a second waveform exhibiting the administered duty cycle determined at 2504. Examples of the second waveform presented at 2508 include the waveform included in the visual signal 1600 (e.g., impedance signal) of FIG. 16, and the second waveforms 1900(2), 1900(4), and 1900(6) depicted in FIGS. 19A-19C, respectively. In some examples, the second waveform is overlaid on top of the first waveform and aligned with the first waveform on at least a time axis. Examples of such overlaid waveforms are depicted in FIGS. 19A-19C.

At 2510, the entity compares the administered duty cycle determined at 2504 to the target duty cycle (e.g., a 40:60 duty cycle). For example, the administered duty cycle and the target duty cycle can be aligned on a time axis and the entity can determine a difference, if any, of the respective times at which a transition from the compression phase 112 to the decompression phase 114 occurs for the administered duty cycle and the target duty cycle, respectively. In some examples, the comparison at 2510 includes a comparison of respective waveforms that plot the position (e.g., a position of a point on a surface of a chest of the subject 104) over time for one or more compression-decompression cycles, and the entity may determine differences, if any, between respective positions in the respective waveforms at particular time points.

At 2512, the entity determines whether to provide guidance to the rescuer 204 to adjust the administered duty cycle. For example, if the administered duty cycle substantially matches the target duty cycle (e.g., if the differences mentioned above are less than a threshold difference), the entity may determine to refrain from providing guidance to the rescuer 204 to adjust the administered duty cycle, and the process 2500 follows the NO route from 2512 back to 2502 to iterate blocks 2502-2510. If the administered duty cycle is different from the target duty cycle by more than a threshold difference, the entity may determine to provide guidance to the rescuer 204 to adjust the administered duty cycle, and the process 2500 follows the YES route from 2512 to 2514.

At 2514, the entity causes the display to present a visual signal(s) 1512 as guidance for the rescuer 204 to adjust the administered duty cycle. Examples of such a visual signal(s) 1512 include the visual signal 1906 depicted in FIG. 19B, the text 1908 depicted in FIG. 19C, the visual signal 2000 and/or the text 2002 depicted in FIG. 20, and the visual signal 2100(B) depicted in FIG. 21B. As shown by sub-block 2516, causing the display to present a visual signal(s) 1512 at 2514 can include causing the display to highlight at least a portion of the first waveform (and/or the second waveform) in a color (e.g., in the color red) as guidance for the rescuer 204 to adjust the administered duty cycle. As discussed above, the visual signal(s) 1512 presented as guidance for the rescuer 204 to adjust the administered duty cycle may include other visual indicia, such as an arrow pointing in a particular direction to indicate whether to shorten or lengthen the compression phase 112 or the decompression phase 114 of the administered duty cycle. As shown by the return arrow from 2514 back to 2502, the process 2500 may iterate as the rescuer 204 continues administering manual CPR.

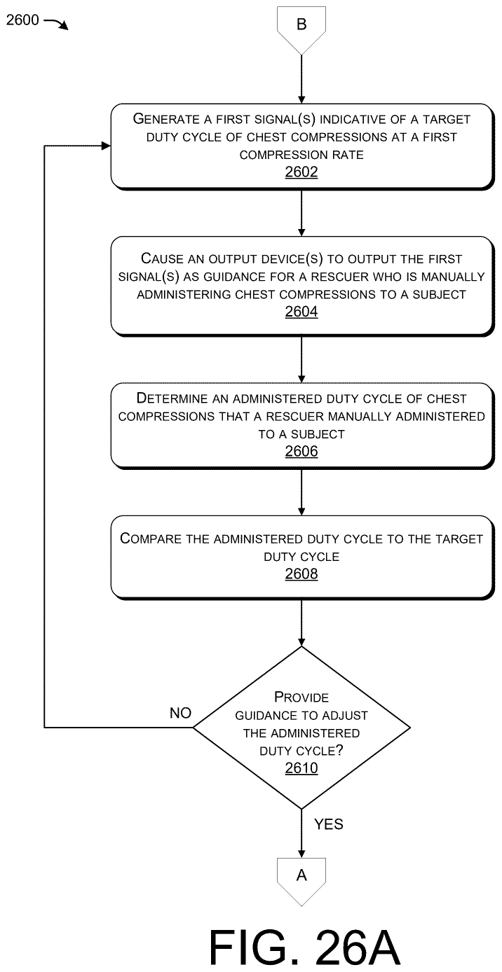

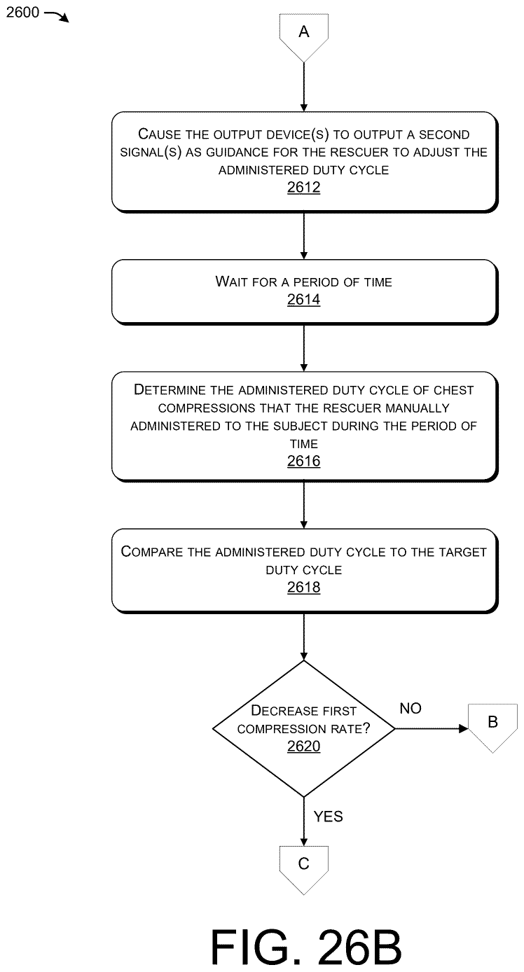

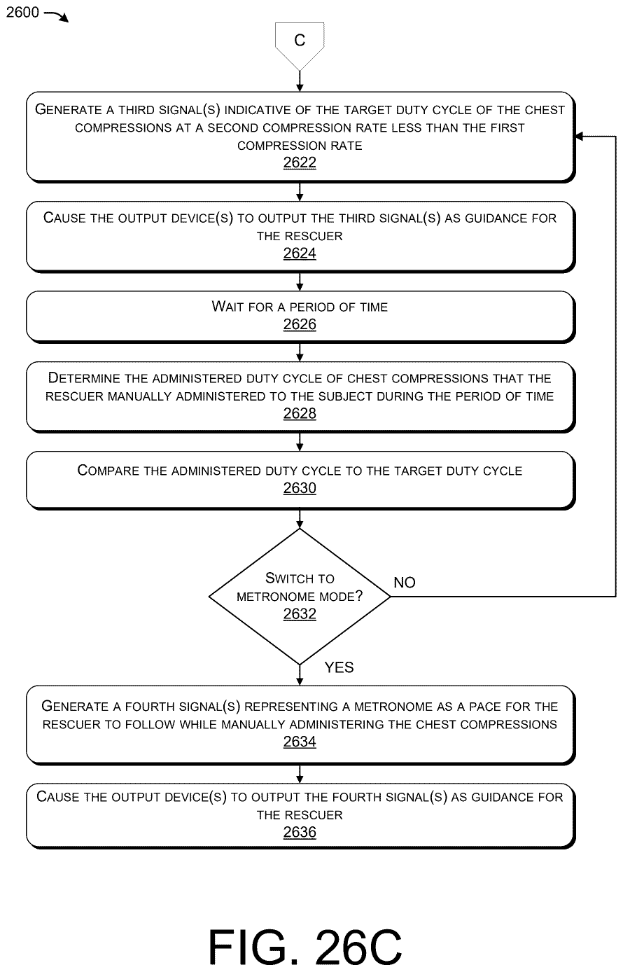

FIGS. 26A-26C illustrate an example process 2600 for providing, via a CPR duty cycle guidance system, guidance to a rescuer 204 who is manually administering chest compressions to the subject 104. The process 2600 is performed by an entity including, for example, one or more devices of a CPR duty cycle guidance system (e.g., the CPR feedback system 202 of FIG. 2, or the CPR duty cycle guidance system 1502 of FIG. 15), such as the defibrillator 208 of FIG. 2, the defibrillator 1300 of FIG. 13, the defibrillator 1504 of FIG. 15, the rescuer device 212 of FIG. 2, the rescuer device 1508 of FIG. 15, the wearable device 1510 of FIG. 15, the CPR feedback device 206 of FIG. 2, a processor(s) of any of the aforementioned devices and/or a processor(s) of any other device(s) that is part of a CPR system.

At 2602, the entity generates a first signal(s) indicative of a target duty cycle of chest compressions at a first compression rate. The first compression rate can be any suitable compression rate, such as 100 compressions per minute, 110 compressions per minute, 115 compressions per minute, or the like. In some examples, the first signal(s) generated at 2602 includes a visual signal(s) 1512, an audible signal(s) 1514, and/or a haptic signal(s) 1516. For example, the entity may, at 2602, generate a visual signal(s) 1512 by itself, an audible signal(s) 1514 by itself, a haptic signal(s) 1516 by itself, a visual signal(s) 1512 and an audible signal(s) 1514, a visual signal(s) 1512 and a haptic signal(s) 1516, an audible signal(s) 1514 and a haptic signal(s) 1516, or a visual signal(s) 1512 and an audible signal(s) 1514 and a haptic signal(s) 1516.

At 2604, the entity causes an output device(s) to output the first signal(s) generated at 2602 as guidance for a rescuer 204 who is manually administering the chest compressions to a subject 104. For example, the entity may cause, at 2604, an output device(s) to output one or more of the example signals described above with reference to FIGS. 15, 16, 17, 18, 19A-19C, 20, 21A, 21B, 22, and 23 that are indicative of a target duty cycle of chest compressions. If the first signal(s) output at 2604 is a visual signal(s) 1512, the output device(s) may include a display(s), a light emitting element(s), and/or the like. If the first signal(s) output at 2604 is an audible signal(s) 1514, the output device(s) may include a speaker(s). If the first signal(s) output at 2604 is a haptic signal(s) 1516, the output device(s) may include a haptic actuator(s). If a combination of two or more different types of first signals are output at 2604, the output device(s) may include a combination of two or more different types of output devices, such as a display(s), a speaker(s), and a haptic actuator(s).

At 2606, the entity determines an administered duty cycle of the chest compressions that the rescuer 204 manually administered, or is manually administering, to the subject 104. The operation(s) performed at 2606 may be the same as, or similar to, the operation(s) described above with reference to block 2504 of the process 2500 of FIG. 25.

At 2608, the entity compares the administered duty cycle determined at 2606 to the target duty cycle (e.g., a 40:60 duty cycle). The operation(s) performed at 2608 may be the same as, or similar to, the operation(s) described above with reference to block 2510 of the process 2500 of FIG. 25.

At 2610, the entity determines whether to provide guidance to the rescuer 204 to adjust the administered duty cycle. For example, if the administered duty cycle substantially matches the target duty cycle, the entity may determine to refrain from providing guidance to the rescuer 204 to adjust the administered duty cycle, and the process 2600 follows the NO route from 2610 back to 2602 to iterate blocks 2602-2608. If the administered duty cycle is different from the target duty cycle by more than a threshold difference, the entity may determine to provide guidance to the rescuer 204 to adjust the administered duty cycle, and the process 2600 follows the YES route from 2610 to 2612 (via off-page reference “A” in FIGS. 26A and 26B).

Turning to FIG. 26B, at 2612, the entity causes the output device(s) to output a second signal(s) as guidance for the rescuer 204 to adjust the administered duty cycle. In some examples, the second signal(s) output at 2612 includes a visual signal(s) 1512, an audible signal(s) 1514, and/or a haptic signal(s) 1516. Examples of a visual signal(s) 1512 that can be output as guidance for the rescuer 204 to adjust the administered duty cycle include the visual signal 1906 depicted in FIG. 19B, the text 1908 depicted in FIG. 19C, the visual signal 2000 and/or the text 2002 depicted in FIG. 20, and the visual signal 2100(B) depicted in FIG. 21B. In some examples, the output device(s) includes a display, and the entity, at 2612, causes the display to present the second signal(s) as a visual signal(s) 1512 that highlights at least a portion of the waveform exhibiting a duty cycle in a color (e.g., in the color red) as guidance for the rescuer 204 to adjust the administered duty cycle. As discussed above, a visual signal(s) 1512 presented as guidance for the rescuer 204 to adjust the administered duty cycle may include other visual indicia, such as an arrow pointing in a particular direction to indicate whether to shorten or lengthen the compression phase 112 or the decompression phase 114 of the administered duty cycle. In some examples, the second signal(s) output at 2612 includes an audible signal(s) 1514 in the form of verbal instructions (e.g., a synthetic voice that utters the instruction “Shorten the compression phase!”).

At 2614, the entity waits for a period of time. The period of time can be any suitable period of time, such as a few seconds, which provides the rescuer 204 with some time to adjust the administered duty cycle.

At 2616, the entity determines the administered duty cycle of the chest compressions that the rescuer 204 manually administered, or is manually administering, to the subject 104 during the period of time at 2614. The operation(s) performed at 2616 may be the same as, or similar to, the operation(s) described above with reference to block 2504 of the process 2500 of FIG. 25.

At 2618, the entity compares the administered duty cycle determined at 2616 to the target duty cycle (e.g., a 40:60 duty cycle). The operation(s) performed at 2618 may be the same as, or similar to, the operation(s) described above with reference to block 2510 of the process 2500 of FIG. 25.

At 2620, the entity determines whether to decrease the first compression rate. For example, if the administered duty cycle substantially matches the target duty cycle, indicating that the rescuer 204 adjusted the administered duty cycle to be in substantial alignment with the target duty cycle, the entity may determine to refrain from decreasing the first compression rate, and the process 2600 follows the NO route from 2620 back to 2602 (via off-page reference “B” in FIGS. 26A and 26B) to iterate blocks 2602-2608. If the administered duty cycle is different from the target duty cycle by more than a threshold difference, the entity may determine to decrease the first compression rate, and the process 2600 follows the YES route from 2620 to 2622 (via off-page reference “C” in FIGS. 26B and 26C).

Turning to FIG. 26C, at 2622, the entity generates a third signal(s) indicative of the target duty cycle of chest compressions at a second compression rate less than the first compression rate. For example, if the first compression rate is 110 compressions per minute, the second compression rate may be 100 compressions per minute. Decreasing the compression rate in this manner can help the rescuer 204 adjust the administered duty cycle because the chest compressions are administered by the rescuer 204 at a lower compression rate and it may be easier for the rescuer 204, at the lower compression rate, to follow along with the guidance that is to be provided by the third signal(s). In some examples, the third signal(s) generated at 2622 includes a visual signal(s) 1512, an audible signal(s) 1514, and/or a haptic signal(s) 1516. For example, the entity may, at 2622, generate a visual signal(s) 1512 by itself, an audible signal(s) 1514 by itself, a haptic signal(s) 1516 by itself, a visual signal(s) 1512 and an audible signal(s) 1514, a visual signal(s) 1512 and a haptic signal(s) 1516, an audible signal(s) 1514 and a haptic signal(s) 1516, or a visual signal(s) 1512 and an audible signal(s) 1514 and a haptic signal(s) 1516.

At 2624, the entity causes the output device(s) to output the third signal(s) generated at 2622 as guidance for a rescuer 204 who is manually administering the chest compressions to a subject 104. For example, the entity may cause, at 2624, the output device(s) to output one or more of the example signals described above with reference to FIGS. 15, 16, 17, 18, 19A-19C, 20, 21A, 21B, 22, and 23 that are indicative of a target duty cycle of chest compressions.

At 2626, the entity waits for a period of time. The period of time can be any suitable period of time, such as a few seconds, which provides the rescuer 204 with some time to align the administered duty cycle with the target duty cycle at the second (decreased) compression rate.

At 2628, the entity determines the administered duty cycle of the chest compressions that the rescuer 204 manually administered, or is manually administering, to the subject 104 during the period of time at 2626. The operation(s) performed at 2628 may be the same as, or similar to, the operation(s) described above with reference to block 2504 of the process 2500 of FIG. 25.

At 2630, the entity compares the administered duty cycle determined at 2628 to the target duty cycle (e.g., a 40:60 duty cycle). The operation(s) performed at 2630 may be the same as, or similar to, the operation(s) described above with reference to block 2510 of the process 2500 of FIG. 25.

At 2632, the entity determines whether to switch to a fallback mode (e.g., a “metronome mode”) of operation. For example, if the administered duty cycle substantially matches the target duty cycle, indicating that the rescuer 204 is administering chest compressions at or near the target duty cycle, the entity may determine to refrain from switching to the fallback mode, and the process 2600 follows the NO route from 2632 back to 2622 to iterate blocks 2622-2630. If the administered duty cycle is different from the target duty cycle by more than a threshold difference, the entity may determine to switch to the fallback mode, and the process 2600 follows the YES route from 2632 to 2634.

At 2634, the entity generates a fourth signal(s) representing a metronome as a pace for the rescuer 204 to follow while manually administering the chest compressions. The metronome may be a fallback approach to guiding the rescuer 204 if it is determined, via execution of the process 2600, that the rescuer 204 is having trouble administering chest compressions at or near the target duty cycle notwithstanding the guidance provided to the rescuer 204 up to the point of determining that the rescuer 204 is having trouble adhering closely enough to the target duty cycle. In some examples, the metronome signal generated at 2634 is an audible signal(s) and/or a haptic signal(s) that includes repeating sounds and/or vibrations at a constant frequency corresponding to the compression rate (e.g., the second compression rate).

At 2636, the entity causes the output device(s) to output the fourth signal(s) as guidance for the rescuer 204. In some examples, the output device(s) includes a speaker(s) and/or a haptic actuator(s).

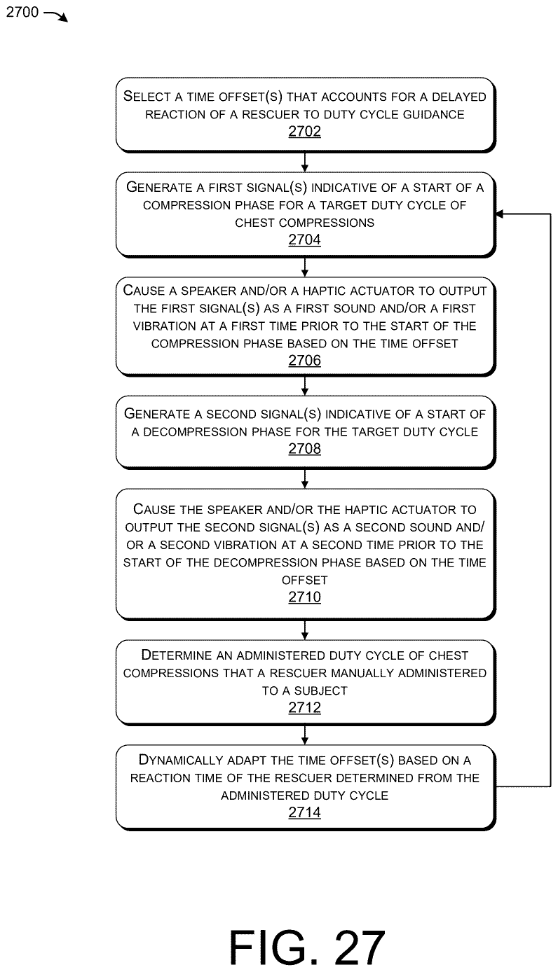

FIG. 27 illustrates an example process 2700 for providing, via a CPR duty cycle guidance system, audible and/or haptic guidance to a rescuer 204 who is manually administering chest compressions to the subject 104. The process 2700 is performed by an entity including, for example, one or more devices of a CPR duty cycle guidance system (e.g., the CPR feedback system 202 of FIG. 2, or the CPR duty cycle guidance system 1502 of FIG. 15), such as the defibrillator 208 of FIG. 2, the defibrillator 1300 of FIG. 13, the defibrillator 1504 of FIG. 15, the rescuer device 212 of FIG. 2, the rescuer device 1508 of FIG. 15, the wearable device 1510 of FIG. 15, the CPR feedback device 206 of FIG. 2, a processor(s) of any of the aforementioned devices and/or a processor(s) of any other device(s) that is part of a CPR system.

At 2702, the entity selects a time offset(s) that accounts for a delayed reaction of a rescuer 204 to duty cycle guidance. For example, as mentioned above, there may be latency between a time at which the rescuer 204 hears/feels an audible/haptic signal(s) and a time at which the rescuer 204 starts to compress, or release a compression of, the chest of the subject 104. The time offset(s) selected at 2702 can be predetermined and can be on the order of several milliseconds, in some examples. In some examples, the entity selects multiple (e.g., two) different time offsets at 2702, where a first time offset 2306 is used for outputting an audible signal and/or a haptic signal in advance of the compression phase 112, and a second time offset 2308 is used for outputting an audible signal and/or a haptic signal in advance of the decompression phase 114. In some examples, the first time offset 2306 and the second time offset 2308 are equal and/or the entity selects a single time offset at 2702 that is used for outputting an audible signal and/or a haptic signal in advance of each of the compression phase 112 and the decompression phase 114.

At 2704, the entity generates a first signal(s) 2302 indicative of a start of a compression phase 112 for a target duty cycle of chest compressions, such as a 40:60 duty cycle. The first signal(s) 2302 can include an audible signal 1514 and/or a haptic signal 1516.

At 2706, the entity causes a speaker(s) and/or a haptic actuator(s) to output the first signal(s) 2302 as a first sound and/or a first vibration at a first time prior to the start of the compression phase 112 based on the time offset (e.g., first time offset 2306) selected at 2702. In some examples, because the time offset (e.g., first time offset 2306) selected at 2702 is predetermined, the first time prior to the start of the compression phase 112 is a first predetermined time prior to the start of the compression phase 112. Outputting the first signal(s) 2302 in advance of the start of the compression phase 112 accounts for a delayed reaction of the rescuer 204 so that the rescuer 204 starts compressing the chest of the subject 104 at the start of the compression phase 112.

At 2708, the entity generates a second signal(s) 2304 indicative of a start of a decompression phase 114 for the target duty cycle. The second signal(s) 2304 can include an audible signal 1514 and/or a haptic signal 1516.

At 2710, the entity causes the speaker(s) and/or the haptic actuator(s) to output the second signal(s) 2304 as a second sound and/or a second vibration at a second time prior to the start of the decompression phase 114 based on the time offset (e.g., second time offset 2308) selected at 2702. In some examples, because the time offset (e.g., second time offset 2308) selected at 2702 is predetermined, the second time prior to the start of the decompression phase 114 is a second predetermined time prior to the start of the decompression phase 114. Outputting the second signal(s) 2304 in advance of the start of the decompression phase 114 accounts for a delayed reaction of the rescuer 204 so that the rescuer 204 starts releasing pressure applied to the chest of the subject 104 at the start of the decompression phase 114.

At 2712, the entity determines an administered duty cycle of the chest compressions that the rescuer 204 manually administered, or is manually administering, to the subject 104. The operation(s) performed at 2712 may be the same as, or similar to, the operation(s) described above with reference to block 2504 of the process 2500 of FIG. 25.

At 2714, the entity dynamically adapts or adjusts the time offset(s) based on an observed reaction time of the rescuer 204 determined from the administered duty cycle. For example, if the compression phase 112 of the administered duty cycle started after the start of the compression phase 112 of the target duty cycle, the entity may dynamically increase the time offset (e.g., first time offset 2306). Conversely, if the compression phase 112 of the administered duty cycle started before the start of the compression phase 112 of the target duty cycle, the entity may dynamically decrease the time offset (e.g., first time offset 2306). Similarly, if the decompression phase 114 of the administered duty cycle started after the start of the decompression phase 114 of the target duty cycle, the entity may dynamically increase the time offset (e.g., second time offset 2308). Conversely, if the decompression phase 114 of the administered duty cycle started before the start of the decompression phase 114 of the target duty cycle, the entity may dynamically decrease the time offset (e.g., second time offset 2308). As shown by the return arrow from 2714 back to 2704, the process 2700 may iterate blocks 2704-2714 with the adjusted time offset(s). In this manner, over multiple iterations of the process 2700, the time offset(s) may self-calibrate by converging towards a time offset(s) that is appropriate for the specific rescuer 204.

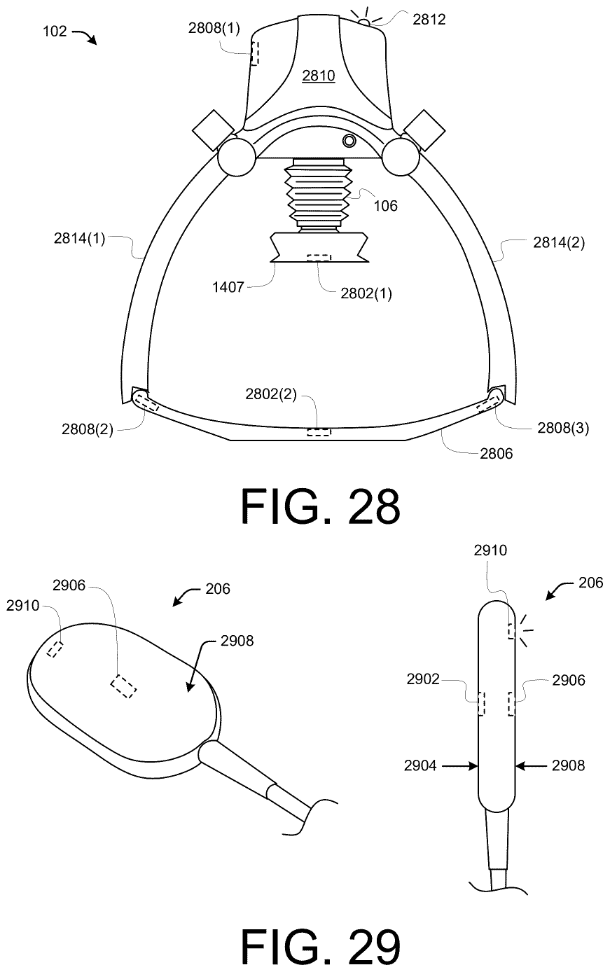

FIG. 28 illustrates the example mechanical chest compression device 102 depicted in FIG. 1, FIG. 28 depicting various example components of the mechanical chest compression device 102 to, among other things, record sounds (e.g., internal body sounds, environment sounds, etc.) and dynamically adjust a duty cycle of a mechanical chest compression device 102. In the example of FIG. 28, the mechanical chest compression device 102 includes a first auscultation device 2802(1) disposed on a distal end of the chest compressing mechanism 106, which is a piston (e.g., piston 1405) in the example of FIG. 28. The distal end of the chest compressing mechanism 106 (e.g., piston 1405) is configured to contact a chest of the subject 104 when the mechanical chest compression device 102 is secured around the subject 104 (e.g., around a thorax thereof). In an example, the distal end of the chest compressing mechanism 106 (e.g., piston 1405) may include a suction cup 1407 that is configured to adhere to the chest of the subject 104 in order to actively decompress the chest by pulling upward on the chest. In this example, the first auscultation device 2802(1) is disposed on the suction cup 1407. Because the first auscultation device 2802(1) is disposed on the distal end of the chest compressing mechanism 106, the first auscultation device 2802(1) is configured to be disposed on a thorax of the subject 104, and specifically on the chest of the subject 104. Accordingly, the first auscultation device 2802(1) is configured to detect a heart sound associated with the subject 104. In the example of FIG. 28, the mechanical chest compression device 102 additionally, or alternatively, includes a second auscultation device 2802(2) disposed on an inner surface of a backplate 2806 of the mechanical chest compression device 102. FIG. 28 shows that the second auscultation device 2802(2) as being positioned substantially in the center of the backplate 2806, but the second auscultation device 2802(2) can be positioned away from the center and closer to the sides of the backplate 2806, so long as the second auscultation device 2802(2) is positioned where it will contact the back of the subject 104 when the mechanical chest compression device 102 is secured around the subject 104. Because the backplate 2806 is configured to be disposed under the back of the subject 104, and because the second auscultation device 2802(2) is disposed on an inner surface of the backplate 2806, the second auscultation device 2802(2) is configured to be disposed on a thorax of the subject 104, and specifically on the back of the subject 104. Accordingly, the second auscultation device 2802(2) is configured to detect a heart sound associated with the subject 104. In an example, the first auscultation device 2802(1) is included in the mechanical chest compression device 102 and the second auscultation devices 2802(2) is excluded or omitted from the mechanical chest compression device 102. In another example the second auscultation device 2802(2) is included in the mechanical chest compression device 102 and the first auscultation device 2802(1) is excluded or omitted from the mechanical chest compression device 102. In another example, both the first auscultation device 2802(1) and the second auscultation device 2802(2) are included in the mechanical chest compression device 102. In this example, two heart sound signals from two different sources are obtained and, if combined, a more reliable heart sound signal is obtained. In an example, the auscultation device 2802 (the first auscultation device 2802(1) and/or the second auscultation device 2802(2)) includes an array of auscultation devices 2802 with multiple measurement sources spread out on a surface in contact to the subject 104 where several measurement signals are obtained, and if combined, the Signal-to-Noise (SNR) ratio may be improved.

The auscultation devices 2802 can be implemented in various ways. In some examples, an auscultation device is, or includes, a microphone, a stethoscope (e.g., analog or digital), or any other suitable type of audio sensor. In some examples, an auscultation device is, or includes, a force sensor, a pressure sensor and/or the like. In some examples, the auscultation devices 2802 include an analog to digital converter (ADC) and/or a transducer to convert detected heart sound signals into digital signals indicative of heart sound. In some examples, the auscultation devices 2802 include components configured to magnify and/or amplify the detected sound, such as a bell and diaphragm in a stethoscope implementation. In some examples, upon detecting heart sound, the auscultation devices 2802 can sample the heart sound to obtain a digital audio signal. In some examples, a microphone is used to sample the heart sound and to obtain a digital audio signal. Any suitable type of microphone can be used as part of the auscultation devices 2802, such as dynamic microphones, condenser microphones, ribbon microphones, carbon microphones, crystal microphones, and/or the like.

In the example of FIG. 28, the mechanical chest compression device 102 additionally, or alternatively, includes a microphone 2808(1) disposed on an external surface of the mechanical chest compression device 102. Although FIG. 28 shows the microphone 2808(1) as being disposed on a main portion 2810 of the mechanical chest compression device 102, the microphone 2808(1) can be disposed elsewhere on any external surface of the mechanical chest compression device 102. The microphone 2808(1) is configured to detect a sound in an environment (e.g., the environment 100) where the mechanical chest compression device 102 is located while chest compressions are being administered to the subject 104. By using the microphone 2808(1) to detect environment sound, the processor(s) 1414 of the mechanical chest compression device 102 may be configured to remove the sound in the environment detected by one or both of the auscultation devices 2802(1), 2802(2), such as by performing active-noise-cancellation (ANC) to remove the environment sound. For example, the processor(s) 1414 may receive an audio signal(s) representing a heart sound detected by one or both of the auscultation devices 2802(1), 2802(2), and the processor(s) 1414 may remove, from the audio signal, sound (e.g., environment sound) detected by the microphone 2808(1). In some examples, the processor(s) 1414 of the mechanical chest compression device 102 may be configured to perform signal averaging over multiple automated chest compressions administered to the subject 104. For example, the processor(s) 1414 may be configured to average a gated portion of audio data generated using the auscultation device(s) 2802 and/or the microphone(s) 2808 over a predefined analysis window (e.g., a 10-second analysis window during an end of the compression phase and a start of the decompression phase of a compression-decompression cycle). This can improve the SNR of the audio data generated in association with the automated chest compressions In some examples, sounds within the chest of the subject 104, as recorded by the auscultation device(s) 2802, can be used to adjust a CPR parameter(s) (e.g., duty cycle, compression position, compression depth, compression rate, etc.) and/or to cause a feedback signal(s) to be output to change the CPR parameter(s). In some examples, these “chest sounds” may exhibit the sound(s) of a heart valve(s) opening and/or closing, and/or other sounds associated with blood flow (e.g., blood flow through (e.g., exiting from) the aortic valve, which may be turbulent blood flow). For example, rules and/or statistical correlations can be generated based on an analysis of heart sounds during CPR to correlate particular chest sounds with “good” blood flow and other chest sounds with “poor” blood flow (e.g., a blood flow parameter that satisfies, or fails to satisfy, a threshold value).

Additionally, or alternatively, the microphone 2808(1) can be used to record a monologue of a rescuer or a dialogue between multiple rescuers (or bystanders) at a rescue scene, which may be helpful for offline review of CPR quality, to record notes of a rescuer without the rescuer having to instruct an assistant to record hand-written notes in real-time, and/or the like. For example, rescuers may have conversations at a rescue scene while the mechanical chest compression device 102 is administering automated chest compressions, and a post review process may utilize recordings of these conversations to improve CPR training, to perform audits, and/or for legal reasons. In another example, a rescuer may speak into the microphone 2808(1) to record notes in a hands-free manner (e.g., without having to use a separate recording device).

In the example of FIG. 28, the mechanical chest compression device 102 additionally, or alternatively, includes a light emitting element(s) 2812, such as a LED(s), configured to emit light of various wavelengths or colors. Although FIG. 28 shows the light emitting element(s) 2812 as being disposed on a main portion 2810 of the mechanical chest compression device 102, the light emitting element(s) 2812 can be disposed elsewhere on any external surface of the mechanical chest compression device 102. The light emitting element(s) 2812 is configured to provide a visual feedback signal(s) as feedback relating to the duty cycle. For example, the light emitting element(s) 2812 may emit red-colored light to indicate that the duty cycle should be changed, or a green-colored light to indicate that the duty cycle is correct. In some examples, the light emitting element(s) 2812 may flash to indicate that the duty cycle is incorrect, and may emit a sustained light, or no light, to indicate that the duty cycle is correct. These are merely examples and the light emitting element(s) 2812 may be configured to provide visual feedback relating to the duty cycle in other ways.

In the example of FIG. 28, the mechanical chest compression device 102 additionally, or alternatively, includes a second microphone 2808(2) and/or a third microphone 2808(3). The second microphone 2808(2) is disposed in, or on, the backplate 2806 near a first side of the backplate 2806 that is configured to connect to a first leg 2814(1) of a pair of legs 2814 of the mechanical chest compression device 102. For example, the mechanical chest compression device 102 may include a pair of legs 2814 extending from the main portion 2810, each leg 2814(1), 2814(2) of the pair of legs 2814 configured to connect to a respective side of the backplate 2806. Accordingly, the second microphone 2808(2), by being disposed near the first side of the backplate 2806, is configured to detect an audible connection sound indicating that the first leg 2814(1) has been connected to the backplate 2806. Additionally, or alternatively, the third microphone 2808(3) is disposed in, or on, the backplate 2806 near a second side of the backplate 2806 that is configured to connect to a second leg 2814(2) of the pair of legs 2814. Accordingly, the third microphone 2808(3), by being disposed near the second side of the backplate 2806, is configured to detect an audible connection sound indicating that the second leg 2814(2) has been connected to the backplate 2806. In some examples, one or both of the microphones 2808(2), 2808(3) can be used to detect an audible connection sound indicating that the pair of legs 2814 have been connected to the backplate 2806. In some examples, operation of the mechanical chest compression device 102 is disabled until the processor(s) 1414 receives an indication that the microphone(s) 2808(2) and/or 2808(3) detected an audible connection sound, which may prevent operation of the mechanical chest compression device 102 while the legs 2814 are not securely connected to the backplate 2806. Accordingly, the processor(s) 1414 may be configured to enable operation of the mechanical chest compression device 102 in response to receiving an indication that the audible connection sound(s) was detected by the microphone(s) 2808(2) and/or 2808(3), which ensures that the mechanical chest compression device 102 remains securely positioned on and/or around the subject 104 during a CPR session.

FIG. 29 illustrates the example CPR feedback device 206 depicted in FIG. 2, FIG. 29 depicting various example components of the CPR feedback device 206 to, among other things, record sounds (e.g., internal body sounds, environment sounds, etc.) and to output a signal(s) to provide duty cycle guidance to a rescuer 204 who is manually administering chest compressions to the subject 104. In the example of FIG. 29, the CPR feedback device 206 includes an auscultation device 2902 disposed on an inner surface 2904 of the CPR feedback device 206 that is configured to contact the chest of a subject 104 when the CPR feedback device 206 is placed on chest. Accordingly, the auscultation device 2902 is configured to detect a heart sound associated with the subject 104 as manual chest compressions are being administered to the subject 104. The auscultation device 2902 may be implemented in the various ways described above with respect to the auscultation devices 2802 of FIG. 28. In an example, the auscultation device 2902 is not part of the enclosure (e.g., the inner surface 2904) of the CPR feedback device 206, but rather includes an external part connected (via wire or wireless) to the CPR feedback device 206. In another example, the external part may include more than one auscultation device 2902 integrated in one or more external parts. In another example, the external part may include a belt including one or more auscultation devices 2902 configured to be wrapped around the chest of the subject 104.

In the example of FIG. 29, the CPR feedback device 206 additionally, or alternatively, includes a microphone 2906 disposed on an outer surface 2908 of the CPR feedback device 206. The microphone 2906 is configured to detect a sound in an environment (e.g., the environment 200) where the CPR feedback device 206 is located while chest compressions are being administered to the subject 104 by the rescuer 204. By using the microphone 2906 to detect environment sound, a processor(s) of the CPR feedback system 202 may be configured to remove the sound in the environment detected by the auscultation device 2902, such as by performing ANC to remove the environment sound. For example, the processor(s) of the CPR feedback system 202 may receive an audio signal(s) representing a heart sound detected by the auscultation device 2902, and the processor(s) may remove, from the audio signal, sound (e.g., environment sound) detected by the microphone 2906. Additionally, or alternatively, the microphone 2906 can be used to record a monologue of a rescuer 204 or a dialogue between multiple rescuers (or bystanders) at a rescue scene, which may be helpful for offline review of CPR quality, to record notes of a rescuer 204 without the rescuer having to instruct an assistant to record hand-written notes in real-time, and/or the like. For example, rescuers may have conversations at a rescue scene while the CPR feedback device 206 is being used in association with the administration of manual chest compressions, and a post review process may utilize recordings of these conversations to improve CPR training, to perform audits, and/or for legal reasons. In another example, a rescuer 204 may speak into the microphone 2906 to record notes in a hands-free manner (e.g., without having to use a separate recording device). In an example, the microphone 2906 is not part of the enclosure (e.g., the outer surface 2908) of the CPR feedback device 206, but rather includes an external part connected (via wire or wireless) to the CPR feedback device 206.

In the example of FIG. 29, the CPR feedback device 206 additionally, or alternatively, includes a light emitting element(s) 2910, such as a LED(s), configured to emit light of various wavelengths or colors. The light emitting element(s) 2910 is configured to provide a visual feedback signal(s) as feedback relating to the duty cycle. For example, the light emitting element(s) 2910 may emit red-colored light to indicate that the duty cycle should be changed, or a green-colored light to indicate that the duty cycle is correct. In some examples, the light emitting element(s) 2910 may flash to indicate that the duty cycle is incorrect, and may emit a sustained light, or no light, to indicate that the duty cycle is correct. These are merely examples and the light emitting element(s) 2910 may be configured to provide visual feedback relating to the duty cycle in other ways. In some examples, the examples of a visual signal(s) 1512, an audible signal(s) 1514, and/or a haptic signal(s) 1516 may be output as guidance for the rescuer 204 based at least in part on sound signals obtained via the auscultation device 2902.

In some examples, sounds associated with the heart of the subject 104 may be recorded (e.g., using the auscultation device(s) 2802 or the auscultation device(s) 2902) to generate audio signal(s) indicative of heart sounds, and the audio signal(s) can be analyzed to determine whether to dynamically adjust the duty cycle of chest compressions or not. In some examples, the audio signal(s) indicative of heart sounds can be analyzed to detect closure of the aortic valve, and, if aortic valve closure is detected before an end of the compression phase, the processor(s) of the CPR system may determine to adjust the duty cycle (e.g., by decreasing the compression phase, and/or by changing the duty cycle to a duty cycle with a compression phase that is shorter than the decompression phase).

FIG. 30 illustrates audio signals generated during administration of CPR to a subject 104 by a mechanical chest compression device 102. For example, the subject 104 may be in cardiac arrest (e.g., in VF) and the mechanical chest compression device 102 may be secured around the subject 104 and may be administering CPR to the subject 104 when the audio signals in FIG. 30 are generated. A first audio signal 3000 depicted in FIG. 30 is generated using an auscultation device(s) 2802 disposed on a thorax of the subject 104. For example, the first audio signal 3000 can be generated using the auscultation device 2802(1) disposed on the chest of the subject 104 near the heart, and/or using the auscultation device 2802(2) disposed on the back of the subject 104 near the heart. Accordingly, at least a portion(s) of the first audio signal 3000 may be indicative of heart sounds of the subject 104 that occur during ongoing CPR. It is to be appreciated that the first audio signal 3000 is merely an example, and that, depending on the characteristics of the auscultation device(s) 2802 (e.g., microphone, digital stethoscope, etc.), and/or depending on the frequency range selected in the audio recording, and/or depending on the frequency spectrum analyzed, more detailed (or less detailed) heart sound information may be exhibited in the first audio signal 3000.

A second audio signal 3002 depicted in FIG. 30 is generated at the same time as the first audio signal 3000, and is generated using a microphone(s) in an environment of the subject 104. For example, the second audio signal 3002 can be generated using the microphone 2808(1) disposed on the main portion 2810 of the mechanical chest compression device 102. Accordingly, the second audio signal 3002 may be indicative of sounds in the vicinity of the subject 104 (e.g., the sound(s) made by the operation of the mechanical chest compression device 102, such as the sound of the motor 1404 during movement of the chest compressing mechanism 106 (e.g., piston 1405)).

The audio signals 3000, 3002 (e.g., recordings) are shown as spanning the length of two sequential compression-decompression cycles 110, which include a first compression-decompression cycle 110(A) followed by a second, subsequent compression-decompression cycle 110(B). The first sound sequence exhibited in the second audio signal 3002 represents the sound of the mechanical chest compression device 102 when the chest compressing mechanism 106 (e.g., piston 1405) is moving downwards (e.g. during the initial portion of the compression phase 112). When this first sound sequence has ended, the chest compressing mechanism 106 (e.g., piston 1405) of the mechanical chest compression device 102 has reached its target/end position during the compression phase 112 (e.g. the chest has been compressed 53 mm from the start position) indicating the start of a first hold period 302(1)(A) in which the chest compressing mechanism 106 (e.g., piston 1405) and the chest are at the end position where they are being held down until the start of the decompression phase 114.

After this first hold period 302(1)(A), a second sound sequence is exhibited in the second audio signal 3002. This second sound sequence represents the sound of the mechanical chest compression device 102 when the chest compressing mechanism 106 (e.g., piston 1405) is moving upwards (e.g. during the initial portion of the decompression phase 114). When this second sound sequence has ended, the chest compressing mechanism 106 (e.g., piston 1405) of the mechanical chest compression device 102 has reached its top position (e.g., the starting position at 0 mm in the waveform 108 shown in FIG. 1) during the decompression phase 114 indicating the start of a second hold period 302(2)(A) in which the chest compressing mechanism 106 (e.g., piston 1405) and the chest are at the start position where they are being held up until the start of the next compression phase 112 of the second, subsequent compression-decompression cycle 110(B). The latter part of the second audio signal 3002 during the second compression-decompression cycle 110(B) includes a third sound sequence and a fourth sound sequence. These third and fourth sound sequences are similar to the first and second sound sequences exhibited during the first compression-decompression cycle 110(A). For example, a third sound sequence representing downward movement of the chest compressing mechanism 106 (e.g., piston 1405) of the mechanical chest compression device 102 precedes a first hold period 302(1)(B) at the end of the compression phase 112 of the second compression-decompression cycle 110(B), and a fourth sound sequence representing upward movement of the chest compressing mechanism 106 (e.g., piston 1405) of the mechanical chest compression device 102 precedes a second hold period 302(2)(B) at the end of the decompression phase 114 of the second compression-decompression cycle 110(B).

The four sound sequences, which represent the sounds made by the mechanical chest compression device 102 over the two compression-decompression cycles 110(A) and 110(B), are also exhibited in the first audio signal 3000. The four sound sequences occur in the first audio signal 3000 at the same respective times as they occur in the second audio signal 3002. In addition to these four device-created sound sequences, the first audio signal 3000 also exhibits internal body sounds associated with the heart of the subject 104, and, more specifically, the first audio signal 3000 exhibits the sound of flowing blood as the blood is ejected out from the heart during the compression phase 112 of each compression-decompression cycle 110. For example, during the first hold period 302(1)(A) of the compression phase 112 of the first compression-decompression cycle 110(A) (e.g., after the chest compressing mechanism 106 (e.g., piston 1405) of the mechanical chest compression device 102 has reached its target/end position during the compression phase 112), a heart sound signal is exhibited in the first audio signal 3000. This heart sound signal is exhibited in the first audio signal 3000 until the start of the decompression phase 114 of the first compression-decompression cycle 110(A) (e.g., this heart sound signal is present between the two time points S1 and S2 in FIG. 8 during which blood is being ejected from the heart). Thus, this heart sound signal is the sound created by the flow of blood (e.g., turbulent flow of blood) out from the heart when the blood is ejected from the ventricle(s) caused by the compression of the ventricle(s) during the compression phase 112 of the first compression-decompression cycle 110(A). Accordingly, the heart sound signal exhibited in the first audio signal 3000 during the first hold period 302(1)(A) may represent a blood flow parameter.

Now looking at the second half of the first audio signal 3000, during the second compression-decompression cycle 110(B), a similar heart sound signal is exhibited in the first audio signal 3000 during the first hold period 302(1)(B) of the compression phase 112 of the second compression-decompression cycle 110(B). In contrast with the similar heart sound signal exhibited during the first compression-decompression cycle 110(A), the heart sound signal exhibited during the second compression-decompression cycle 110(B) disappears prior to the start of the decompression phase 114 of the second compression-decompression cycle 110(B), as indicated by the highlighted area in FIG. 30 indicating the absence of blood flow sound 3004. In other words, the absence of blood flow sound 3004 during latter part of the first hold period 302(1)(B) of the compression phase 112 of the second compression-decompression cycle 110(B) may indicate that blood flow out of the heart has stopped for a period of time at the end of the compression phase 112 prior to the beginning of the decompression phase 114. Ejection of blood from the heart likely stopped towards the end of the first hold period 302(1)(B) due to the closing of the aortic valve at the time point of the beginning of the highlighted area indicating the absence of blood flow sound 3004. Thus, the heart sound signal exhibited in the first audio signal 3000 during the first hold period 302(1)(B) may represent a blood flow parameter, which can be used to detect the time point of the closing of the aortic valve of the heart of the subject 104 who is receiving CPR treatment via the mechanical chest compression device 102 due to the absence of blood flow sound 3004.

One reason why the aortic valve closure is detected in the first audio signal 3000 during the second compression-decompression cycle 110(B) and is not detected during the first compression-decompression cycle 110(A) is because two different duty cycles are implemented by the mechanical chest compression device 102 during the respective compression-decompression cycles 110(A) and 110(B). For example, the first compression-decompression cycle 110(A) may be associated with a 40:60 duty cycle (e.g., 40% of the time in compression phase 112 and 60% of the time in decompression phase 114), and the second compression-decompression cycle 110(B) may be associated with a 50:50 duty cycle (e.g., 50% of the time in compression phase 112 and 50% of the time in decompression phase 114). As mentioned above, a 50:50 duty cycle may be a suboptimal implementation, in some cases, since the aortic valve is likely to close before the end of the compression phase 112. This is illustrated by the absence of blood flow sound 3004 in the first audio signal 3000 during the latter part of the first hold period 302(1)(B) of the compression phase 112 of the second compression-decompression cycle 110(B). A 40:60 duty cycle, on the other hand, can be a better choice for the duty cycle of chest compressions, as evidenced by the timing of the closure of the aortic valve, which coincides with the start of the decompression phase 114. This is illustrated by the presence of blood flow sound in the first audio signal 3000 during the entirety of the first hold period 302(1)(A) of the compression phase 112 of the first compression-decompression cycle 110(B). Accordingly, blood flow through the aortic valve can be detected in a heart sound signal (e.g., the heart sound signals exhibited in the first audio signal 3000 during the first hold periods 302(1)(A) and 302(1)(B)). Furthermore, when such a heart sound signal disappears or is otherwise absent during the compression phase 112 (e.g., the highlighted area indicating absence of blood flow sound 3004), the processor(s) 1414 of the mechanical chest compression device 102 may detect closure of the aortic valve.

More generally, the example of FIG. 30 demonstrates that turbulent blood flow creates a detectable heart sound that may be recorded using an auscultation device(s), such as the auscultation device(S) 2802 or the auscultation device 2902. The detection of the presence or absence of this blood flow sound signal may be used in order to detect if the aortic valve is open or closed. The heart sound signal may be analyzed during the compression phase 112 in order to detect the presence of blood flow through the aortic valve. The time point of the aortic valve closure may be determined by the time point when sound from blood flow disappears, since, when the aortic valve closes, no blood will flow through the aortic valve anymore. When the mechanical chest compression device 102 is being used to administer CPR to the subject 104, this heart sound signal can be used to determine the optimal duty cycle and to automatically control and adjust the duty cycle of the mechanical chest compression device 102 during ongoing CPR (e.g., by performing the process 900 of FIG. 9 and/or the process 1000 of FIG. 10). When a CPR duty cycle guidance system (e.g., the system 202) is being used to provide guidance to a rescuer 204 who is manually administering CPR to the subject 104, this heart sound signal can be used to determine the optimal duty cycle and to output a signal (e.g., the visual signal(s) 1512, audible signal(s) 1514, and/or haptic signal(s) 1516) as guidance for the rescuer 204 to adjust the duty cycle, as described in detail above (e.g., by performing the process 1000 of FIG. 10, the process 1100 of FIG. 11, etc.).

In some examples, the heart sound signal(s) (e.g., blood flow sounds) illustrated in FIG. 30 can be used to determine the quality of CPR being performed (e.g., whether high quality CPR is being performed or not). For example, the heart sound signal exhibited in the first audio signal 3000 not only shows when blood flow is present and created, but also how much blood flow is being created since the energy in the heart sound signal provides an estimate of the blood flow created (e.g., the stroke volume and the cardiac output). An estimation of the created blood flow is also an estimation of the quality of CPR. The higher the created blood flow (e.g., energy in the heart sound signal), the better the performance of CPR. The quality of CPR may be obtained from different measures related to the energy of the heart sound signal in the first audio signal 3000 including, without limitation, the maximum and/or mean and/or median of the amplitude, magnitude, root-mean-square (RMS), standard deviation, variance, or entropy. These different measures may be obtained in a defined time segment of the heart sound signal exhibited in the first audio signal 3000 and/or during the entire segment with “detected blood flow” (e.g., during the compression phase 112 until the time point of aortic valve closure).

Although there are advantages of using multiple audio capture devices (e.g., using the auscultation device(s) 2802 in conjunction with the microphone 2808(1), or using the auscultation device 2902 in conjunction with the microphone 2906), such as identifying and filtering out the sounds of the mechanical chest compression device 102 (e.g., chest compression artifacts) from the first audio signal 3000, improving the SNR, and/or the like, it is to be appreciated that a single auscultation device can be used for determining the optimal/preferred duty cycle and for dynamically adjusting the duty cycle and/or outputting feedback or guidance to a rescuer to adjust the duty cycle. For example, the mechanical chest compression device 102 may include and/or utilize the auscultation device 2802(1) by itself to generate the first audio signal 3000 depicted in FIG. 30, and the processor(s) 1414 may analyze the first audio signal 3000 to determine if there is an absence of blood flow sound during the compression phase 112 of a compression-decompression cycle 110, and, if so, adjust the duty cycle (e.g., by decreasing the compression phase 112). As another example, the CPR feedback device 206 may include and/or utilize the auscultation device 2902 by itself to generate an audio signal similar to the first audio signal 3000 depicted in FIG. 30, and the processor(s) of the CPR feedback system 202 may analyze the audio signal to determine if there is an absence of blood flow sound during the compression phase 112 of a compression-decompression cycle 110, and, if so, output a signal(s) as guidance for the rescuer 204 to adjust the duty cycle (e.g., by decreasing the compression phase 112). In some examples, if blood flow sound is present during the entirety of the compression phase 112 of a compression-decompression cycle 110, a CPR duty cycle guidance system may output a signal(s) as feedback or guidance for the rescuer 204 (e.g., indicating that they are adhering to the target duty cycle of chest compressions). In some examples, an iterative optimization algorithm (e.g., the process 1000 of FIG. 10) is utilized to determine, based on the presence of blood flow sound during the entirety of the compression phase 112, whether the compression phase 112 should be increased. That is, the presence of blood flow sound throughout the entirety of the compression phase 112 may not be enough information, by itself, to determine whether the duty cycle is optimal or whether the duty cycle should be adjusted, and the system may adjust or output a signal(s) for the rescuer to adjust the duty cycle by increasing the compression phase 112 until absence of blood flow sound is detected in the audio signal (e.g., the first audio signal 3000) during the compression phase 112, and then converge on the optimal duty cycle after this absence of blood flow sound is detected for a duty cycle with a compression phase that is slightly too long.

### Example Clauses

1. A defibrillator comprising: an output device; and a processor configured to: generate a signal indicative of a duty cycle of chest compressions, wherein the duty cycle represents a ratio of a first time period of a compression phase of a compression-decompression cycle to a second time period of a decompression phase of the compression-decompression cycle; and cause the output device to output the signal as guidance for a rescuer who is manually administering the chest compressions to a subject.
2. The defibrillator of clause 1, wherein: the duty cycle is a target duty cycle;
the output device comprises a speaker; the signal comprises an audible signal; and the audible signal is output via the speaker as a sustained sound that changes pitch in accordance with a waveform exhibiting the target duty cycle.
3. The defibrillator of clause 1, wherein: the duty cycle is a target duty cycle; the output device comprises a display; the signal comprises a visual signal; and the visual signal is indicative of the target duty cycle and is output as a moving icon that tracks along a waveform exhibiting the target duty cycle.
4. The defibrillator of clause 1, further comprising electrode pads configured to be attached to a thorax of the subject, wherein the duty cycle is a target duty cycle, the output device comprises a display, the signal comprises a visual signal, and the processor is further configured to: determine an administered duty cycle by analyzing an impedance signal generated using the electrode pads; and determine, by comparing the administered duty cycle to the target duty cycle, to provide guidance to the rescuer to adjust the administered duty cycle, wherein the visual signal is output via the display as guidance for the rescuer to adjust the administered duty cycle.
5. The defibrillator of any one of clauses 1 to 4, wherein the processor is communicatively coupled to a haptic actuator that is configured to be disposed on, or in proximity to, a chest of the subject, the duty cycle is a target duty cycle, and the processor is further configured to: generate a haptic signal indicative of the target duty cycle; and cause the haptic actuator to output the haptic signal as guidance for the rescuer.
6. A cardiopulmonary resuscitation (CPR) duty cycle guidance system comprising: an output device; and a processor configured to: generate a signal indicative of a duty cycle of chest compressions, wherein the duty cycle represents a ratio of a first time period of a compression phase of a compression-decompression cycle to a second time period of a decompression phase of the compression-decompression cycle; and cause the output device to output the signal as guidance for a rescuer who is manually administering the chest compressions to a subject.
7. The CPR duty cycle guidance system of clause 6, wherein: the duty cycle is a target duty cycle;
the output device comprises a display; the signal comprises a visual signal; and causing the output device to output the signal comprises causing the display to present the visual signal as a moving icon that tracks along a waveform exhibiting the target duty cycle.
8. The CPR duty cycle guidance system of clause 7, wherein: the waveform spans multiple compression-decompression cycles; and the moving icon continues moving in a rightward direction across the display as the moving icon transitions from a first compression-decompression cycle to a second compression-decompression cycle.
9. The CPR duty cycle guidance system of clause 7 or 8, wherein: the waveform is a first waveform; and the processor is further configured to: determine an administered duty cycle of the chest compressions that the rescuer manually administered to the subject; and cause the display to present a second waveform exhibiting the administered duty cycle, wherein the second waveform is overlaid on top of the first waveform and aligned with the first waveform on a time axis.
10. The CPR duty cycle guidance system of clause 7 or 8, wherein the processor is further configured to: determine an administered duty cycle of the chest compressions that the rescuer manually administered to the subject; determine, by comparing the administered duty cycle to the target duty cycle, to provide guidance to the rescuer to adjust the administered duty cycle; and
cause the display to highlight at least a portion of the waveform in a color as guidance for the rescuer to adjust the administered duty cycle.
11. The CPR duty cycle guidance system of clause 6, wherein: the duty cycle is an administered duty cycle of the chest compressions that the rescuer manually administered to the subject; the output device comprises a display; the signal comprises an impedance signal generated using electrode pads configured to be attached to a thorax of the subject; and causing the output device to output the signal comprises causing the display to present the impedance signal.
12. The CPR duty cycle guidance system of clause 6, wherein: the duty cycle is a target duty cycle; the output device comprises a speaker; the signal comprises an audible signal; and causing the output device to output the signal comprises causing the speaker to output the audible signal as: a sustained sound that changes pitch or volume in accordance with a waveform exhibiting the target duty cycle; a first sound at a first predetermined time prior to a start of the compression phase followed by a second sound at a second predetermined time prior to a start of the decompression phase; or the first sound at a first dynamically determined time prior to the start of the compression phase followed by the second sound at a second dynamically determined time prior to the start of the decompression phase.
13. The CPR duty cycle guidance system of clause 6, wherein: the duty cycle is a target duty cycle; the output device comprises a haptic actuator; the signal comprises a haptic signal; and

causing the output device to output the signal comprises causing the haptic actuator to output the haptic signal as: a sustained vibration that changes frequency or amplitude in accordance with a waveform exhibiting the target duty cycle; a first vibration at a first predetermined time prior to a start of the compression phase followed by a second vibration at a second predetermined time prior to a start of the decompression phase; or the first vibration at a first dynamically determined time prior to the start of the compression phase followed by the second vibration at a second dynamically determined time prior to the start of the decompression phase.
14. The CPR duty cycle guidance system of clause 6, wherein: the duty cycle is a target duty cycle; the signal is a first signal; and the processor is further configured to: determine an administered duty cycle of the chest compressions that the rescuer manually administered to the subject; determine, by comparing the administered duty cycle to the target duty cycle, to provide guidance to the rescuer to adjust the administered duty cycle; and cause the output device to output a second signal as guidance for the rescuer to adjust the duty cycle.
15. The CPR duty cycle guidance system of clause 14, wherein determining the administered duty cycle comprises computing an average duty cycle of the chest compressions that the rescuer manually administered to the subject over N compression-decompression cycles, wherein N is an integer greater than one.
16. The CPR duty cycle guidance system of clause 14 or 15, wherein determining to provide guidance to the rescuer to adjust the administered duty cycle is based on a determination, from comparing the administered duty cycle to the target duty cycle, that the rescuer is not keeping a chest of the subject in a compressed state or in a decompressed state for a threshold period of time.
17. The CPR duty cycle guidance system of any one of clauses 6 to 16, wherein the first time period is shorter than the second time period.
18. A method comprising: generating, by a processor of a cardiopulmonary resuscitation (CPR) duty cycle guidance system, a signal indicative of a duty cycle of chest compressions, wherein the duty cycle represents a ratio of a first time period of a compression phase of a compression-decompression cycle to a second time period of a decompression phase of the compression-decompression cycle; and causing, by the processor, an output device to output the signal as guidance for a rescuer who is manually administering the chest compressions to a subject.
19. The method of clause 18, wherein: the duty cycle is a target duty cycle; the output device comprises a display; the signal comprises a visual signal; and causing the output device to output the signal comprises causing the display to present the visual signal as a moving icon that tracks along a waveform exhibiting the target duty cycle.
20. The method of clause 19, wherein: the waveform is a first waveform; and the method further comprises: determining, by the processor, an administered duty cycle of the chest compressions that the rescuer manually administered to the subject; and causing, by the processor, the display to present a second waveform exhibiting the administered duty cycle, wherein the second waveform is overlaid on top of the first waveform and aligned with the first waveform on a time axis.
21. The method of clause 18, wherein: the duty cycle is a target duty cycle; the output device comprises a haptic actuator; the signal comprises a haptic signal; and causing the output device to output the signal comprises causing the haptic actuator to output the haptic signal as: a sustained vibration that changes frequency or amplitude in accordance with a waveform exhibiting the target duty cycle; a first vibration at a first predetermined time prior to a start of the compression phase followed by a second vibration at a second predetermined time prior to a start of the decompression phase; or the first vibration at a first dynamically determined time prior to the start of the compression phase followed by the second vibration at a second dynamically determined time prior to the start of the decompression phase.
22. The method of clause 18, wherein: the duty cycle is a target duty cycle;
the signal is a first signal; and the method further comprises: determining, by the processor, an administered duty cycle of the chest compressions that the rescuer manually administered to the subject; determining, by the processor, and by comparing the administered duty cycle to the target duty cycle, to provide guidance to the rescuer to adjust the administered duty cycle; and causing, by the processor, the output device to output a second signal as guidance for the rescuer to adjust the duty cycle.

While the example clauses described above are described with respect to one particular implementation, it should be understood that, in the context of this document, the content of the example clauses can also be implemented via a method, device, system, computer-readable medium, and/or another implementation. Additionally, any one of examples 1-22 may be implemented alone or in combination with any other of the examples 1-22.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, the term “based on” is equivalent to “based at least partly on,” unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain implementations are described herein, including the best mode known to the inventors for carrying out implementations of the disclosure. Of course, variations on these described implementations will become apparent to those of ordinary skill in the art upon reading the foregoing description. The inventors expect skilled artisans to employ such variations as appropriate, and the inventors intend for implementations to be practiced otherwise than specifically described herein. Accordingly, the scope of this disclosure includes all modifications and equivalents of the subject matter recited in the claims appended hereto as permitted by applicable law. Moreover, any combination of the above-described elements in all possible variations thereof is encompassed by implementations of the disclosure unless otherwise indicated herein or otherwise clearly contradicted by context.

What is claimed is:

1. A defibrillator comprising:
an output device; and
a processor configured to:
generate a signal indicative of a duty cycle of chest compressions, wherein the duty cycle represents a ratio of a first time period of a compression phase of a compression-decompression cycle to a second time period of a decompression phase of the compression-decompression cycle; and
cause the output device to output the signal as guidance for a rescuer who is manually administering the chest compressions to a subject.

2. The defibrillator of claim 1, wherein:
the duty cycle is a target duty cycle;
the output device comprises a speaker;
the signal comprises an audible signal; and
the audible signal is output via the speaker as a sustained sound that changes pitch in accordance with a waveform exhibiting the target duty cycle.

3. The defibrillator of claim 1, wherein:
the duty cycle is a target duty cycle;
the output device comprises a display;
the signal comprises a visual signal; and
the visual signal is indicative of the target duty cycle and is output as a moving icon that tracks along a waveform exhibiting the target duty cycle.

4. The defibrillator of claim 1, further comprising electrode pads configured to be attached to a thorax of the subject, wherein the duty cycle is a target duty cycle, the output device comprises a display, the signal comprises a visual signal, and the processor is further configured to:
determine an administered duty cycle by analyzing an impedance signal generated using the electrode pads; and
determine, by comparing the administered duty cycle to the target duty cycle, to provide guidance to the rescuer to adjust the administered duty cycle,
wherein the visual signal is output via the display as guidance for the rescuer to adjust the administered duty cycle.

5. The defibrillator of claim 1, wherein the processor is communicatively coupled to a haptic actuator that is configured to be disposed on, or in proximity to, a chest of the subject, the duty cycle is a target duty cycle, and the processor is further configured to:
generate a haptic signal indicative of the target duty cycle; and
cause the haptic actuator to output the haptic signal as guidance for the rescuer.

6. A cardiopulmonary resuscitation (CPR) duty cycle guidance system comprising:
an output device; and
a processor configured to:
generate a signal indicative of a duty cycle of chest compressions, wherein the duty cycle represents a ratio of a first time period of a compression phase of a compression-decompression cycle to a second time period of a decompression phase of the compression-decompression cycle; and
cause the output device to output the signal as guidance for a rescuer who is manually administering the chest compressions to a subject.

7. The CPR duty cycle guidance system of claim 6, wherein:
the duty cycle is a target duty cycle;
the output device comprises a display;
the signal comprises a visual signal; and
causing the output device to output the signal comprises causing the display to present the visual signal as a moving icon that tracks along a waveform exhibiting the target duty cycle.

8. The CPR duty cycle guidance system of claim 7, wherein:
the waveform spans multiple compression-decompression cycles; and
the moving icon continues moving in a rightward direction across the display as the moving icon transitions from a first compression-decompression cycle to a second compression-decompression cycle.

9. The CPR duty cycle guidance system of claim 7, wherein:
the waveform is a first waveform; and
the processor is further configured to:
determine an administered duty cycle of the chest compressions that the rescuer manually administered to the subject; and
cause the display to present a second waveform exhibiting the administered duty cycle, wherein the second waveform is overlaid on top of the first waveform and aligned with the first waveform on a time axis.

10. The CPR duty cycle guidance system of claim 7, wherein the processor is further configured to:
determine an administered duty cycle of the chest compressions that the rescuer manually administered to the subject;
determine, by comparing the administered duty cycle to the target duty cycle, to provide guidance to the rescuer to adjust the administered duty cycle; and
cause the display to highlight at least a portion of the waveform in a color as guidance for the rescuer to adjust the administered duty cycle.

11. The CPR duty cycle guidance system of claim 6, wherein:
the duty cycle is an administered duty cycle of the chest compressions that the rescuer manually administered to the subject;
the output device comprises a display;
the signal comprises an impedance signal generated using electrode pads configured to be attached to a thorax of the subject; and
causing the output device to output the signal comprises causing the display to present the impedance signal.

12. The CPR duty cycle guidance system of claim 6, wherein:
the duty cycle is a target duty cycle;
the output device comprises a speaker;
the signal comprises an audible signal; and
causing the output device to output the signal comprises causing the speaker to output the audible signal as:
a sustained sound that changes pitch or volume in accordance with a waveform exhibiting the target duty cycle;
a first sound at a first predetermined time prior to a start of the compression phase followed by a second sound at a second predetermined time prior to a start of the decompression phase; or
the first sound at a first dynamically determined time prior to the start of the compression phase followed by the second sound at a second dynamically determined time prior to the start of the decompression phase.

13. The CPR duty cycle guidance system of claim 6, wherein:
the duty cycle is a target duty cycle;
the output device comprises a haptic actuator;
the signal comprises a haptic signal; and
causing the output device to output the signal comprises causing the haptic actuator to output the haptic signal as:
a sustained vibration that changes frequency or amplitude in accordance with a waveform exhibiting the target duty cycle;
a first vibration at a first predetermined time prior to a start of the compression phase followed by a second vibration at a second predetermined time prior to a start of the decompression phase; or
the first vibration at a first dynamically determined time prior to the start of the compression phase followed by the second vibration at a second dynamically determined time prior to the start of the decompression phase.

14. The CPR duty cycle guidance system of claim 6, wherein:
the duty cycle is a target duty cycle;
the signal is a first signal; and
the processor is further configured to:
determine an administered duty cycle of the chest compressions that the rescuer manually administered to the subject;
determine, by comparing the administered duty cycle to the target duty cycle, to provide guidance to the rescuer to adjust the administered duty cycle; and
cause the output device to output a second signal as guidance for the rescuer to adjust the duty cycle.

15. The CPR duty cycle guidance system of claim 14, wherein determining the administered duty cycle comprises computing an average duty cycle of the chest compressions that the rescuer manually administered to the subject over N compression-decompression cycles, wherein N is an integer greater than one.

16. The CPR duty cycle guidance system of claim 14, wherein determining to provide guidance to the rescuer to adjust the administered duty cycle is based on a determination, from comparing the administered duty cycle to the target duty cycle, that the rescuer is not keeping a chest of the subject in a compressed state or in a decompressed state for a threshold period of time.

17. The CPR duty cycle guidance system of claim 6, wherein the first time period is shorter than the second time period.

18. A method comprising:
generating, by a processor of a cardiopulmonary resuscitation (CPR) duty cycle guidance system, a signal indicative of a duty cycle of chest compressions, wherein the duty cycle represents a ratio of a first time period of a compression phase of a compression-decompression cycle to a second time period of a decompression phase of the compression-decompression cycle; and
causing, by the processor, an output device to output the signal as guidance for a rescuer who is manually administering the chest compressions to a subject.

19. The method of claim 18, wherein:
the duty cycle is a target duty cycle;
the output device comprises a display;
the signal comprises a visual signal; and
causing the output device to output the signal comprises causing the display to present the visual signal as a moving icon that tracks along a waveform exhibiting the target duty cycle.

20. The method of claim 19, wherein:
the waveform is a first waveform; and
the method further comprises:
determining, by the processor, an administered duty cycle of the chest compressions that the rescuer manually administered to the subject; and
causing, by the processor, the display to present a second waveform exhibiting the administered duty cycle, wherein the second waveform is overlaid on top of the first waveform and aligned with the first waveform on a time axis.

21. The method of claim 18, wherein:
the duty cycle is a target duty cycle;
the output device comprises a haptic actuator;
the signal comprises a haptic signal; and
causing the output device to output the signal comprises causing the haptic actuator to output the haptic signal as:
a sustained vibration that changes frequency or amplitude in accordance with a waveform exhibiting the target duty cycle;
a first vibration at a first predetermined time prior to a start of the compression phase followed by a second vibration at a second predetermined time prior to a start of the decompression phase; or
the first vibration at a first dynamically determined time prior to the start of the compression phase followed by the second vibration at a second dynamically determined time prior to the start of the decompression phase.

22. The method of claim 18, wherein:
the duty cycle is a target duty cycle;
the signal is a first signal; and
the method further comprises:
determining, by the processor, an administered duty cycle of the chest compressions that the rescuer manually administered to the subject;
determining, by the processor, and by comparing the administered duty cycle to the target duty cycle, to provide guidance to the rescuer to adjust the administered duty cycle; and
causing, by the processor, the output device to output a second signal as guidance for the rescuer to adjust the duty cycle.
